SEQUENCE LISTING (80 sequences)

SEQ ID NO: 1 - protein, 19 aa, synthetic construct
MAWVWTLLFLMAAAQSAQA

SEQ ID NO: 2 - DNA, 57 bp, synthetic construct
  source 1..57: mol_type = other DNA
atggcttgggtgtggaccttgctattcctgatggcagctgcccaaagtgcccaagca

SEQ ID NO: 3 - protein, 121 aa, synthetic construct
QIQLVQSGPELKKPGETVKISCKASGYIFTNYGMNWVRQAPGKGLKWMGWINSYTGEATYTDDLKGRFAFSLESSASTAYLQINNLKNEDTATYFCAREGYGDYGYSFDYWGQGTTLTVSS

SEQ ID NO: 4 - protein, 5 aa, synthetic construct
NYGMN

SEQ ID NO: 5 - protein, 17 aa, synthetic construct
WINSYTGEATYTDDLKG

SEQ ID NO: 6 - protein, 12 aa, synthetic construct
EGYGDYGYSFDY

SEQ ID NO: 7 - protein, 8 aa, synthetic construct
GYIFTNYG

SEQ ID NO: 8 - protein, 8 aa, synthetic construct
INSYTGEA

SEQ ID NO: 9 - protein, 14 aa, synthetic construct
AREGYGDYGYSFDY

SEQ ID NO: 10 - protein, 7 aa, synthetic construct
GYIFTNY

SEQ ID NO: 11 - protein, 6 aa, synthetic construct
NSYTGE

SEQ ID NO: 12 - protein, 330 aa, synthetic construct
AKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 13 - DNA, 990 bp, synthetic construct
  source 1..990: mol_type = other DNA
gccaaaacaacagccccatcggtctatccactggcccctgtgtgtggagatacaactggctcctcggtgactctaggatgcctggtcaagggttatttccctgagccagtgaccttgacctggaactctggatccctgtccagtggtgtgcacaccttcccagctgtcctgcagtctgacctctacaccctcagcagctcagtgactgtaacctcgagcacctggcccagccagtccatcacctgcaatgtggcccacccggcaagcagcaccaaggtggacaagaaaattgagcccagagggcccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaa

SEQ ID NO: 14 - protein, 107 aa, synthetic construct
DIQMTQSPASLSASVGETVTITCRATKNIYTYLAWYQQKQGKSPQFLVYNAKTLAEGVPSRFSGSGSGTQFSLKINSLQPEDFGNYYCQHHYGTPRTFGGGTKLEIK

SEQ ID NO: 15 - DNA, 321 bp, synthetic construct
  source 1..321: mol_type = other DNA
gacatccagatgactcagtctccagcctccctatctgcatctgtgggggaaactgtcaccatcacatgtcgagcaactaagaatatttacacttatttagcatggtatcagcagaaacagggaaaatctcctcagttcctggtctataatgcaaaaaccttagcagaaggtgtgccatcaaggttcagtggcagtggatcaggcacacagttttctctgaagatcaatagcctgcagcctgaagattttgggaattattactgtcaacatcattatggaactcctcggacgttcggtggaggcaccaagctggaaatcaaa

SEQ ID NO: 16 - protein, 11 aa, synthetic construct
RATKNIYTYLA

SEQ ID NO: 17 - protein, 7 aa, synthetic construct
NAKTLAE

SEQ ID NO: 18 - protein, 9 aa, synthetic construct
QHHYGTPRT

SEQ ID NO: 19 - protein, 6 aa, synthetic construct
KNIYTY

SEQ ID NO: 20 - protein, 107 aa, synthetic construct
RADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 21 - DNA, 321 bp, synthetic construct
  source 1..321: mol_type = other DNA
cgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgt

SEQ ID NO: 22 - protein, 19 aa, synthetic construct
MNFGLSLIFLVLILKGVQC

SEQ ID NO: 23 - DNA, 57 bp, synthetic construct
  source 1..57: mol_type = other DNA
atgaacttcgggctcagcttgattttccttgtcctcattttaaaaggtgtccagtgt

SEQ ID NO: 24 - protein, 118 aa, synthetic construct
EVQLVESGGGLVKPGGSLKLSCAASGFTFSTYAMSWVRQSPEKRLEWVAEISNGGLYTYYPDTVTGRFTISRDNVKNILYLEMSSLRSEDTAIYYCIRENRNWYFDLWGAGTTVTVSS

SEQ ID NO: 25 - protein, 5 aa, synthetic construct
TYAMS

SEQ ID NO: 26 - protein, 17 aa, synthetic construct
EISNGGLYTYYPDTVTG

SEQ ID NO: 27 - protein, 9 aa, synthetic construct
ENRNWYFDL

SEQ ID NO: 28 - protein, 8 aa, synthetic construct
GFTFSTYA

SEQ ID NO: 29 - protein, 8 aa, synthetic construct
ISNGGLYT

SEQ ID NO: 30 - protein, 11 aa, synthetic construct
IRENRNWYFDL

SEQ ID NO: 31 - protein, 7 aa, synthetic construct
GFTFSTY

SEQ ID NO: 32 - protein, 6 aa, synthetic construct
SNGGLY

SEQ ID NO: 33 - DNA, 354 bp, synthetic construct
  source 1..354: mol_type = other DNA
gaagtgcagctggtggagtctgggggaggcttagtgaagcctggagggtccctgaaactctcctgtgcagcctctggattcaccttcagtacctatgccatgtcttgggttcgccagtctccagagaagaggctggagtgggtcgcagaaattagtaatggtggtctttacacctactatccagacactgtgacgggccgattcaccatctccagagacaatgtcaagaatatcttatacctggaaatgagcagtctgaggtctgaggacacggccatatattactgtataagagaaaataggaactggtacttcgatctctggggcgcagggaccacggtcaccgtctcctca

SEQ ID NO: 34 - protein, 324 aa, synthetic construct
AKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 35 - DNA, 972 bp, synthetic construct
  source 1..972: mol_type = other DNA
gccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaa

SEQ ID NO: 36 - protein, 112 aa, synthetic construct
DVLMTQTPLSLPVSLGDQASISCRSSQSIVNSNGNTYLEWYLQKPGQSPKLLIYKVSNRFSGVPDRFSGSGSGTDFTLKISRVEAEDLGVYYCFQGSHVPLTFGAGTKLELK

SEQ ID NO: 37 - protein, 16 aa, synthetic construct
RSSQSIVNSNGNTYLE

SEQ ID NO: 38 - protein, 7 aa, synthetic construct
KVSNRFS

SEQ ID NO: 39 - protein, 9 aa, synthetic construct
FQGSHVPLT

SEQ ID NO: 40 - protein, 11 aa, synthetic construct
QSIVNSNGNTY

SEQ ID NO: 41 - protein, 119 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKVAPYFAPFDYWGQGTLVTVSS

SEQ ID NO: 42 - protein, 109 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGNPPLYTFGQGTKVEIK

SEQ ID NO: 43 - protein, 5 aa, synthetic construct
SYAMS

SEQ ID NO: 44 - protein, 17 aa, synthetic construct
AISGSGGSTYYADSVKG

SEQ ID NO: 45 - protein, 10 aa, synthetic construct
VAPYFAPFDY

SEQ ID NO: 46 - protein, 12 aa, synthetic construct
RASQSVSSSYLA

SEQ ID NO: 47 - protein, 7 aa, synthetic construct
GASSRAT

SEQ ID NO: 48 - protein, 10 aa, synthetic construct
QQYGNPPLYT

SEQ ID NO: 49 - protein, 348 aa, Homo sapiens
MSDLGAVISLLLWGRQLFALYSGNDVTDISDDRFPKPPEIANGYVEHLFRYQCKNYYRLRTEGDGVYTLNDKKQWINKAVGDKLPECEAVCGKPKNPANPVQRILGGHLDAKGSFPWQAKMVSHHNLTTGATLINEQWLLTTAKNLFLNHSENATAKDIAPTLTLYVGKKQLVEIEKVVLHPNYHQVDIGLIKLKQKVLVNERVMPICLPSKNYAEVGRVGYVSGWGQSDNFKLTDHLKYVMLPVADQYDCITHYEGSTCPKWKAPKSPVGVQPILNEHTFCVGMSKYQEDTCYGDAGSAFAVHDLEEDTWYAAGILSFDKSCAVAEYGVYVKVTSIQHWVQKTIAEN

SEQ ID NO: 50 - protein, 398 aa, Homo sapiens
MEGAALLRVSVLCIWMSALFLGVGVRAEEAGARVQQNVPSGTDTGDPQSKPLGDWAAGTMDPESSIFIEDAIKYFKEKVSTQNLLLLLTDNEAWNGFVAAAELPRNEADELRKALDNLARQMIMKDKNWHDKGQQYRNWFLKEFPRLKSELEDNIRRLRALADGVQKVHKGTTIANVVSGSLSISSGILTLVGMGLAPFTEGGSLVLLEPGMELGITAALTGITSSTMDYGKKWWTQAQAHDLVIKSLDKLKEVREFLGENISNFLSLAGNTYQLTRGIGKDIRALRRARANLQSVPHASASRPRVTEPISAESGEQVERVNEPSILEMSRGVKLTDVAPVSFFLVLDVVYLVYESKHLHEGAKSETAEELKKVAQELEEKLNILNNNYKILQADQEL

SEQ ID NO: 51 - protein, 243 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGNPPLYTFGQGTKVEIKGGGGSGGGGSGGGGSEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKVAPYFAPFDYWGQGTLVTVSS

SEQ ID NO: 52 - protein, 15 aa, synthetic construct
GGGGSGGGGSGGGGS

SEQ ID NO: 53 - protein, 11 aa, synthetic construct
YGRKKRRQRRR

SEQ ID NO: 54 - protein, 9 aa, synthetic construct
RKKRRQRRR

SEQ ID NO: 55 - protein, 16 aa, synthetic construct
RQIKIWFQNRRMKWKK

SEQ ID NO: 56 - protein, 119 aa, synthetic construct
QIQLVQSGPELKKPGETVKISCKASGFTFTDYSIHWVKQAPGKGLKWMGWKHTESGESTYADDFKGRFVFSLETSASTAYLQINNLKNEDTSTYFCARGANYGSLLDYWGQGTTLTVSS

SEQ ID NO: 57 - protein, 5 aa, synthetic construct
DYSIH

SEQ ID NO: 58 - protein, 17 aa, synthetic construct
WKHTESGESTYADDFKG

SEQ ID NO: 59 - protein, 10 aa, synthetic construct
GANYGSLLDY

SEQ ID NO: 60 - protein, 8 aa, synthetic construct
GFTFTDYS

SEQ ID NO: 61 - protein, 8 aa, synthetic construct
KHTESGES

SEQ ID NO: 62 - protein, 12 aa, synthetic construct
ARGANYGSLLDY

SEQ ID NO: 63 - protein, 7 aa, synthetic construct
GFTFTDY

SEQ ID NO: 64 - protein, 6 aa, synthetic construct
HTESGE

SEQ ID NO: 65 - protein, 112 aa, synthetic construct
DIVLTQSPASLAVSLGQRATISCRASKSVSTSGYSYMHWYQQKPGQSPKLLIYLASNLESGVPARFSGSGSGTDFTLNIHPVEEEDAATYYCQHNRELPLTFGAGTKLELKR

SEQ ID NO: 66 - protein, 15 aa, synthetic construct
RASKSVSTSGYSYMH

SEQ ID NO: 67 - protein, 7 aa, synthetic construct
LASNLES

SEQ ID NO: 68 - protein, 9 aa, synthetic construct
QHNRELPLT

SEQ ID NO: 69 - protein, 10 aa, synthetic construct
KSVSTSGYSY

SEQ ID NO: 70 - DNA, 336 bp, synthetic construct
  source 1..336: mol_type = other DNA
gacattgtgctgacacagtctcctgcttccttagctgtatctctggggcagagggccaccatctcatgcagggccagcaaaagtgtcagtacatctggctatagttatatgcactggtaccaacagaaaccaggacagtcacccaaactcctcatctatcttgcatccaacctagaatctggggtccctgccaggttcagtggcagtgggtctgggacagacttcaccctcaacatccatcctgtggaggaggaggatgctgcaacctattactgtcagcacaatagggagcttccgctcacgttcggtgctgggaccaagctggagctgaaacgg

SEQ ID NO: 71 - protein, 704 aa, synthetic construct
EVQLVESGGGLVKPGGSLKLSCAASGFTFSTYAMSWVRQSPEKRLEWVAEISNGGLYTYYPDTVTGRFTISRDNVKNILYLEMSSLRSEDTAIYYCIRENRNWYFDLWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSEIVLTQSPGTLSLSPGERATLSCRASQSVSSSYLAWYQQKPGQAPRLLIYGASSRATGIPDRFSGSGSGTDFTLTISRLEPEDFAVYYCQQYGNPPLYTFGQGTKVEIKGGGGSGGGGSGGGGSEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKVAPYFAPFDYWGQGTLVTVSS

SEQ ID NO: 72 - protein, 219 aa, synthetic construct
DVLMTQTPLSLPVSLGDQASISCRSSQSIVNSNGNTYLEWYLQKPGQSPKLLIYKVSNRFSGVPDRFSGSGSGTDFTLKISRVEAEDLGVYYCFQGSHVPLTFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 73 - DNA, 2112 bp, synthetic construct
  source 1..2112: mol_type = other DNA
gaagtgcaactggtggagtccgggggggggttagtgaaacccggcggatctctgaagttgagctgcgcagcttccggcttcaccttcagcacctacgccatgagctgggtcagacagagccctgagaagagactggagtgggtggccgagatcagcaacggagggctgtacacatactaccccgacaccgtgaccggaagatttaccatctctagagacaacgttaagaacatcctgtacctggagatgagcagcctgagaagcgaagacaccgccatctactactgcatcagagagaacagaaactggtacttcgacctgtggggggccggcaccacagtgactgtgagttccgcatccaccaagggtccctcagtattccccctggctcctagctccaagagcacgagcgggggcactgccgcactgggctgtctcgtcaaggactactttcctgagcctgtgacagtgagctggaacagcggggccctgacaagcggcgtccacaccttccccgccgtgctgcagagcagcggcctatattcacttagcagcgtggtgaccgtgcctagcagcagcctgggcacacagacctacatctgcaacgtgaaccacaagcctagcaacaccaaggtggacaagaaggtggagccgaagagctgtgacaagacgcatacgtgccctccctgccccgcacccgaactgttgggcgggccctcagtgttcctgttcccccccaagcccaaggacaccctgatgatcagcagaacccctgaggtgacctgcgtggtggtggacgtgagccacgaggaccctgaggtgaagttcaactggtacgtggacggcgtggaggtgcacaacgccaagaccaagcctagagaggagcagtacaacagcacctacagagtggtgagcgtgctgaccgtgctgcaccaagactggctgaacggcaaggagtacaagtgcaaggtgagcaacaaggccctgcccgcccccatcgagaagaccatcagcaaggccaaggggcagcctagagagccccaagtgtacaccctgccccctagcagagacgagctgaccaagaaccaagtgagcctgacctgtctggtgaagggtttctaccctagcgacatcgccgtggagtgggagagcaacgggcagcctgagaacaactacaagaccaccccccccgtgctggacagcgacggcagcttcttcctgtacagcaagctgaccgtggacaagagcagatggcagcaaggcaacgtgttcagctgcagcgtgatgcacgaggccctgcacaaccattacacgcagaagtcactgtcgctgtctcccggcggtggcggatctggggggggtggttctgggggtgggggttccgaaatcgtcctgacacaaagtcccggcacactgtctctgtctcccggcgaaagagccaccctgagctgcagagcatctcagagcgtgagcagcagctacctggcctggtatcagcagaagcccggccaagcccctagactgctgatctacggcgcaagcagcagagccaccggcatccccgacagattcagcggcagcggctctggcaccgacttcaccctgaccatctctcgactggagcctgaggacttcgccgtgtactattgccaacagtacggcaacccccccctgtacaccttcggccaaggaaccaaagtcgagatcaaggggggcggagggagcggcggaggcggaagcggcggtggtggttcggaggtacaactattggaatccggtggcggattagtgcagccgggcggcagcttaagactctcatgcgccgcgtcgggctttacctttagtagctatgccatgtcctgggttcggcaagcccccggcaaaggcctggagtgggtgtcagcgatcagcggcagcggaggtagcacctactacgccgacagcgtgaagggtagattcacgatcagcagagacaatagcaagaacaccctgtacctgcagatgaacagcctgagagccgaggacactgccgtatactactgcgccaaggtggccccctacttcgcccccttcgactactggggccaaggcacactggtcaccgtcagctcg

SEQ ID NO: 74 - DNA, 657 bp, synthetic construct
  source 1..657: mol_type = other DNA
gacgtgctgatgacacagacccccctgagcctgcccgtgagcctgggcgaccaagcaagcatcagctgcagaagctctcagagcatcgtgaacagcaacggcaacacctacctggagtggtacctgcagaagcctgggcagagccccaagctgctgatctacaaggtgagcaatcggttcagtggggtgcctgacagattcagtggttccggtagcggcaccgacttcaccctgaagatcagcagagtggaggccgaggacctgggcgtgtactactgcttccaaggcagccacgtgcccctgaccttcggggccggcaccaagctcgagatcaagagaactgtggccgcgccgtcagtgtttatcttccctccatcggatgaacagcttaagtccggcacggcgtctgtggtctgcctgctcaataacttttaccctagggaagctaaagtccaatggaaagtggataacgccctgcagtcaggaaacagccaggaatcggttaccgaacaggacagcaaggacagcacttactccttgtcgtcgactcttactctgagcaaggccgattacgagaagcacaaggtctacgcctgcgaggtcacccatcagggactctcgtccccggtgaccaaatccttcaatagaggcgaatgc

SEQ ID NO: 75 - protein, 328 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 76 - protein, 107 aa, synthetic construct [sequence omitted]

SEQ ID NO: 77 - protein, 112 aa, synthetic construct
DVLMTQTPLSLPVSLGDQASISCRSSQSIVNSNGNTYLEWYLQKPGQSPKLLIYKVSNRFSGVPDRFSGSGSGTDFTLKISRVEAEDLGVYYCFQGSHVPLTFGAGTKLEIK

SEQ ID NO: 78 - DNA, 363 bp, synthetic construct
  source 1..363: mol_type = other DNA
cagatccagttggtgcagtctggacctgagctgaagaagcctggagagacagtcaagatctcctgcaaggcttctggatatattttcacaaactatggaatgaactgggtgagacaggctccgggaaagggtttaaagtggatgggctggataaactcctacactggagaggcaacatatactgacgacctcaagggacggtttgccttctctttggaatcctctgccagcactgcctatttgcagatcaacaacctcaaaaatgaggacacggctacttatttctgtgcaagagagggatatggtgactacgggtactcctttgactactggggccaaggcaccactctcacagtctcctca

SEQ ID NO: 79 - DNA, 357 bp, synthetic construct
  source 1..357: mol_type = other DNA
cagatccagttggtgcagtctggacctgaactgaagaagcctggagagacagtcaagatctcctgcaaggcttctggttttaccttcacagactattcaatccactgggtgaagcaggctccaggaaagggtttaaagtggatgggctggaaacacactgaaagtggtgagtcaacatatgcagatgacttcaagggacggtttgtcttctctttggaaacctctgccagcactgcctatttgcagatcaacaacctcaaaaatgaggacacgtctacatatttctgtgctaggggggcgaactatggttccctacttgactactggggccaaggcaccactctcacagtctcctca

SEQ ID NO: 80 - DNA, 336 bp, synthetic construct
  source 1..336: mol_type = other DNA
gatgttttgatgacccaaactccactctccctgcctgtcagtcttggagatcaagcctccatctcttgcagatctagtcagagcattgtaaatagtaatggaaacacctatttagaatggtacctgcagaaaccaggccagtctccaaaactcctcatctacaaagtttccaaccgattttctggggtcccagacaggttcagtggcagtggatcagggacagatttcacactcaagatcagcagagtggaggctgaggatctgggagtttattactgctttcaaggttcacatgttccgctcacgttcggtgctgggaccaagctggagctgaaa

(19) United States
(12) Patent Application Publication
(10) Pub. No.: US 2026/0071003 A1
(43) Pub. Date: Mar. 12, 2026

(54) COMPOSITIONS AND METHODS FOR DIRECTING APOLIPOPROTEIN L1 TO INDUCE MAMMALIAN CELL DEATH

(71) Applicant: UNIVERSITY OF GEORGIA RESEARCH FOUNDATION, INC., Athens, GA (US)

(72) Inventors: Stephen L. HAJDUK, Athens, GA (US); Eric G. DEJESUS, Athens, GA (US); Michael J. CIPRIANO, Athens, GA (US)

(21) Appl. No.: 19/107,642

(22) PCT Filed: Sep. 1, 2023
(86) PCT No.: PCT/US2023/073334

§ 371 Date: Feb. 28, 2025

Related U.S. Application Data
(60) Provisional application No. 63/374,356, filed on Sep. 1, 2022.

(51) Int. Cl. C07K 16/30; A61K 38/00; A61P 35/00; C12N 15/11; C12N 15/85

(52) U.S. Cl. CPC C07K 16/3061; A61K 38/00; A61P 35/00; C12N 15/11; C12N 15/85; C07K 2317/24; C07K 2317/31; C07K 2317/565; C07K 2317/73

(57) ABSTRACT

Provided herein are compositions for increasing Apolipoprotein L1 (ApoL1) in target cells. The ApoL1 can be a recombinant protein or an endogenous protein optionally in a ApoL1-containing complex. Antibodies and other binding molecules that specifically bind, Apolipoprotein L1 (ApoL1) and Haptoglobin related protein (Hpr). In preferred embodiments, the antibodies and other molecules bind to an ApoL1-containing complex such as a Trypanosome Lytic Factor (TLF), preferably under physiological conditions. In preferred embodiments, the antibodies and antigen binding fragments are bispecific, trispecific, and multispecific molecules that can bind to the ApoL1-containing complex and further bind to a cell specific antigen. Methods of using such molecules to increase flux of ApoL1-containing complexes into target cells expressing the cell specific antigen are also provided. Such increase in ApoL-containing complexes can increase cell death. In preferred embodiments, the target cells are cancer cells such as blood cancer cells or solid tumor cells.

### CROSS-REFERENCE TO RELATED APPLICATIONS

This application claims the benefit of and priority to U.S. Provisional Application No. 63/374,356 filed Sep. 1, 2022, which is hereby incorporated by reference in its entirety.

### STATEMENT REGARDING FEDERALLY SPONSORED RESEARCH OR DEVELOPMENT

This invention was made with government support under grant number RO1-AI039033 awarded by the NIH. The government has certain rights in the invention. (37 CFR § 401.14 f (4)).

### REFERENCE TO SEQUENCE LISTING

The Sequence Listing submitted as a text file named “UGA_2022-004-02_PCT_ST26.xml”, created on Sep. 1, 2023, and having a size of 81,564 bytes is hereby incorporated by reference pursuant to 37 C.F.R. § 1.52(e)(5).

### FIELD OF THE INVENTION

The present invention relates to the field of targeted, induced cell death particularly by increasing accumulation of Apolipoprotein L1 (ApoL1) in target cells.

### BACKGROUND OF THE INVENTION

Active Multiple myeloma (MM) is an incurable malignancy of the plasma cells accounting for nearly 1.8% of all newly diagnosed cancers in the United States in 2021 (Siegel, et al., CA Cancer J Cin., 67:7-30 (2017)). It is characterized by the proliferation (>10%) of malignant, monoclonal plasma cells in bone marrow, along with hypercalcemia, renal failure, anemia, bone lytic lesions, (Mikhael, et al., Clin Lymphoma Myeloma Leuk., 20:1-7 (2020)) and claims the lives of over 12,000 people in the US each year (Siegel, et al., CA Cancer J Cin., 72:7-33 (2022)). Disease progression results from resistance to single treatment strategies including stem-cell transplantation, small molecule drugs, and biologics. Additionally, an increasing subset of patients, around 45,000 per year, are experiencing triple and quadruple refractory responses where all formerly used tactics become ineffective, termed Relapse Refractory Multiple Myeloma (RRMM), thereby emphasizing the need for new therapeutic concepts (Sonneveld, et al., Haematologica, 101:396-406 (2016)).

With an aging population, and better diagnostic capabilities, the number of reported MM cases is expected to increase in the coming years. The emergence of resistance to prior treatments requires many options for therapies to be available for further rounds of treatment. With each line of therapy, patient response varies, with 74% of the patients having a very good partial response at the first line of treatment, to only 11% after the fifth line of treatment (Sonneveld, et al., Haematologica, 101:396-406 (2016)). The time to progression (TTP) is the time from the start of a treatment until disease progression. TTP between treatment lines decreases with each line of treatment, from an 18-month TTP after the first line of treatment, 13 months at the second, 7 months at the third, and only 5-month TTP during subsequent treatment lines. With each line of treatment, an assessment must be made to determine what combination therapy can be used based on each patient's response during prior lines of treatment. It is these multiple lines of treatment with a breadth of therapies to choose from, which has allowed the survival rate to increase.

The advent of new treatment options has increased the survival rate, going from a 24% five-year survival rate in the 1980s to currently 50% with median survivals between 29 and 62 months (Wong, et al., Blood, 132 Supplement 1:4773 (2018). However, this increase in survival causes a great increase in costs associated with treating this disease due to multiple treatment regiments required after many rounds of relapse. Thus, there remains a need for additional therapeutic lines of treatment which have different mechanisms of action, especially for later lines of treatment when standard of care medication is not an option due to reduced effectiveness or decline in the health of the patient.

Thus, it is an object of the invention to provide alternative compositions and methods for the treatment of multiple myeloma and other cancers.

### SUMMARY OF THE INVENTION

Compositions and method of use thereof for increasing cell death of target cells in a mammalian subject, such as human, in need thereof. The methods typically include administering the subject an effective amount of a composition that increases Apolipoprotein L1 (ApoL1) (e.g., endogenous or exogenous ApoL1) in the target cells. Compositions that bind to both ApoL1 or ApoL1-containing complexes such as Trypanosome Lytic Factor (TLF), optionally TLF-1 and/or TLF-2, and a cell specific antigen are provided. Preferred compositions are bi- and multispecific antibodies having a first antigen binding fragment that binds to ApoL1 or an ApoL1-containing complex optionally a TLF and a second antigen binding fragment that binds to a cell specific antigen.

In other embodiments, the composition includes ApoL1 or a function fragment or variant thereof and a targeting moiety for a cell specific antigen. The ApoL1 or a function fragment or variant thereof conjugated or fused directly or indirectly the targeting moiety. In some embodiments, the composition includes a delivery vehicle, optionally liposomes or polymeric nanoparticles. The targeting moiety can be conjugated or fused to the delivery vehicle. Preferred targeting moieties are antibodies and antigen binding fragments.

The target cells can be mammalian or non-mammalian cells. The mammalian cells can be diseased (e.g., cancerous) or infected cells. The non-mammalian cells can be, for example, bacteria, fungi, or non-mammalian eukaryotic cells. The cells can be human cells.

The cell specific antigen can be specific for diseased cells. The diseased cells can be cancer cells such blood cancer cells and solid tumor cells. In some embodiments, the subject suffers from a disease caused by the target cells, and the composition is administered in an effective amount to treat the disease. Preferably, the cell specific antigen is not a trypanosome specific surface antigen and the subject does not have trypanosomiasis.

Also provided herein are antibodies and other binding molecules that specifically bind Apolipoprotein L1 (ApoL1) and Haptoglobin related protein (Hpr). In preferred embodiments, the antibodies and other molecules bind to ApoL1-containing complexes such as Trypanosome Lytic Factor (TLF). Preferably, the antibodies and other molecules bind to ApoL1-containing complexes such as TLF under physiological conditions including, but not limited to, endogenous complexes in vivo.

The antibody or antigen binding fragment can be or include an anti-ApoL1 antibody or antigen binding fragment including the three complementarity determining regions (CDRs) of the heavy chain variable domain of SEQ ID NO:24, or variants thereof with at least 70, 80, 90, or 95% sequence identity thereto, and the three complementarity determining regions (CDRs) of the light chain variable domain of SEQ ID NO:36 or SEQ ID NO:77, or variants thereof with at least 70, 80, 90, or 95% sequence identity thereto. In some embodiments, the heavy and light chain variable domain CDRs include:

(SEQ ID NO: 25)
TYAMS,
(SEQ ID NO: 26)
EISNGGLYTYYPDTVTG,
(SEQ ID NO: 27)
ENRNWYFDL,
(SEQ ID NO: 37)
RSSQSIVNSNGNTYLE,
and
(SEQ ID NO: 38)
KVSNRFS,
(SEQ ID NO: 39)
FQGSHVPLT,
or
variants thereof with at least 70, 80, 90, or 95% sequence
identity thereto;
(SEQ ID NO: 28)
GFTFSTYA,
(SEQ ID NO: 29)
ISNGGLYT,
(SEQ ID NO: 30)
IRENRNWYFDL,
(SEQ ID NO: 40)
QSIVNSNGNTY,
KVS,
and
(SEQ ID NO: 39)
FQGSHVPLT,
or
variants thereof with at least 70, 80, 90, or 95% sequence
identity thereto;
or
(SEQ ID NO: 31)
GFTFSTY,
(SEQ ID NO: 32)
SNGGLY,
(SEQ ID NO: 27)
ENRNWYFDL,
(SEQ ID NO: 37)
RSSQSIVNSNGNTYLE,
(SEQ ID NO: 38)
KVSNRFS,
and
(SEQ ID NO: 39)
FQGSHVPLT,
or

variants thereof with at least 70, 80, 90, or 95% sequence identity thereto.

In some embodiments, the antibody or antigen binding fragment includes a heavy chain variable domain including the amino acid sequence of SEQ ID NO:24 or a variant thereof with at least 70, 80, 90, or 95% sequence identity thereto and a light chain variable domain including the amino acid sequence of SEQ ID NO:36 or SEQ ID NO:77 or a variant thereof with at least 70, 80, 90, or 95% sequence identity thereto.

The antibody or antigen binding fragment can be or include an anti-Hpr antibody or antigen binding fragment including the three complementarity determining regions (CDRs) of the heavy chain variable domain of SEQ ID NO:3, or variants thereof with at least 70, 80, 90, or 95% sequence identity thereto, and the three complementarity determining regions (CDRs) of the light chain variable domain of SEQ ID NO:14, or variants thereof with at least 70, 80, 90, or 95% sequence identity thereto. In some embodiments, the heavy and light chain variable domain CDRs include:

(SEQ ID NO: 4)
NYGMN,
(SEQ ID NO: 5)
WINSYTGEATYTDDLKG,
(SEQ ID NO: 6)
EGYGDYGYSFDY,
(SEQ ID NO: 16)
RATKNIYTYLA,
(SEQ ID NO: 17)
NAKTLAE,
and
(SEQ ID NO: 18)
QHHYGTPRT,
or
variants thereof with at least 70, 80, 90, or 95% sequence
identity thereto;
(SEQ ID NO: 7)
GYIFTNYG,
(SEQ ID NO: 8)
INSYTGEA,
(SEQ ID NO: 9)
AREGYGDYGYSFDY,
(SEQ ID NO: 19)
KNIYTY,
NAK,
and
(SEQ ID NO: 18)
QHHYGTPRT,
or
variants thereof with at least 70, 80, 90, or 95% sequence
identity thereto;
or
(SEQ ID NO: 10)
GYIFTNY,
(SEQ ID NO: 11)
NSYTGE,
(SEQ ID NO: 6)
EGYGDYGYSFDY,
(SEQ ID NO: 16)
RATKNIYTYLA,
(SEQ ID NO: 17)
NAKTLAE,
and
(SEQ ID NO: 18)
QHHYGTPRT,
or
variants thereof with at least 70, 80, 90, or 95% sequence
identity thereto.

In some embodiments, the antibody or antigen binding fragment includes a heavy chain variable domain including the amino acid sequence of SEQ ID NO:3 or a variant thereof with at least 70, 80, 90, or 95% sequence identity thereto and a light chain variable domain including the amino acid sequence of SEQ ID NO: 14 or a variant thereof with at least 70, 80, 90, or 95% sequence identity thereto.

The antibody or antigen binding fragment can be or include an anti-Hpr antibody or antigen binding fragment including the three complementarity determining regions (CDRs) of the heavy chain variable domain of SEQ ID NO:56, or variants thereof with at least 70, 80, 90, or 95% sequence identity thereto, and the three complementarity determining regions (CDRs) of the light chain variable domain of SEQ ID NO:65, or variants thereof with at least 70, 80, 90, or 95% sequence identity thereto. In some embodiments, the heavy and light chain variable domain CDRs include:

(SEQ ID NO: 57)
DYSIH,
(SEQ ID NO: 58)
WKHTESGESTYADDFKG,
(SEQ ID NO: 59)
GANYGSLLDY,
(SEQ ID NO: 66)
RASKSVSTSGYSYMH,
(SEQ ID NO: 67)
LASNLES,
(SEQ ID NO: 68)
QHNRELPLT,
or
variants thereof with at least 70, 80, 90, or 95% sequence
identity thereto;
(SEQ ID NO: 60)
GFTFTDYS,
(SEQ ID NO: 61)
KHTESGES,
(SEQ ID NO: 62)
ARGANYGSLLDY,
(SEQ ID NO: 69)
KSVSTSGYSY,
LAS,
(SEQ ID NO: 68)
QHNRELPLT,
or
variants thereof with at least 70, 80, 90, or 95% sequence
identity thereto;
or
(SEQ ID NO: 63)
GFTFTDY,
(SEQ ID NO: 64)
HTESGE,
(SEQ ID NO: 59)
GANYGSLLDY,
(SEQ ID NO: 66)
RASKSVSTSGYSYMH,
(SEQ ID NO: 67)
LASNLES,
(SEQ ID NO: 68)
QHNRELPLT,
or
variants thereof with at least 70, 80, 90, or 95% sequence
identity thereto.

In some embodiments, the antibody or antigen binding fragment includes a heavy chain variable domain including the amino acid sequence of SEQ ID NO:56 or a variant thereof with at least 70, 80, 90, or 95% sequence identity thereto and a light chain variable domain including the amino acid sequence of SEQ ID NO:65 or a variant thereof with at least 70, 80, 90, or 95% sequence identity thereto.

The antibody or antigen binding fragment can include one or more constant domains from an immunoglobulin constant region (Fc), optionally wherein the constant domains are human constant domains. In some embodiments, the constant domains are IgA, IgD, IgE, IgG or IgM constant domains. In some embodiments, the antibody or antigen binding fragment includes one or more human IgG constant domains, optionally IgG1, IgG2, IgG3, or IgG4 domains. In some embodiments, the antibody or antigen binding fragment is not a mouse IgG1 or IgG2a.

The antibody or antigen binding fragment can be detectably labeled or include a conjugated toxin, drug, receptor, enzyme, receptor ligand.

In some embodiments, the antibody or antigen binding fragment is a monoclonal antibody, a human antibody, a chimeric antibody, or a humanized antibody. The antibody or antigen binding fragment can be a bispecific, trispecific or multispecific antibody. In some embodiments, variants are of the provided sequences are humanized forms of the sequence.

In preferred embodiments, the anti-ApoL1 and/or anti-Hpr antibody or antigen binding fragment is in a bispecific, trispecific or multispecific antibody includes a second (or third or more) antigen binding fragment that binds to a cell specific antigen. Thus provided are bispecific, trispecific or multispecific antibodies having one or more antigen binding fragments that binds to ApoL1-containing complexes such as TLF and a second (third or more) antigen binding fragment that binds to a cell specific antigen. In some embodiments, the cell specific antigen is a cancer or tumor antigen. The cancer antigen can be a blood cancer antigen, optionally selected from BCMA, PD-L1/B7-HA/CD247, CTLA4, CD38, CD319/SLAMF-7, TNFRSF17/BCMA, SYND1/CD138, CD229, CD47, CD123/IL3-RA, CD19, CD20, CD22, FcRH5, GPRC5D, and CLL-1. Thus, in some embodiments, the composition includes an antibody or antigen binding fragment that binding to BCMA, PD-L1/B7-HA/CD247, CTLA4, CD38, CD319/SLAMF-7, TNFRSF17/BCMA, SYND1/CD138, CD229, CD47, CD123/IL3-RA, CD19, CD20, CD22, FcRH5, GPRC5D, and CLL-1.

In other embodiments, the tumor antigen is a pancreatic cancer antigen, optionally selected from Claudin 18.2, MUC1, Mesothelin (MSLN), and Myoferlin (MYOF). Thus, in some embodiments, the bi- or multispecific antibody includes an antigen binding fragment that specifically binds to Claudin 18.2, MUC1, Mesothelin (MSLN), and Myoferlin (MYOF).

In other embodiments, the tumor antigen is a melanoma cancer antigen, optionally PMEL17. Thus, in some embodiments, the bi- or multispecific antibody includes an antigen binding fragment that specifically binds to PMEL17.

In some embodiments, the second antigen binding fragment is an anti-BCMA antigen binding fragment including the three CDRs of the heavy chain variable domain of SEQ ID NO:41 or variant thereof with at least 70, 80, 90, or 95% sequence identity thereto and the three CDRs of the light chain variable domain of SEQ ID NO:42 or variant thereof with at least 70, 80, 90, or 95% sequence identity thereto. For example, the CDRs can be, CDR1H: SYAMS (SEQ ID NO:43) or variant thereof with at least 70, 80, 90, or 95% sequence identity thereto, CDR2H: AISGSGGSTYYADSVKG (SEQ ID NO:44) or variant thereof with at least 70, 80, 90, or 95% sequence identity thereto, CDR3H: VAPYFAPFDY (SEQ ID NO:45) or variant thereof with at least 70, 80, 90, or 95% sequence identity thereto, CDR1L: RASQSVSSSYLA (SEQ ID NO:46) or variant thereof with at least 70, 80, 90, or 95% sequence identity thereto, CDR2L: GASSRAT (SEQ ID NO:47) or variant thereof with at least 70, 80, 90, or 95% sequence identity thereto, and CDR3L: QQYGNPPLYT (SEQ ID NO:48) or variant thereof with at least 70, 80, 90, or 95% sequence identity thereto. In some embodiments, the second antigen binding fragment includes a heavy chain variable domain including the amino acid sequence of SEQ ID NO:41 or variant thereof with at least 70, 80, 90, or 95% sequence identity thereto and a light chain variable domain including the amino acid sequence of SEQ ID NO:42 or variant thereof with at least 70, 80, 90, or 95% sequence identity thereto.

In some embodiments, the antibody or antigen binding fragment includes the amino acid sequence of SEQ ID NO:71 or variant thereof with at least 70, 80, 90, or 95% sequence identity thereto and/or the amino acid sequence of SEQ ID NO:72 or variant thereof with at least 70, 80, 90, or 95% sequence identity thereto. In specific embodiments, the antibody or antigen binding fragment includes the amino acid sequences of SEQ ID NO:71 and SEQ ID NO:72.

In particular exemplary structures, the antibody is an anti-ApoL1, anti-cell specific antigen IgG1-scFv bispecific chimeric antibody, or an anti-Hpr, anti-cell specific antigen IgG1-scFv bispecific chimeric antibody, optionally having the structure of FIG. 8.

In a specific embodiments, the bispecific antibody has the structure of FIG. 8 formed by two copies each of the amino acid sequences of SEQ ID NO:71 and SEQ ID NO:72, e.g., upon co-expression of nucleic acids encoding the amino acid sequences of SEQ ID NO:71 (e.g., SEQ ID NO:73) and SEQ ID NO:72 (e.g., SEQ ID NO:74).

Nucleic acids including DNA and RNA encoding the disclosed antibodies and antigen binding fragments are also provided. The nucleic acids can be operably linked to an expression control sequence. Expression vectors, the encoding sequences, and cells, e.g., bacterial and mammalian cells, transformed with the nucleic acids and vectors are also provided.

Methods of forming immunocomplexes by contacting the disclosed antibodies and antigen binding fragments with an ApoL1-containing complex such as TLF, and the immunocomplexes optionally formed therefrom and optionally further complexed with the surface of a cell are also provided. It is believed that such immune complexes, when transported into cells, will lead to increased cells death. Thus, methods of inducing cell death by contacting target cells with the immunocomplexes are provided. The contacting can occur in vitro or in vivo.

Pharmaceutical compositions including an effective amount of the disclosed antibodies and antigen binding fragments are also provided.

Methods of treating cancer are also provided and can include administering the subject an effective amount of the antibody or antigen binding fragment. Preferrable, the antibodies and antigen binding fragments used in such methods include a second (or more) antigen binding fragment that binds to a cell specific antigen, such as tumor antigen and enhances delivery of ApoL1-containing complexes such as TLF to the cells expressing the antigen. See, e.g., FIG. 9. The cancer can be a blood cancer, such as multiple myeloma, leukemia (e.g., chronic lymphocytic leukemia, acute myeloid leukemia, acute lymphoblastic leukemia), non-Hodgkin lymphoma, Hodgkin lymphoma, myelodysplastic syndromes (MDS), myeloproliferative neoplasms (MPNs) (or a subcategory thereof, e.g., essential thrombocythemia (ET), myelofibrosis (MF) and polycythemia vera (PV), amyloidosis, Waldenstrom macroglobulinemia, or aplastic anemia, or a solid cancer.

### BRIEF DESCRIPTION OF THE DRAWINGS

FIGS. 1A-IC are illustrations of select high-density lipoprotein (HDL) complexes containing Apolipoprotein L1 (ApoL1). Circulating innate factors in all human beings, consistent with all HDL particles, they contain: a hydrophobic core and cholesterol. Trypanosome Lytic Factor 1 (TLF-1) (FIG. 1A) is lipid rich and has the proteins ApoL1, Hpr (with bound Hb), and ApoA1. Trypanosome Lytic Factor 2 (TLF-2) (FIG. 1B) is lipid poor and contains a unique IGM in addition to TLF-1 proteins. HDL complexes may also contain ApoL1 in the absence of TLF (FIG. 1C). Non-lethal at physiological concentrations, a 10× increase of TLF-1 physiological concentrations leads to indiscriminate cell lethality.

FIG. 2 is a plot showing TLF-1 binding to HEK293 cells. Putative binding estimates were attempted based on half-maximal binding at 3° C. with varying concentrations of Alexa-488 TLF-1. No saturation was obtained. 20,000 cells per assay were analyzed in triplicate through flow cytometry.

FIG. 3A is a series of images showing TLF-1 uptake (20 mg/ml) in HEK293 cells for two hours (images captured by Amnis). FIG. 3B is a plot quantifying the images of FIG. 3A. Pixel density was calculated by ImageStream 6.0 software and plotted as a percentage of max pixel density. FIG. 3C is a series of images showing colocalization of TLF and lysotracker in live HEK293 cells. To investigate TLF update in mammalian cells, HEK293 cells were incubated with Alexa Fluor 488-conjugated TLF (AF488 TLF) and imaged via ImageStream. AF488 TLF is endocytosed into vesicles inside the HEK293 cells. The quantification of the signal intensity indicates the maximum pixel intensity signifying TLF is taken into the cell at 37° C. update. No cell surface binding is detectible when the cells are held at 3° C. (FIG. 3B). FIG. 3D is a plot showing competition with 2, 10, 50, and 100× unlabeled competitor (non-lytic HDL by mass or HP-1 by molecular weight) in low temperature binding assay. FIG. 3E is a plot showing the time course of TLF-1 uptake in HEK293 cells. Median intensity of TLF-1 was measure and quantified by Flowlo 9.6.4 software. Data points are indicative of 20,000 cells per point.

FIG. 4A is a plot showing the viability of HEK293 cells over time (days) following incubation with control (No TLF), non-lytic HDLs which do not contain ApoL1 (NLHDL), 10 μg/ml TLF, or 75 μg/ml of TLF. FIG. 4B is a series of microscopy images showing HEK293 treated with no TLF (left), non-lytic HDL (75p g/ml) (center), and TLF (75p g/ml) (right). FIG. 4C is a bar graph showing the percent reduction in growth of CCL-155 multiple myeloma cells following incubation with high concentrations of purified human TLF (1.26 float fraction, a sub fractionation of human serum that contains TLF). Increases in high concentration TLF/HDL fraction result in reduced viability of cells are measure by CellTiterGlo. *** 25% reduction at 4.96 mg/ml total protein content. FIGS. 4D-G are plots that show exogenously added ApoL1 reduces growth of multiple mammalian cell lines. Cell lines chosen represent various cancer models, CCL-155 (RPMI 8226): multiple myeloma (FIG. 4D), PANC-1: pancreatic (FIG. 4E), A375: melanoma (FIG. 4F), HT144: melanoma (FIG. 4G). Specified cell lines were incubated with recombinant ApoL1 for three or four days as indicated. Cell viability was assayed on day four using CellTiter-Glo. Data shows a dose dependent effect of recombinant ApoL1 on cell growth. An LD50 was calculated using Quest Graph EC50 Calculator using a four-parameter model. The results are as follows: CCL-155: 20.2 μg/mL, PANC-1: 43 μg/mL, A375: 27.5 μg/mL, HT144: 32.8 μg/mL. Points represent each replicate, with a dashed line representing the mean. Error bars represent the standard deviation. FIG. 4H is a plot showing the number of viable RPMI 8226 (CCL-155) cells following incubation with media containing purified human TLF after immunoprecipitation using various concentrations of anti-ApoL1 (μg/ml) antibody. FIG. 4I is a plot showing the number of viable RPMI 8226 (CCL-155) cells following incubation with media containing purified human TLF after immunoprecipitation using various concentrations of anti-Hpr (μg/ml) antibody.

FIGS. 5A-5F show the characterization of recombinant anti-Hpr antibody. FIGS. 5A and 5D are stain free images of total protein (TLF and recombinant ApoL1) electrophoresed under non-reducing (FIG. 5A) and reducing (FIG. 5D) conditions. FIGS. 5B and 5E are images of Western blots utilizing recombinant anti-Hpr antibody under non-reducing (FIG. 5B) and reducing (FIG. 5E) conditions. FIGS. 5C and 5F are images of Western blots utilizing ascites Prot-G purified anti-Hpr antibody under non-reducing (FIG. 5C) and reducing (FIG. 5F) conditions. FIG. 5G is a dot blot (native) showing recombinant and ascites purified anti-Hpr antibodies binding to recombinant ApoL1.

FIGS. 6A-6G show the characterization of recombinant anti-ApoL1 antibody. FIGS. 6A and 6D are stain free images of total protein (TLF and recombinant ApoL1) electrophoresed under non-reducing (FIG. 5A) and reducing (FIG. 6D) conditions. FIGS. 6B and 6E are images of Western blots utilizing recombinant anti-ApoL1 antibody under non-reducing (FIG. 6B) and reducing (FIG. 6E) conditions. FIGS. 6C and 6F are images of Western blots utilizing ascites Prot-G purified anti-ApoL1 antibody under non-reducing (FIG. 6C) and reducing (FIG. 6F) conditions. FIG. 6G is a dot blot (native) showing recombinant and ascites purified anti-ApoL1 antibodies binding to recombinant ApoL1.

FIGS. 7A-7C show the characterization of recombinant anti-BCMA scFv. FIG. 7A is a stain free image of total protein (nr- and r-BCMA). FIGS. 7B and 7C are images of Western blots utilizing anti-BCMA clone 17A5 scFv (SEQ ID NOL51) (FIG. 7B) and anti-BCMA intact monoclonal antibody (RnD Systems Cat. MAB1931) (FIG. 7C).

FIG. 8 is an illustration of an exemplary anti-ApoL1, anti-BCMA antibody having a Fab portion with the heavy and light chain variable regions of recombinant clone 13.11 (anti-ApoL1) having the sequences provided in Example 6, and an anti-BCMA as an ScFv (SEQ ID NO:51) provided in Example 7 fused to the Heavy Chain C-terminus of a human IgG1. See also, Example 8.

FIGS. 9A-9B show the binding of a bispecific antibody designed according to FIG. 8/Example 8 to BCMA and Apolipoprotein L1. FIG. 9A shows steps involved in bridging ELISA assay to assess the binding capacity of the bispecific antibody (bsAb) between recombinant variations of ApoL1 and BCMA. FIG. 9B shows the bsAb successfully binding to immobilized forms of both ligands (i.e., forming a bridge between the ligands).

FIGS. 10A-10B illustrate ApoL1-induced cell death processes. Using the Promega RealTime-Glo Annexin V Apoptosis and Necrosis assay, RPMI 8226 Multiple Myeloma cells were analyzed in the presence of 3.1 μg/ml ApoL1+15 μg/ml bsAb, 15 μg/ml bsAb only, 3.1 μg/ml ApoL1 only, and cells alone (“Cell only”). Within four hours, cellular apoptotic signals were observed to increase between 50-127% above Cell only levels with the highest levels measured when bsAb was added (FIG. 10A). Within the same RealTime assay, necrosis was measured under the same parameters: 3.1 μg/ml ApoL1+15 μg/ml bsAb, 15 μg/ml bsAb only, 3.1 μg/ml ApoL1 only, and Cells alone. From 4 to 16 hours, when compared to Cell only, an increase in necrotic signaling above ApoL1 alone was measured in lines containing bsAb. At 16 hours, this effect peaked at 67% for cells containing ApoL1 and bsAb. Points on the graphs are shown as the mean of 4 replicates with error bars representing the standard deviation. Data was normalized by setting baseline to Cell only, then expressing data as an average mean percentage difference compared to the baseline (FIG. 10B).

FIGS. 11A-11E demonstrate binding of ApoL1-BCMA-bsAb and ApoL1-488 to RPMI8226 (CCL-155) Multiple myeloma cells. FIG. 11A is a pair of scatter plots showing a gating strategy for selecting in focus cells (Gradient RMS) and cells of the correct aspect ratio (Aspect Ratio) to select for single cells and exclude speed beads (Aspect Ratio). Secondary gating selects cells of the correct size to further remove speed beads, and cells which exclude Zombie NIR dye, a dead cell indicator. FIG. 11B is a plot and chart showing labeled bsAb vs. labeled isotype control. Cells passing through the gating strategy were measured for 488 excitation/525 emission intensity to measure bound 488-bsAb or bound 488-IgG1 isotype control. FIG. 11C is a plot and chart showing the results of unlabeled bsAb competition assay to help determine if bsAb is labeling specifically. Additional non-labeled bsAb was added to the reaction to act as a competitive inhibitor. Increased amounts of unlabeled bsAb shows a reduction in bound Alexa-488 intensity. CV is defined as the coefficient of variation calculated as 100×(standard deviation/mean). FIGS. 11D-11E illustrate secondary binding of labeled ApoL1 after binding of recombinant ApoL1-BCMA-bsAb to multiple myeloma cells RPMI 8226 (ATCC CCL-155). FIG. 11D is a pair of scatter plots showing a gating strategy for selecting in focus cells (Gradient RMS) and cells of the correct aspect ratio (Aspect Ratio) to select for single cells and exclude speed beads (Aspect Ratio). Secondary gating selects cells of the correct size to further remove speed beads, and cells which exclude Zombie NIR dye, a dead cell indicator. FIG. 11E is a plot and chart showing measurements of the intensity of ApoL1-488 bound to cell samples previously incubated with ApoL1-BCMA-bsAb or with human IgG1 isotype control. CV is defined as the coefficient of variation calculated as 100× (standard deviation/mean).

FIG. 12 is an overview of an exemplary proposed therapeutic method and ensuing mechanism. (A) Bispecific antibodies (bsAb) (e.g., for TLF and a target cell marker such as BCMA) are injected into a subject, (B) bind to endogenous TLF and (C) link it to the surface of target cells (e.g., myeloma cells). (D) Target cell marker (e.g., BCMA) mediated endocytosis internalizes immune complex, increasing the intracellular concentration of TLF with the lysosome above the viability threshold and ultimately resulting in (E) target cell death.

### DETAILED DESCRIPTION OF THE INVENTION

### I. Definitions

As used herein, the term “binds” in reference to the interaction of a binding protein and an antigen means that the interaction is dependent upon the presence of a particular structure (e.g., an antigenic determinant or epitope) on the antigen. For example, a binding protein recognizes and binds to a specific antigen structure rather than to antigens generally. For example, if a binding protein binds to epitope “A”, the presence of a molecule containing epitope “A” (or free, unlabeled “A”), in a reaction containing labeled “A” and the binding protein, will reduce the amount of labeled “A” bound to the binding protein.

As used herein, a molecule is said to be able to “immunospecifically bind” a second molecule if such binding exhibits the specificity and affinity of an antibody to its cognate antigen. Antibodies are said to be capable of “immunospecifically binding” to a target region or conformation (“epitope”) of an antigen (and in particular, an antigen of Hpr or ApoL1) if such binding involves the antigen recognition site of the immunoglobulin molecule. An antibody that immunospecifically binds to a particular antigen may bind to other antigens with lower affinity if the other antigen has some sequence or conformational similarity that is recognized by the antigen recognition site as determined by, e.g., immunoassays, BIACOREC assays, or other assays known in the art, but would not bind to a totally unrelated antigen.

Preferably, however, antibodies (and their antigen binding fragments) will not cross-react with other antigens. Antibodies may also bind to other molecules in a way that is not immunospecific, such as to FcR receptors, by virtue of binding domains in other regions/domains of the molecule that do not involve the antigen recognition site, such as the Fc region.

The term “substantially,” as used in the context of binding or exhibited effect, is intended to denote that the observed effect is physiologically or therapeutically relevant. Similarly, a molecule is said to have substantially the same immunospecificity and/or characteristic as another molecule, if such immunospecificities and characteristics are greater than 60% identical, greater than 70% identical, greater than 75% identical, greater than 80% identical, greater than 85% identical, greater than 90% identical, greater than 95% identical, or greater than 97% identical).

As used herein, the term “antibody” is intended to denote an immunoglobulin molecule that possesses a “variable region” antigen recognition site. The term antibody includes monoclonal antibodies, multi-specific antibodies, human antibodies, humanized antibodies, synthetic antibodies, chimeric antibodies, camelized antibodies (See e.g., Muyldermans et al., 2001, Trends Biochem. Sci. 26:230; Nuttall et al., 2000, Cur. Pharm. Biotech. 1:253; Reichmann and Muyldermans, 1999, J Immunol. Meth. 231:25; International Publication Nos. WO 94/04678 and WO 94/25591; U.S. Pat. No. 6,005,079), single-chain Fvs (scFv) (see, e.g., see Pluckthun in The Pharmacology of Monoclonal Antibodies, vol. 113, Rosenburg and Moore eds. Springer-Verlag, New York, pp. 269-315 (1994)), single chain antibodies, disulfide-linked Fvs (sdFv), intrabodies, and anti-idiotypic (anti-Id) antibodies (including, e.g., anti-Id and anti-anti-Id antibodies to the disclosed antibodies). In particular, such antibodies include immunoglobulin molecules of any type (e.g., IgG, IgE, IgM, IgD, IgA and IgY), class (e.g., IgG1, IgG2, IgG3, IgG4, IgAQ1 and IgA2) or subclass.

The term “variable region” is intended to distinguish such domain of the immunoglobulin from domains that are broadly shared by antibodies (such as an antibody Fc domain). The variable region refers to the portions of the light and/or heavy chains of an antibody as defined herein that specifically binds to an antigen and, for example, includes amino acid sequences of CDRs; i.e., CDR1, CDR2, and CDR3, and framework regions (FRs). For example, the variable region can include three or four FRs (e.g., FR1, FR2, FR3 and optionally FR4) together with three CDRs. VH refers to the variable region of the heavy chain. VL refers to the variable region of the light chain. The variable region includes a “hypervariable region” whose residues are responsible for antigen binding.

The hypervariable region includes amino acid residues from a “Complementarity Determining Region” or “CDR” (e.g., typically at approximately residues 24-34 (L1), 50-56 (L2) and 89-97 (L3) in the light chain variable domain and at approximately residues 27-35 (H1), 50-65 (H2) and 95-102 (H3) in the heavy chain variable domain according to Kabat; Kabat et al., Sequences of Proteins of hmmunological Interest, 5th Ed. Public Health Service, National Institutes of Health, Bethesda, MD. (1991)) and/or those residues from a “hypervariable loop” (e.g., residues 26-32 (L1), 50-52 (L2) and 91-96 (L3) in the light chain variable domain and 26-32 (HI), 53-55 (H2) and 96-101 (H3) in the heavy chain variable domain according to Chothia; Chothia and Lesk, 1987, J. Mol. Biol. 196:901-917). Conventions that include corrections or alternate numbering systems for variable domains include not only Kabat and Chothia, but also IMGT (Lefranc, et al. (2003), Dev Comp Immunol 27: 55-77), Chothia (Chothia C, Lesk A M (1987), J Mal Biol 196: 901-917; Chothia, et al. (1989), Nature 342: 877-883) and AHo (Honegger A, Pluckthun A (2001) J Mol Biol 309: 657-670). For convenience, examples of binding proteins of the present disclosure may also be labelled according to Kabat, Chothia, or IMGT. These examples are expressly indicated as such.

“Framework Region” or “FR” residues are those variable domain residues other than the hypervariable region residues as herein defined.

As used herein, the term “antigen binding fragment” of an antibody refers to one or more portions of an antibody that contain the antibody's Complementarity Determining Regions (“CDRs”) and optionally the framework residues that include the antibody's “variable region” antigen recognition site, and exhibit an ability to immunospecifically bind antigen. Such fragments include Fab, Fab′, F(ab′)2 and Fv fragments; diabodies; linear antibodies; single-chain antibody molecules and multispecific antibodies formed from antibody fragments, and mutants thereof, naturally occurring variants, and fusion proteins including the antibody's “variable region” antigen recognition site and a heterologous protein (e.g., a toxin, an antigen recognition site for a different antigen, an enzyme, a receptor or receptor ligand, etc.). For example, the term antigen binding fragment may be used to refer to recombinant single chain Fv fragments (scFv) as well as divalent (di-scFv) and trivalent (tri-scFV) forms thereof. Such fragments can be produced via various methods known in the art.

The term “constant region” as used herein, refers to a portion of heavy chain or light chain of an antibody other than the variable region. In a heavy chain, the constant region generally includes a plurality of constant domains and a hinge region, e.g., an IgG constant region includes the following linked components, a constant heavy CHI, a linker, a CH2 and a CH3. In a heavy chain, a constant region includes a Fc. In a light chain, a constant region generally include one constant domain (a CL 1).

The term “fragment crystalizable” or “Fc” or “Fc region” or “Fc portion” (which can be used interchangeably herein) refers to a region of an antibody including at least one constant domain and which is generally (though not necessarily) glycosylated and which is capable of binding to one or more Fc receptors and/or components of the complement cascade. The heavy chain constant region can be selected from any of the five isotypes: α, δ, ε, γ, or μ. Exemplary heavy chain constant regions are gamma 1 (IgG1). gamma 2 (IgG2) and gamma 3 (IgG3), or hybrids thereof.

A “constant domain” is a domain in an antibody the sequence of which is highly similar in antibodies/antibodies of the same type, e.g., IgG or IgM or IgE. A constant region of an antibody generally includes a plurality of constant domains, e.g., the constant region of γ, α or δ heavy chain include two constant domains.

The terms “full-length antibody”, “intact antibody” or “whole antibody” are used interchangeably to refer to an antibody in its substantially intact form, as opposed to an antigen binding fragment of an antibody. Specifically, whole antibodies include those with heavy and light chains including an Fc region. The constant domains may be wild-type sequence constant domains (e.g., human wild-type sequence constant domains) or amino acid sequence variants thereof.

A “chimeric antibody” is a molecule in which different portions of the antibody are derived from different immunoglobulin molecules such as antibodies having a variable region derived from a non-human antibody and a human immunoglobulin constant region. Methods for producing chimeric antibodies are known in the art. See e.g., Morrison, 1985, Science 229:1202; Oi et al., 1986, BioTechniques 4:214; Gillies et al., 1989, J. Immunol. Methods 125:191-202; and U.S. Pat. Nos. 6,311,415, 5,807,715, 4,816,567, and 4,816,397. Chimeric antibodies including one or more CDRs from a non-human species and framework regions from a human immunoglobulin molecule can be produced using a variety of techniques known in the art including, for example, CDR-grafting (EP 239,400; International Publication No. WO 91/09967; and U.S. Pat. Nos. 5,225,539, 5,530,101, and 5,585,089), veneering or resurfacing (EP 592,106; EP 519,596; Padlan, 1991, Molecular Immunology 28(4/5):489-498; Studnicka et al., 1994, Protein Engineering 7:805; and Roguska et al., 1994, Proc. Natl. Acad. Sci. USA 91:969), and chain shuffling (U.S. Pat. No. 5,565,332).

As used herein, the term “humanized antibody” refers to an immunoglobulin including a human framework region and one or more CDR's from a non-human (usually a mouse or rat) immunoglobulin. The non-human immunoglobulin providing the CDR's is called the “donor” and the human immunoglobulin providing the framework is called the “acceptor.”

As used herein, the term “fragment” refers to a peptide or polypeptide including an amino acid sequence of at least 5 contiguous amino acid residues, at least 10 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino residues, at least 70 contiguous amino acid residues, at least 80 contiguous amino acid residues, at least 90 contiguous amino acid residues, at least 100 contiguous amino acid residues, at least 125 contiguous amino acid residues, at least 150 contiguous amino acid residues, at least 175 contiguous amino acid residues, at least 200 contiguous amino acid residues, or at least 250 contiguous amino acid residues.

As used herein, the term “fusion protein” refers to a polypeptide formed by the joining of two or more polypeptides through a peptide bond formed between the amino terminus of one polypeptide and the carboxyl terminus of another polypeptide or through linking of one polypeptide to another through reactions between amino acid side chains (for example disulfide bonds between cysteine residues on each polypeptide). The fusion protein can be formed by the chemical coupling of the constituent polypeptides or it can be expressed as a single polypeptide from a nucleic acid sequence encoding the single contiguous fusion protein. Fusion proteins can be prepared using conventional techniques in molecular biology to join the two genes in frame into a single nucleic acid sequence, and then expressing the nucleic acid in an appropriate host cell under conditions in which the fusion protein is produced.

As used herein, the term “variant” refers to a polypeptide or polynucleotide that differs from a reference polypeptide or polynucleotide, but retains essential properties. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more modifications (e.g., substitutions, additions, and/or deletions). A substituted or inserted amino acid residue may or may not be one encoded by the genetic code. A variant of a polypeptide may be naturally occurring such as an allelic variant, or it may be a variant that is not known to occur naturally.

Modifications and changes can be made in the structure of the polypeptides of the in disclosure and still obtain a molecule having similar characteristics as the polypeptide (e.g., a conservative amino acid substitution). For example, certain amino acids can be substituted for other amino acids in a sequence without appreciable loss of activity. Because it is the interactive capacity and nature of a polypeptide that defines that polypeptide's biological functional activity, certain amino acid sequence substitutions can be made in a polypeptide sequence and nevertheless obtain a polypeptide with like properties.

In making such changes, the hydropathic index of amino acids can be considered. The importance of the hydropathic amino acid index in conferring interactive biologic function on a polypeptide is generally understood in the art. It is known that certain amino acids can be substituted for other amino acids having a similar hydropathic index or score and still result in a polypeptide with similar biological activity. Each amino acid has been assigned a hydropathic index on the basis of its hydrophobicity and charge characteristics. Those indices are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is believed that the relative hydropathic character of the amino acid determines the secondary structure of the resultant polypeptide, which in turn defines the interaction of the polypeptide with other molecules, such as enzymes, substrates, receptors, antibodies, antigens, and cofactors. It is known in the art that an amino acid can be substituted by another amino acid having a similar hydropathic index and still obtain a functionally equivalent polypeptide. In such changes, the substitution of amino acids whose hydropathic indices are within ±2 is preferred, those within ±1 are particularly preferred, and those within ±0.5 are even more particularly preferred.

Substitution of like amino acids can also be made on the basis of hydrophilicity, particularly where the biological functional equivalent polypeptide or peptide thereby created is intended for use in immunological embodiments. The following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); proline (−0.5±1); threonine (−0.4); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent polypeptide. In such changes, the substitution of amino acids whose hydrophilicity values are within ±2 is preferred, those within I are particularly preferred, and those within ±0.5 are even more particularly preferred.

As outlined above, amino acid substitutions are generally based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include (original residue: exemplary substitution): (Ala: Gly, Ser), (Arg: Lys), (Asn: Gln, His), (Asp: Glu, Cys, Ser), (Gln: Asn), (Glu: Asp), (Gly: Ala), (His: Asn, Gln), (Ile: Leu, Val), (Leu: Ile, Val), (Lys: Arg), (Met: Leu, Tyr), (Ser: Thr), (Thr: Ser), (Tip: Tyr), (Tyr: Trp, Phe), and (Val: Ile, Leu). Embodiments of this disclosure thus contemplate functional or biological equivalents of a polypeptide as set forth above. In particular, embodiments of the polypeptides can include variants having about 50%, 60%, 70%, 80%, 90%, 95%, 96%, 97%, 98%, 99%, or more sequence identity to the polypeptide of interest.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill of those practicing in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared.

As used herein, the term “tumor” or “neoplasm” refers to an abnormal mass of tissue containing neoplastic cells. Neoplasms and tumors may be benign, premalignant, or malignant.

As used herein, the term “cancer” or “malignant neoplasm” refers to a cell that displays uncontrolled growth and division, invasion of adjacent tissues, and often metastasizes to other locations of the body.

As used herein, the term “antineoplastic” refers to a composition, such as a drug or biologic, that can inhibit or prevent cancer growth, invasion, and/or metastasis.

As used herein, the phrase “pharmaceutically acceptable” refers to compositions, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the phrase “pharmaceutically acceptable carrier” refers to pharmaceutically acceptable materials, compositions or vehicles, such as a liquid or solid filler, diluent, solvent or encapsulating material involved in carrying or transporting any subject composition, from one organ, or portion of the body, to another organ, or portion of the body. Each carrier must be “acceptable” in the sense of being compatible with the other ingredients of a subject composition and not injurious to the patient.

As used herein, the term “individual,” “subject,” and “patient” are used interchangeably to refer to any individual who is the target of administration or treatment. The subject can be a vertebrate, for example, a mammal. Thus, the subject can be a human or veterinary patient.

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

As used herein, the term “therapeutically effective amount” refers to an amount of the therapeutic agent that, when incorporated into and/or onto particles described herein, produces some desired effect at a reasonable benefit/risk ratio applicable to any medical treatment. The effective amount may vary depending on such factors as the disease or condition being treated, the particular targeted constructs being administered, the size of the subject, or the severity of the disease or condition. One of ordinary skill in the art may empirically determine the effective amount of a particular compound without necessitating undue experimentation. In some embodiments, the term “effective amount” refers to an amount of a therapeutic agent or prophylactic agent to reduce or diminish the symptoms of one or more diseases or disorders of the brain, such as reducing tumor size (e.g., tumor volume).

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%; in other forms the values may range in value either above or below the stated value in a range of approx. +/−5%; in other forms the values may range in value either above or below the stated value in a range of approx. +/−2%; in other forms the values may range in value either above or below the stated value in a range of approx. +/−1%. The preceding ranges are intended to be made clear by context, and no further limitation is implied.

As used herein, “optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. It should be understood that all of the individual values and sub-ranges of values contained within an explicitly disclosed range are also specifically contemplated and should be considered disclosed unless the context specifically indicates otherwise. Finally, it should be understood that all ranges refer both to the recited range as a range and as a collection of individual numbers from and including the first endpoint to and including the second endpoint. In the latter case, it should be understood that any of the individual numbers can be selected as one form of the quantity, value, or feature to which the range refers. In this way, a range describes a set of numbers or values from and including the first endpoint to and including the second endpoint from which a single member of the set (i.e. a single number) can be selected as the quantity, value, or feature to which the range refers. The foregoing applies regardless of whether in particular cases some or all of these embodiments are explicitly disclosed.

Every compound disclosed herein is intended to be and should be considered to be specifically disclosed herein. Further, every subgroup that can be identified within this disclosure is intended to be and should be considered to be specifically disclosed herein. As a result, it is specifically contemplated that any compound, or subgroup of compounds can be either specifically included for or excluded from use or included in or excluded from a list of compounds.

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular polypeptide is disclosed and discussed and a number of modifications that can be made to a number of polypeptides are discussed, specifically contemplated is each and every combination and permutation of polypeptides and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

### II. Compositions

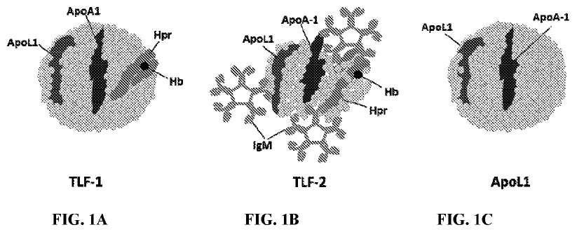

ApoL1 or ApoL1-containing complexes such as Trypanosome Lytic Factor (TLF: TLF-1 or TLF-2) (FIGS. 1A-1C)) are a minor sub-class of human High-density lipoproteins (HDLs). As used herein TLF encompasses both TLF-1 and TLF-2, though in each case may be more specifically substituted with TLF-1, TLF-2, or TLF-1 and TLF-2.

TLF is present in blood plasma at a level approximately 10 g/mL ((Samanovic, et al., PLoS Pathog., 5:e1000276 (2009), Bullard, et al., Virulence. 3:72-6 (2012)). In addition to ApoL1, ApoL1-containing complexes can include one or more of Haptoglobin related protein (Hpr), Apolipoprotein A1 (ApoA1), and IgM.

Hpr is important for TLF binding to a trypanosome-specific receptor present at ˜350 copies in the flagellar binding pocket of the parasite, but humans have no known receptor for this protein (Drain, et al., J Biol Chem., 276:30254-60 (2001)). A consensus amino acid sequence for Hpr is available at Unitprot Accession No. P00739·HPTR_HUMAN the entire contents of which is specifically incorporated by reference herein in its entirety, and provided as SEQ ID NO:49:

(SEQ ID NO: 49)
MSDLGAVISLLLWGRQLFALYSGNDVTDISDDRFPKPPEIANGYVEHLFRY
QCKNYYRLRTEGDGVYTLNDKKQWINKAVGDKLPECEAVCGKPKNPANPVQ
RILGGHLDAKGSFPWQAKMVSHHNLTTGATLINEQWLLTTAKNLFLNHSEN
ATAKDIAPTLTLYVGKKQLVEIEKVVLHPNYHQVDIGLIKLKQKVLVNERV
MPICLPSKNYAEVGRVGYVSGWGQSDNFKLTDHLKYVMLPVADQYDCITHY
EGSTCPKWKAPKSPVGVQPILNEHTFCVGMSKYQEDTCYGDAGSAFAVHDL
EEDTWYAAGILSFDKSCAVAEYGVYVKVTSIQHWVQKTIAEN.

ApoL1 is a member of the Bcl-2 family, a family in which its members play a crucial role in the regulation of programmed cell death (PCD) pathways. ApoL1 contains a BH3-domain pro-death region which has been identified as important for ACD as deletion of the domain in wild-type ApoL1 ablates its cytotoxicity (Wan, et al., J Biol Chem., 283:21540-9 (2008)). Four additional regions have been mapped on ApoL1: signal peptide (SP, aal-27), pore-forming domain (PFD, aa60-237), membrane association domain (MAD, aa238-303), and SRA-binding domain (aa339-398). The last three have been shown to be important for ApoL1 function and toxicity, although overexpression of any of the three domains did not lead to increased lethality (Lan, et al., Exp Mol Pathol. 99:139-44 (2015)). Of the six members within the ApoL family, ApoL1 is the only member that is secreted into the serum, with others functioning intracellularly (Vanhollebeke, et al., Cell Mol Life Sci CMLS., 63:1937-44 (2006)). While the exact role of ApoL1 has yet to be determined, two recently evolved variants, G1 and G2, have been correlated with an increased risk in chronic kidney diseases (Pant, et al., J Biol Chem., 297 (2021), Pays, et al., JAm Soc Nephrol., 31:2502-5 (2020)).

Despite the many unique disorders arising from ApoL1 associations (Pays, et al., Febs J., 288:360-81 (2021)), it has been identified as the primary component responsible for trypanosome killing (Vanhollebeke, et al., Mol Microbiol., 76:806-14 (2010)). Following uptake into acidic parasitic endosomes and lysosomal trafficking, ApoL1 undergoes pH mediated activation and can insert into lipid membranes forming closed-state pH-gated cation channels inducing irreversible osmotic damage to the parasite (Schaub, et al., J Biol Chem., 297 (2021), Harrington, et al., J Biol Chem., 284:13505-12 (2009)). A consensus amino acid sequence for ApoL1 is available at Uniprot Accession No. 014791 APOL1_HUMAN the entire contents of which is specifically incorporated by reference herein in its entirety, and provided as SEQ ID NO:50:

(SEQ ID NO: 50)
MEGAALLRVSVLCIWMSALFLGVGVRAEEAGARVQQNVPSGTDTGDPQSKP
LGDWAAGTMDPESSIFIEDAIKYFKEKVSTQNLLLLLTDNEAWNGEVAAAE
LPRNEADELRKALDNLARQMIMKDKNWHDKGQQYRNWELKEFPRLKSELED
NIRRLRALADGVQKVHKGTTIANVVSGSLSISSGILTLVGMGLAPFTEGGS
LVLLEPGMELGITAALTGITSSTMDYGKKWWTQAQAHDLVIKSLDKLKEVR
EFLGENISNFLSLAGNTYQLTRGIGKDIRALRRARANLOSVPHASASRPRV
TEPISAESGEQVERVNEPSILEMSRGVKLTDVAPVSFFLVLDVVYLVYESK
HLHEGAKSETAEELKKVAQELEEKLNILNNNYKILQADQEL.

Studies show that at concentrations exceeding physiological, TLF is able to elicit a similar cascade of lethal events in mammalian cells, although the direct mechanism has not yet been determined (Wan, et al., J Biol Chem., 283:21540-9 (2008)).

The results below show that ApoL1 and ApoL1-containing complexes such as TLF can be used to increase cell death of targeted cells, including but not limited to cancer cells. Thus disclosed are compositions for increasing ApoL1 in target cells, and methods of use thereof for inducing targeted cell death.

### A. Compositions for Targeting Endogenous ApoL1

Compositions for increasing the cellular internalization of endogenous ApoL1 are provided. Although not necessarily used exclusively for this purpose, the compositions can be used to recruit endogenous ApoL1 into cells. The compositions typically bind to ApoL1 and/or an ApoL1-containing complex such as those illustrated in FIGS. 1A-1C, and including but not limited to TLF. The compositions also typically bind to a cell specific marker, such as a cancer antigen, present on cells, thus facilitating targeting of the captured ApoL1 and/or ApoL1-containing complexes to the target cells. Cell specific markers and antigens are molecules that when targeted by a targeting moiety, e.g., an antibody or antigen binding fragment, can enhance delivery of the composition to the target cells. In some embodiments, the cell specific marker is elevated on the target cells relative to some or all other (non-target) cells, distinctive or unique to the target cells relative to some or all other (non-target cells), or a combination thereof.

The targeted ApoL1-containing complexes can be internalized by the target cells in an effective amount of increase cell death of the target cells. The compositions thus typically include a binding moiety that can specifically bind to ApoL1 or another component of a ApoL1-containing complex, and a targeting moiety can specifically recognize and bind to a target molecule specific for a cell type, a tissue type, or an organ (e.g., cell specific marker or antigen). The binding and/or target molecules can be a polypeptide, lipid, or glycolipid. The target molecule of the targeting moiety can be a receptor that is selectively expressed on a specific cell surface, a tissue or an organ. Cell specific markers can be for specific types of cells including, but not limited to stem cells, skin cells, blood cells, immune cells, muscle cells, nerve cells, cancer cells, virally infected cells, bacterial cells, fungal cells, organ specific cells, and other eukaryotic cells. The cell markers can be specific for endothelial, ectodermal, or mesenchymal cells. Representative cell specific markers include, but are not limited to, cancer specific markers. The cell markers can be any of the cell specific marker, including cancer and tumor antigens including, but not limited to, those provided elsewhere herein (see, e.g., below).

As discussed in more detail below, some embodiments, the targeting moiety targets non-mammalian cells, e.g., bacteria or fungi. Targets and targeting moieties for targeting such foreign cells are discussed in, for example, Mambro, et al., Sci Rep 11, 19500 (2021) doi.org/10.1038/s41598-021-98659-5, and Soniya, et al., 35(24):6636-6645 (2014), each of which is specifically incorporated by reference herein in its entirety, and which describe humanized monoclonal antibody specific for β-1,3 glucans, a component of several pathogenic fungi and a glyceryl-dilaurate lipid-moiety for targeting Plasmodium-infected red blood cells (iRBCs), respectively.

Typically the targeting moiety does not target a trypanosome specific surface antigen. In preferred embodiments, the composition is an antibody, preferably a bi- or multispecific antibody including antigen binding fragments for ApoL1 or an ApoL1-containing complex and a cell specific marker or antigen respectively.

1. Sequences for Antibodies that Bind ApoL1 or ApoL1-Containing Complexes

Provided herein are CDR and heavy and/or light chain sequences that bind to ApoL1-containing complexes such as TLF. In some embodiments, the antibodies bind to Hpr, e.g., Hpr having the sequence of SEQ ID NO:49. In some embodiments, the antibodies bind to ApoL1, e.g., ApoL1 having the sequence of SEQ ID NO:50. As discussed herein, antibodies that bind, preferably immunospecifically bind, Hpr or ApoL1, having one or more of the associated CDRs or variant thereof, and/or one or both the associated VH and VL sequences or variant thereof, in any and all antibody forms including, but not limited to, intact antibodies and antigen binding fragments, in monospecific, bispecific, and higher order multispecific formats, optionally as humanized or chimeric forms thereof are expressly provided. Thus, in some embodiments, the antibodies are, or include, fragments with antigen-binding capability (e.g., Fab′, F(ab′)2, Fab, Fv and rIgG, recombinant single chain Fv fragments (scFv) as well as divalent (di-scFv) and trivalent (tri-scFV) forms thereof.

The present disclosure additionally includes nucleic acid molecules (DNA or RNA) that encode any such antibodies, fusion proteins or fragments, as well as vector molecules (such as plasmids) that are capable of transmitting or of replication such nucleic acid molecules and expressing such antibodies, fusion proteins or fragments in a cell line, and the host cells transformed with such nucleic acids. The nucleic acids can be single-stranded, double-stranded, can contain both single-stranded and double-stranded portions.

a. Hpr Binding Sequences—Clone SFII 134.3

Clone SFII 134.3, a mouse IgG2a that binds to Hpr, was sequenced revealing the following variable domains and CDRs.

i. Heavy Chain

Variable Domain (VH)
(SEQ ID NO: 3)
QIQLVQSGPELKKPGETVKISCKASGYIFTNYGMNWVRQAPGKGLKWMGWI
NSYTGEATYTDDLKGRFAFSLESSASTAYLQINNLKNEDTATYFCAREGYG
DYGYSFDYWGQGTTLTVSS

TABLE 1
SFII 134.3 VH Complementary Determining Regions

| Scheme | CDR-H1 | CDR-H2 | CDR-H3 |
|---|---|---|---|
| Kabat | NYGMN (SEQ ID | WINSYTGEATYTDDLKG | EGYGDYGYSFDY (SEQ |
| | NO: 4) | (SEQ ID NO: 5) | ID NO: 6) |
| IMGT | GYIFTNYG | INSYTGEA (SEQ ID | AREGYGDYGYSFDY |
| | (SEQ ID NO: 7) | NO: 8) | (SEQ ID NO: 9) |
| Chothia | GYIFTNY (SEQ | NSYTGE (SEQ ID | EGYGDYGYSFDY (SEQ |
| | ID NO: 10) | NO: 11) | ID NO: 6) |

ii. Light Chain

Variable Domain (VL)
(SEQ ID NO: 14)
DIQMTQSPASLSASVGETVTITCRATKNIYTYLAWYQQKOGKSPQFLVYNA
KTLAEGVPSRFSGSGSGTQFSLKINSLQPEDFGNYYCQHHYGTPRTFGGGT
KLEIK

TABLE 2
SFII 134.3 VL Complementary Determining Regions

| Scheme | CDR-L1 | CDR-L2 | CDR-L3 |
|---|---|---|---|
| Kabat | RATKNIYTYLA | NAKTLAE (SEQ ID | QHHYGTPRT (SEQ |
| | (SEQ ID NO: 16) | NO: 17) | ID NO: 18) |
| IMGT | KNIYTY (SEQ ID | NAK | QHHYGTPRT (SEQ |
| | NO: 19) | | ID NO: 18) |
| Chothia | RATKNIYTYLA | NAKTLAE (SEQ ID | QHHYGTPRT (SEQ |
| | (SEQ ID NO: 16) | NO: 17) | ID NO: 18) |

In preferred embodiments, the antibodies and other molecules contain six CDRs. The CDRs can include at least one, two, three, four, five or six consensus CDRs of the CDRs of anti-Hpr antibody SFII 134.3, such as those provided herein.

For example, in some embodiments, the antibodies or other molecules contain at least one, two, three, four, five or six CDRs of the heavy and/or light chain variable domains of SEQ ID NO:3 and/or SEQ ID NO:14, respectively.

In some embodiments, the antibodies or other molecules include at least one, two, three, four, five, or six CDRs, optionally at least one CDR-H1, one CDR-H2, one CDR-H3, one CDR-L1, one CDR-L2, and one CDR-L3, of SFII 134.3 selected from:

SFII 134.3 CDR-H1:
(SEQ ID NO: 4)
NYGMN,
(SEQ ID NO: 7)
GYIFTNYG,
or
(SEQ ID NO: 10)
GYIFTNY,
or
a variant thereof with at least 70, 80, 90, or 95% sequence
identity thereto;
SFII 134.3 CDR-H2:
(SEQ ID NO: 5)
WINSYTGEATYTDDLKG,
(SEQ ID NO: 8)
INSYTGEA,
(SEQ ID NO: 11)
NSYTGE,
or
a variant thereof with at least 70, 80, 90, or 95% sequence
identity thereto;
SFII 134.3 CDR-H3:
(SEQ ID NO: 6)
EGYGDYGYSFDY,
(SEQ ID NO: 9)
AREGYGDYGYSFDY,
or
a variant thereof with at least 70, 80, 90, or 95% sequence
identity thereto;
SFII 134.3 CDR-L1:
(SEQ ID NO: 16)
RATKNIYTYLA,
(SEQ ID NO: 19)
KNIYTY,
or
a variant thereof with at least 70, 80, 90, or 95% sequence
identity thereto;
SFII 134.3 CDR-L2:,
(SEQ ID NO: 17)
NAKTLAE,
NAK,
or
a variant thereof with at least 70, 80, 90, or 95% sequence
identity thereto;
and
SFII 134.3 CDR-L3:
(SEQ ID NO: 18)
QHHYGTPRT,
or
a variant thereof with at least 70, 80, 90, or 95% sequence
identity thereto.

In some embodiments, the antibodies or other molecules includes the H1-H3 CDRs and L1-L3 CDRs of SFII 134.3, respectively, selected from:

Kabat:
(SEQ ID NO: 4)
NYGMN,
(SEQ ID NO: 5)
WINSYTGEATYTDDLKG,
(SEQ ID NO: 6)
EGYGDYGYSFDY,
(SEQ ID NO: 16)
RATKNIYTYLA,
(SEQ ID NO: 17)
NAKTLAE,
(SEQ ID NO: 18)
QHHYGTPRT,
or
variants thereof with at least 70, 80, 90, or 95% sequence
identity thereto;
IMGT:
(SEQ ID NO: 7)
GYIFTNYG,
(SEQ ID NO: 8)
INSYTGEA,
(SEQ ID NO: 9)
AREGYGDYGYSFDY,
(SEQ ID NO: 19)
KNIYTY,
NAK,
(SEQ ID NO: 18)
QHHYGTPRT,
or
variants thereof with at least 70, 80, 90, or 95% sequence
identity thereto;
or
Chothia:
(SEQ ID NO: 10)
GYIFTNY,
(SEQ ID NO: 11)
NSYTGE,
(SEQ ID NO: 6)
EGYGDYGYSFDY,
(SEQ ID NO: 16)
RATKNIYTYLA,
(SEQ ID NO: 17)
NAKTLAE,
(SEQ ID NO: 18)
QHHYGTPRT,
or
variants thereof with at least 70, 80, 90, or 95% sequence
identity thereto.

In some embodiments, the antibodies or other molecules include the heavy and/or light chain variable domains of SEQ ID NO:3 and/or SEQ ID NO:14, respectively, or a variant thereof with at least 70, 80, 90, or 95% sequence identity thereto.

b. Hpr Binding Sequences—Clone SFII 14.11

Clone SFII 14.11, a mouse IgG that binds to Hpr, was sequenced revealing the following variable domains and CDRs.

i. Heavy Chain

Variable Domain (VH)
(SEQ ID NO: 56)
QIQLVQSGPELKKPGETVKISCKASGFTFTDYSIHWVKQAPGKGLKWMGWK
HTESGESTYADDFKGRFVFSLETSASTAYLQINNLKNEDTSTYFCARGANY
GSLLDYWGQGTTLTVSS

TABLE 3
SFII 14.11 VH Complementary Determining Regions

| Scheme | CDR-H1 | CDR-H2 | CDR-H3 |
|---|---|---|---|
| Kabat | DYSIH (SEQ ID | WKHTESGESTYADDFKG | GANYGSLLDY (SEQ ID |
| | NO: 57) | (SEQ ID NO: 58) | NO: 59) |
| IMGT | GFTFTDYS | KHTESGES (SEQ ID | ARGANYGSLLDY (SEQ |
| | (SEQ ID | NO: 61) | ID NO: 62) |
| | NO: 60) | | |
| Chothia | GFTFTDY (SEQ | HTESGE (SEQ ID | GANYGSLLDY (SEQ ID |
| | ID NO: 63) | NO: 64) | NO: 59) |

ii. Light Chain

Variable Domain (VL)
(SEQ ID NO: 65)
DIVLTQSPASLAVSLGQRATISCRASKSVSTSGYSYMHWYQQKPGQSPKLL
IYLASNLESGVPARFSGSGSGTDFTLNIHPVEEEDAATYYCQHNRELPLTF
GAGTKLELKR

TABLE 4
SFII 14.11 VL Complementary Determining Regions

| Scheme | CDR-L1 | CDR-L2 | CDR-L3 |
|---|---|---|---|
| Kabat | RASKSVSTSGYSYMH | LASNLES (SEQ ID | QHNRELPLT (SEQ |
| | (SEQ ID NO: 66) | NO: 67) | ID NO: 68) |
| IMGT | KSVSTSGYSY (SEQ | LAS | QHNRELPLT (SEQ |
| | ID NO: 69) | | ID NO: 68) |
| Chothia | RASKSVSTSGYSYMH | LASNLES (SEQ ID | QHNRELPLT (SEQ |
| | (SEQ ID NO: 66) | NO: 67) | ID NO: 68) |

In preferred embodiments, the antibodies and other molecules contain six CDRs. The CDRs can include at least one, two, three, four, five or six consensus CDRs of the CDRs of anti-Hpr antibody 14.11, such as those provided herein.

For example, in some embodiments, the antibodies or other molecules contain at least one, two, three, four, five or six CDRs of the heavy and/or light chain variable domains of SEQ ID NO:56 and/or SEQ ID NO:65, respectively.

In some embodiments, the antibodies or other molecules include at least one, two, three, four, five, or six CDRs, optionally at least one CDR-H1, one CDR-H2, one CDR-H3, one CDR-L1, one CDR-L2, and one CDR-L3, of SFII 14.11 selected from:

SFII 14.11 CDR-H1:
(SEQ ID NO: 57)
DYSIH,
(SEQ ID NO: 58)
WKHTESGESTYADDFKG,
or
(SEQ ID NO: 59)
GANYGSLLDY,
or
a variant thereof with at least 70, 80, 90, or 95% sequence
identity thereto;
SFII 14.11 CDR-H2:
(SEQ ID NO: 60)
GFTFTDYS,
(SEQ ID NO: 61)
KHTESGES,
(SEQ ID NO: 62)
ARGANYGSLLDY,
or
a variant thereofwith at least 70, 80, 90, or 95% sequence
identity thereto;
SFII 14.11 CDR-H3:
(SEQ ID NO: 63)
GFTFTDY,
(SEQ ID NO: 64)
HTESGE,
or
(SEQ ID NO: 59)
GANYGSLLDY
or
a variant thereof with at least 70, 80, 90, or 95% sequence
identity thereto;
SFII 14.11 CDR-L1:
(SEQ ID NO: 66)
RASKSVSTSGYSYMH,
(SEQ ID NO: 69)
KSVSTSGYSY,,
or
a variant thereof with at least 70, 80, 90, or 95% sequence
identity thereto;
SFII 14.11 CDR-L2:
(SEQ ID NO: 67)
LASNLES,
LAS,
or
a variant thereof with at least 70, 80, 90, or 95% sequence
identity thereto;
and
SFII 14.11 CDR-L3:
(SEQ ID NO: 68)
QHNRELPLT,
or
a variant thereof with at least 70, 80, 90, or 95% sequence
identity thereto.

In some embodiments, the antibodies or other molecules includes the H1-H3 CDRs and L1-L3 CDRs of SFII 14.11, respectively, selected from:

Kabat:
(SEQ ID NO: 57)
DYSIH,
(SEQ ID NO: 58)
WKHTESGESTYADDEKG,
(SEQ ID NO: 59)
GANYGSLLDY,
(SEQ ID NO: 66)
RASKSVSTSGYSYMH,
(SEQ ID NO: 67)
LASNLES,
(SEQ ID NO: 68)
QHNRELPLT,
or
variants thereof with at least 70, 80, 90, or 95% sequence
identity thereto;
IMGT:
(SEQ ID NO: 60)
GFTFTDYS,
(SEQ ID NO: 61)
KHTESGES,
(SEQ ID NO: 62)
ARGANYGSLLDY,
(SEQ ID NO: 69)
KSVSTSGYSY,
LAS,
(SEQ ID NO: 68)
QHNRELPLT,
or
variants thereof with at least 70, 80, 90, or 95% sequence
identity thereto;
or
Chothia:
(SEQ ID NO: 63)
GFTFTDY,
(SEQ ID NO: 64)
HTESGE,
(SEQ ID NO: 59)
GANYGSLLDY,
(SEQ ID NO: 66)
RASKSVSTSGYSYMH,
(SEQ ID NO: 67)
LASNLES,
(SEQ ID NO: 68)
QHNRELPLT,
or
variants thereof with at least 70, 80, 90, or 95% sequence
identity thereto.

In some embodiments, the antibodies or other molecules include the heavy and/or light chain variable domains of SEQ ID NO:56 and/or SEQ ID NO:65, respectively, or a variant thereof with at least 70, 80, 90, or 95% sequence identity thereto.

c. ApoL1 Binding Sequences

Clone SFIII 13.11, a mouse IgG1 that binds ApoL1, was sequenced revealing the following variable domains and CDRs.

i. Heavy Chain

Variable Domain (VH)
(SEQ ID NO: 24)
EVQLVESGGGLVKPGGSLKLSCAASGFTFSTYAMSWVRQSPEKRLEWVAEI
SNGGLYTYYPDTVTGRFTISRDNVKNILYLEMSSLRSEDTAIYYCIRENRN
WYFDLWGAGTTVTVSS

TABLE 5
SFIII 13.11 VH Complementary Determining Regions

| Scheme | CDR-H1 | CDR-H2 | CDR-H3 |
|---|---|---|---|
| Kabat | TYAMS (SEQ | EISNGGLYTYYPDTVTG | ENRNWYFDL |
| | ID NO: 25) | (SEQ ID NO: 26) | (SEQ ID NO: 27) |
| IMGT | GFTESTYA | ISNGGLYT (SEQ ID | IRENRNWYFDL |
| | (SEQ ID | NO: 29) | (SEQ ID NO: 30) |
| | NO: 28) | | |
| Chothia | GFTFSTY | SNGGLY (SEQ ID | ENRNWYFDL |
| | (SEQ ID | NO: 32) | (SEQ ID NO: 27) |
| | NO: 31) | | |

ii. Light Chain

Variable Domain (VL)
(SEQ ID NO: 36)
DVLMTQTPLSLPVSLGDQASISCRSSQSIVNSNGNTYLEWYLQKPGQSPKL
LIYKVSNRFSGVPDRFSGSGSGTDFTLKISRVEAEDLGVYYCFQGSHVPLT
FGAGTKLELK;
or
(SEQ ID NO: 77)
DVLMTQTPLSLPVSLGDQASISCRSSQSIVNSNGNTYLEWYLQKPGQSPKL
LIYKVSNRFSGVPDRFSGSGSGTDFTLKISRVEAEDLGVYYCFQGSHVPLT
FGAGTKLEIK

TABLE 6
SFIII 13.11 VL Complementary Determining Regions

| Scheme | CDR-L1 | CDR-L2 | CDR-L3 |
|---|---|---|---|
| Kabat | RSSQSIVNSNGNTYLE | KVSNRFS (SEQ | FQGSHVPLT (SEQ ID |
| | (SEQ ID NO: 37) | ID NO: 38) | NO: 39) |
| IMGT | QSIVNSNGNTY (SEQ | KVS | FQGSHVPLT (SEQ ID |
| | ID NO: 40) | | NO: 39) |
| Chothia | RSSQSIVNSNGNTYLE | KVSNRFS (SEQ | FQGSHVPLT (SEQ ID |
| | (SEQ ID NO: 37) | ID NO: 38) | NO: 39) |

In preferred embodiments, the antibodies and other molecules contain six CDRs. The CDRs can include at least one, two, three, four, five or six consensus CDR of the CDRs of anti-ApoL1 antibody SFIII 13.11.

For example, in some embodiments, the antibodies or other molecules contain at least one, two, three, four, five or six CDRs of the heavy and/or light chain variable domains of SEQ ID NO:24 and/or SEQ ID NO:36 or SEQ ID NO:77, respectively.

In some embodiments, the antibodies or other molecules include at least one, two, three, four, five, or six CDRs, optionally at least one CDR-H1, one CDR-H2, one CDR-H3, one CDR-L1, one CDR-L2, and one CDR-L3, of SFIII 13.11 selected from:

SFIII 13.11 CDR-H1:
(SEQ ID NO: 25)
TYAMS,
(SEQ ID NO: 28)
GFTFSTYA,
(SEQ ID NO: 31)
GFTFSTY,
or
a variant thereof with at least 70, 80, 90, or 95% sequence
identity thereto;
SFIII 13.11 CDR-H2:
(SEQ ID NO: 26)
EISNGGLYTYYPDTVTG,
(SEQ ID NO: 29)
ISNGGLYT,
(SEQ ID NO: 32)
SNGGLY,
or
a variant thereof with at least 70, 80, 90, or 95% sequence
identity thereto;
SFIII 13.11 CDR-H3:
(SEQ ID NO: 27)
ENRNWYFDL,
(SEQ ID NO: 30)
IRENRNWYFDL,
or
a variant thereof with at least 70, 80, 90, or 95% sequence
identity thereto;
SFIII 13.11 CDR-L1:
(SEQ ID NO: 37)
RSSQSIVNSNGNTYLE,
(SEQ ID NO: 40)
QSIVNSNGNTY,
or
a variant thereof with at least 70, 80, 90, or 95% sequence
identity thereto;
SFIII 13.11 CDR-L2:
(SEQ ID NO: 38)
KVSNRFS,
KVS,
or
a variant thereof with at least 70, 80, 90, or 95% sequence
identity thereto;
and
SFIII 13.11 CDR-L3:,
(SEQ ID NO: 39)
FQGSHVPLT,
or
a variant thereof with at least 70, 80, 90, or 95% sequence
identity thereto.

In some embodiments, the antibodies or other molecules includes the H1-H3 CDR and L1-L3 CDRs of SFIII 13.11, respectively, selected from:

Kabat:
(SEQ ID NO: 25)
TYAMS,
(SEQ ID NO: 26)
EISNGGLYTYYPDTVTG,
(SEQ ID NO: 27)
ENRNWYFDL,
(SEQ ID NO: 37)
RSSQSIVNSNGNTYLE,
(SEQ ID NO: 38)
KVSNRFS,
(SEQ ID NO: 39)
FQGSHVPLT,
or
variants thereof with at least 70, 80, 90, or 95% sequence
identity thereto;
IMGT:
(SEQ ID NO: 28)
GFTFSTYA,
(SEQ ID NO: 29)
ISNGGLYT,
(SEQ ID NO: 30)
IRENRNWYFDL,
(SEQ ID NO: 40)
QSIVNSNGNTY,
KVS,
(SEQ ID NO: 39)
FQGSHVPLT,
or
variants thereof with at least 70, 80, 90, or 95% sequence
identity thereto;
or
Chothia:
(SEQ ID NO: 31)
GFTFSTY,
(SEQ ID NO: 32)
SNGGLY,
(SEQ ID NO: 27)
ENRNWYFDL,
(SEQ ID NO: 37)
RSSQSIVNSNGNTYLE,
(SEQ ID NO: 38)
KVSNRFS,
(SEQ ID NO: 39)
FQGSHVPLT,
or
variants thereof with at least 70, 80, 90, or 95% sequence
identity thereto.

In some embodiments, the antibodies or other molecules include the heavy and/or light chain variable domains of SEQ ID NO:24 and/or SEQ ID NO:36 or SEQ ID NO:77, respectively, or a variant thereof with at least 70, 80, 90, or 95% sequence identity thereto.

### 2. Chimeric and Humanized Antibodies

The disclosure particularly concerns chimeric and humanized antibodies. Constant regions need not be present, but if they are, are typically substantially identical to human immunoglobulin constant regions, i.e., at least about 85-90%, preferably about 95% or more identical. Hence, all parts of a humanized immunoglobulin, except possibly the CDR's, are substantially identical to corresponding parts of natural human immunoglobulin sequences. A humanized antibody is an antibody having a humanized light chain and a humanized heavy chain immunoglobulin. For example, a humanized antibody would not encompass a typical chimeric antibody, because, e.g., the entire variable region of a chimeric antibody is non-human. One says that the donor antibody has been “humanized,” by the process of “humanization,” because the resultant humanized antibody is expected to bind to the same antigen as the donor antibody that provides the CDR's.

For the most part, humanized antibodies are human immunoglobulins (recipient antibody) in which hypervariable region residues of the recipient are replaced by hypervariable region residues from a non-human species (donor antibody) such as mouse, rat, rabbit or a non-human primate having the desired specificity, affinity, and capacity. In some instances, Framework Region (FR) residues of the human immunoglobulin are replaced by corresponding non-human residues. Furthermore, humanized antibodies can include residues which are not found in the recipient antibody or in the donor antibody. These modifications are made to further refine antibody performance. In general, the humanized antibody will include substantially all of at least one, and typically two, variable domains, in which all or substantially all of the hypervariable regions correspond to those of a non-human immunoglobulin and all or substantially all of the FRs are those of a human immunoglobulin sequence. The humanized antibody optionally also will include at least a portion of an immunoglobulin constant region (Fc), typically that of a human immunoglobulin that immunospecifically binds to an FcγRIIB polypeptide, that has been altered by the introduction of amino acid residue substitutions, deletions or additions (i.e., mutations). See also, e.g., European Patent Nos. EP 239,400, EP 592,106, and EP 519,596; International Publication Nos. WO 91/09967 and WO 93/17105; U.S. Pat. Nos. 5,225,539, 5,530,101, 5,565,332, 5,585,089, 5,766,886, and 6,407,213; and Padlan, 1991, Molecular Immunology 28(4/5):489-498; Studnicka et al., 1994, Protein Engineering 7(6):805-814; Roguska et al., 1994, PNAS 91:969-973; Tan et al., 2002, J Immunol. 169:1119-1125; Caldas et al., 2000, Protein Eng. 13:353-360; Morea et al., 2000, Methods 20:267-79; Baca et al., 1997, J. Biol. Chem. 272:10678-10684; Roguska et al., 1996, Protein Eng. 9:895-904; Couto et al., 1995, Cancer Res. 55 (23 Supp):5973s-5977s; Couto et al., 1995, Cancer Res. 55:1717-22; Sandhu, 1994, Gene 150:409-10; Pedersen et al., 1994, J. Mol. Biol. 235:959-973; Jones et al., 1986, Nature 321:522-525; Reichmann et al., 1988, Nature 332:323-329; and Presta, 1992, Curr. Op. Struct. Biol. 2:593-596).

DNA sequences coding for preferred human acceptor framework sequences include but are not limited to FR segments from the human germline VH segment VH1-18 and JH6 and the human germline VL segment VK-A26 and JK4. In a specific embodiment, one or more of the CDRs are inserted within framework regions using routine recombinant DNA techniques. The framework regions can be naturally occurring or consensus framework regions, and preferably human framework regions (see, e.g., Chothia et al., 1998, “Structural Determinants In The Sequences Of Immunoglobulin Variable Domain,” J Mol. Biol. 278: 457-479 for a listing of human framework regions).

A humanized or chimeric antibodies can include substantially all of at least one, and typically two, variable domains in which all or substantially all of the CDR regions correspond to those of a non-human immunoglobulin (i.e., donor antibody) and all or substantially all of the framework regions are those of a human immunoglobulin consensus sequence. Preferably, the antibody also includes at least a portion of an immunoglobulin constant region (Fc), typically that of a human immunoglobulin. The constant domains of the antibodies can be selected with respect to the proposed function of the antibody, in particular the effector function which can be required. In some embodiments, the constant domains of the antibodies are (or include) human IgA, IgD, IgE, IgG or IgM domains. In a specific embodiment, human IgG constant domains, especially of the IgG1 and IgG3 isotypes are used, when the humanized antibodies are intended for therapeutic uses and antibody effector functions such as antibody-dependent cell-mediated cytotoxicity (ADCC) and complement-dependent cytotoxicity (CDC) activity are needed. In alternative embodiments, IgG2 and IgG4 isotypes are used when the antibody is intended for therapeutic purposes and antibody effector function is not required. The disclosure encompasses Fc constant domains including one or more amino acid modifications which alter antibody effector functions such as those disclosed in U.S. Patent Application Publication Nos. 2005/0037000 and 2005/0064514.

In some embodiments, the antibody contains both the light chain as well as at least the variable domain of a heavy chain. In other embodiments, the antibody can further include one or more of the CH1, hinge, CH2, CH3, and CH4 regions of the heavy chain. The antibody can be selected from any class of immunoglobulins, including IgM, IgG, IgD, IgA and IgE, and any isotype, including IgG1, IgG2, IgG3 and IgG4. In some embodiments, the constant domain is a complement fixing constant domain where it is desired that the antibody exhibit cytotoxic activity, and the class is typically IgG1. In other embodiments, where such cytotoxic activity is not desirable, the constant domain can be of the IgG2 class. The antibody can include sequences from more than one class or isotype, and selecting particular constant domains to optimize desired effector functions is within the ordinary skill in the art. In some embodiments, the antibody is not a mouse IgG1 or a mouse IgG2a.

The framework and CDR regions of a humanized antibody need not correspond precisely to the parental sequences, e.g., the donor CDR or the consensus framework can be mutagenized by substitution, insertion or deletion of at least one residue so that the CDR or framework residue at that site does not correspond to either the consensus or the donor antibody. Such mutations, however, are preferably not extensive. Usually, at least 75% of the humanized antibody residues will correspond to those of the parental framework region (FR) and CDR sequences, more often 90%, and most preferably greater than 95%. Humanized antibodies can be produced using variety of techniques known in the art, including, but not limited to, CDR-grafting (European Patent No. EP 239,400; International Publication No. WO 91/09967; and U.S. Pat. Nos. 5,225,539, 5,530,101, and 5,585,089), veneering or resurfacing (European Patent Nos. EP 592,106 and EP 519,596; Padlan, 1991, Molecular Immunology 28(4/5):489-498; Studnicka et al., 1994, Protein Engineering 7(6):805-814; and Roguska et al., 1994, Proc. Natl. Acad. Sci. 91:969-973), chain shuffling (U.S. Pat. No. 5,565,332), and techniques disclosed in, e.g., U.S. Pat. Nos. 6,407,213, 5,766,886, 5,585,089, International Publication No. WO 9317105, Tan et al., 2002, J. Immunol. 169:1119-25, Caldas et al., 2000, Protein Eng. 13:353-60, Morea et al., 2000, Methods 20:267-79, Baca et al., 1997, J. Biol. Chem. 272:10678-84, Roguska et al., 1996, Protein Eng. 9:895-904, Couto et al., 1995, Cancer Res. 55 (23 Supp):5973s-5977s, Couto et al., 1995, Cancer Res. 55:1717-22, Sandhu, 1994, Gene 150:409-10, Pedersen et al., 1994, J. Mol. Biol. 235:959-73, Jones et al., 1986, Nature 321:522-525, Riechmann et al., 1988, Nature 332:323, and Presta, 1992, Curr. Op. Struct. Biol. 2:593-596. Often, framework residues in the framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; U.S. Publication Nos. 2004/0049014 and 2003/0229208; U.S. Pat. Nos. 6,350,861; 6,180,370; 5,693,762; 5,693,761; 5,585,089; and 5,530,101 and Riechmann et al., 1988, Nature 332:323).

### 3. Bispecific and Multispecific Antibodies

The antibodies used in the methods of the present disclosure can be monospecific. Antibodies monospecific for ApoL1 or ApoL1-containing complexes can have a targeting moiety conjugated or otherwise linked thereto. In some embodiments, the targeting moiety is an antibody or antigen binding fragment thereof. Thus, also of interest are bispecific antibodies, trispecific antibodies or antibodies of greater multispecificity that exhibit specificity to different targets in addition to ApoL1 or Hpr. For example, such antibodies can bind to both ApoL1 or Hpr and to an antigen that is important for targeting the antibody to a particular cell type or tissue (for example, to an antigen associated with a cancer antigen of a tumor being treated).

### a. Exemplary Structures of Bi- and Multispecific Molecules

In some embodiments, the antibodies are heterodimeric bi- and tri-(or more) specific Ig antibodies and Fc fusion proteins. Exemplary structures include, but are not limited to, IgG, IgM, mono-, di-, tri-, or more scFv-Fcs. For example, bispecific, trispecific, and multispecific formats include, but are not limited to, bispecific and trispecific IgG, IgG-scFv, IgG-dAb, scFv-Fc-scFv, knob-in-hole (KIH)-IgG, κλ-body, KIH0Fc-Fab/scFv, tandem scFv, KIH trispecific, bispecific Fc fusion (N- or C-terminal, with or without KIH).

In embodiments, multispecific antibody molecules can include more than one antigen-binding site, where different sites are specific for different antigens. In embodiments, multispecific antibody molecules can bind more than one (e.g., two or more) epitopes on the same antigen. In embodiments, multispecific antibody molecules include an antigen-binding site specific for a target cell (e.g., cancer cell) and a different antigen-binding site specific for ApoL1-containing complexes such as TLF (e.g., Hpr or ApoL1). In some embodiments, the multispecific antibody molecule is a bispecific antibody molecule. Bispecific antibody molecules can be classified into five different structural groups: (i) bispecific immunoglobulin G (BsIgG); (ii) IgG appended with an additional antigen-binding moiety; (iii) bispecific antibody fragments; (iv) bispecific fusion proteins; and (v) bispecific antibody conjugates.

BsIgG is a format that is monovalent for each antigen. Exemplary BsIgG formats include but are not limited to crossMab, DAF (two-in-one), DAF (four-in-one), DutaMab, DT-IgG, knobs-in-holes common LC, knobs-in-holes assembly, charge pair, Fab-arm exchange, SEEDbody, triomab, LUZ-Y, Fcab, kappa-lamda-body, orthogonal Fab. See Spiess et al. Mol. Immunol. 67(2015):95-106. Exemplary BsIgGs include catumaxomab (Fresenius Biotech, Trion Pharma, Neopharm), which contains an anti-CD3 arm and an anti-EpCAM arm; and ertumaxomab (Neovii Biotech, Fresenius Biotech), which targets CD3 and HER2.

In some embodiments, BsIgG includes heavy chains that are engineered for heterodimerization. For example, heavy chains can be engineered for heterodimerization using a “knobs-into-holes” strategy, a SEED platform, a common heavy chain (e.g., in Kk-bodies), and use of heterodimeric Fe regions. See Spiess et al., Mol. Immunol. 67(2015):95-106. Strategies that have been used to avoid heavy chain pairing of homodimers in BsIgG include knobs-in-holes, duobody, azymetric, charge pair, HA-TF, SEEDbody, and differential protein A affinity. See Id. BsIgG can be produced by separate expression of the component antibodies in different host cells and subsequent purification/assembly into a BsIgG. BsIgG can also be produced by expression of the component antibodies in a single host cell. BsIgG can be purified using affinity chromatography, e.g., using protein A and sequential pH elution.

IgG appended with an additional antigen-binding moiety is another format of bispecific antibody molecules. For example, monospecific IgG can be engineered to have bispecificity by appending an additional antigen-binding unit onto the monospecific IgG, e.g., at the N- or C-terminus of either the heavy or light chain. Exemplary additional antigen-binding units include single domain antibodies (e.g., variable heavy chain or variable light chain), engineered protein scaffolds, and paired antibody variable regions (e.g., single chain variable fragments or variable fragments). See Id. Examples of appended IgG formats include dual variable domain (DVD) IgG (DVD-Ig), IgG(H)-scFv, scFv-(H)IgG, IgG(L)-scFv, scFv-(L)IgG, IgG(L,H)-Fv, IgG(H)-V, V(H)—IgG, IgG(L)-V, V(L)-IgG, KIH IgG-scFab, 2scFv-IgG, IgG-2scFv, scFv4-Ig, zybody, and DVI-IgG (four-in-one). See Spiess et al. Mol. Immunol. 67(2015):95-106. An example of an IgG-scFv is MM-141 (Merrimack Pharmaceuticals), which binds IGF-1R and HER3. Examples of DVD-Ig include ABT-981 (AbbVie), which binds IL-1alpha and IL-1beta; and ABT-122 (AbbVie), which binds TNF and IL-17A.

Bispecific antibody fragments (BsAb) are a format of bispecific antibody molecules that lack some or all of the antibody constant domains. For example, some BsAb lack an Fc region. In some embodiments, bispecific antibody fragments include heavy and light chain regions that are connected by a peptide linker that permits efficient expression of the BsAb in a single host cell. Exemplary bispecific antibody fragments include but are not limited to nanobody, nanobody-HAS, BiTE, Diabody, DART, TandAb, scDiabody, scDiabody-CH3, Diabody-CH3, triple body, miniantibody, minibody, TriBi minibody, scFv-CH3 KIH, Fab-scFv, scFv-CH-CL-scFv, F(ab′)2, F(ab′)2-scFv2, scFv-KIH, Fab-scFv-Fc, tetravalent HCAb, scDiabody-Fc, Diabody-Fc, tandem scFv-Fc, and intrabody. See Id. For example, the BiTE format includes tandem scFvs, where the component scFvs bind to CD3 on T cells and a surface antigen on cancer cells.

Bispecific fusion proteins include antibody fragments linked to other proteins, e.g., to add additional specificity and/or functionality. An example of a bispecific fusion protein is an immTAC, which includes an anti-CD3 scFv linked to an affinity-matured T-cell receptor that recognizes HLA-presented peptides. In embodiments, the dock-and-lock (DNL) method can be used to generate bispecific antibody molecules with higher valency. Also, fusions to albumin binding proteins or human serum albumin can be extend the serum half-life of antibody fragments. See Id.

In embodiments, chemical conjugation, e.g., chemical conjugation of antibodies and/or antibody fragments, can be used to create BsAb molecules. See Id An exemplary bispecific antibody conjugate includes the CovX-body format, in which a low molecular weight drug is conjugated site-specifically to a single reactive lysine in each Fab arm or an antibody or fragment thereof. In embodiments, the conjugation improves the serum half-life of the low molecular weight drug. An exemplary CovX-body is CVX-241 (NCT01004822), which includes an antibody conjugated to two short peptides inhibiting either VEGF or Ang2. See Id

In some embodiments the multispecific molecule further includes a heavy chain constant region (e.g., an Fc region) chosen from the heavy chain constant regions of IgG1, IgG2, and IgG4, more particularly, the heavy chain constant region of human IgG1, IgG2 or IgG4. In some embodiments, the heavy chain constant region (e.g., an Fc region) is linked to, e.g., covalently linked to, one or both of the ApoL1-containing complex-binding antibody molecule and the second antibody molecule.

In some embodiments, the heavy chain constant region (e.g., an Fc region) is altered, e.g., mutated, to increase or decrease one or more of Fc receptor binding, antibody glycosylation, the number of cysteine residues, effector cell function, or complement function. In some embodiments, an interface of a first and second heavy chain constant regions (e.g., Fc region) is altered, e.g., mutated, to increase or decrease dimerization, e.g., relative to a non-engineered interface. In some embodiments, the dimerization of the heavy chain constant region (e.g., Fc region) is enhanced by providing an Fc interface of a first and a second Fc region with one or more of a paired cavity-protuberance (“knob-in-a hole”), an electrostatic interaction, or a strand-exchange, such that a greater ratio of heteromultimer:homomultimer forms, e.g., relative to a non-engineered interface. In some embodiments, the heavy chain constant region (e.g., Fc region) includes an amino acid substitution at a position chosen from one or more of 347, 349, 350, 351, 366, 368, 370, 392, 394, 395, 397, 398, 399, 405, 407, or 409, e.g., of the Fc region of human IgG1, numbered based on the Eu numbering system. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md., 1991.

In some embodiments, the heavy chain constant region (e.g., Fc region) includes an amino acid substitution chosen from: T366S, L368A, or Y407V (e.g., corresponding to a cavity or hole), or T366W (e.g., corresponding to a protuberance or knob), or a combination thereof, numbered based on the Eu numbering system.

In some embodiments, the heavy chain constant region (e.g., an Fc region) includes one or more mutations that increase or decrease one or more of: Fc receptor binding, antibody glycosylation, the number of cysteine residues, effector cell function, or complement function, relative to a naturally-existing heavy chain constant region.

In some embodiments, the ApoL1-containing complex-binding molecule includes a first heavy chain constant region (e.g., a first Fc region) and the second antibody molecule includes a second heavy chain constant region (e.g., a second Fc region), wherein the first heavy chain constant region includes one or more mutations that increase heterodimerization of the first heavy chain constant region and the second heavy chain constant region, relative to a naturally-existing heavy chain constant region, and/or wherein the second heavy chain constant region includes one or more mutations that increase heterodimerization of the second heavy chain constant region and the first heavy chain constant region, relative to a naturally-existing heavy chain constant region. In some embodiments, the first and the second heavy chain constant regions (e.g., first and second Fc regions) include one or more of a paired cavity-protuberance (“knob-in-a hole”), an electrostatic interaction, or a strand-exchange, such that a greater ratio of heteromultimer:homomultimer forms, e.g., relative to naturally-existing heavy chain constant regions.

In some embodiments, the first and/or second heavy chain constant region (e.g., a first and/or second Fc region, e.g., a first and/or second IgG1 Fc region) includes an amino acid substitution at a position chosen from one or more of 347, 349, 350, 351, 366, 368, 370, 392, 394, 395, 397, 398, 399, 405, 407, or 409, numbered based on the Eu numbering system. In some embodiments, the first and/or second heavy chain constant region (e.g., a first and/or second Fc region, e.g., a first and/or second IgG1 Fc region) includes an amino acid substitution chosen from: T366S, L368A, Y407V, or Y349C (e.g., corresponding to a cavity or hole), or T366W or S354C (e.g., corresponding to a protuberance or knob), or a combination thereof, numbered based on the Eu numbering system.

In some embodiments, the multispecific molecule further includes a linker, e.g., a linker between one or more of: the ApoL1-containing complex-binding molecule and the second antibody molecule, the ApoL1-containing complex-binding antibody molecule and the heavy chain constant region (e.g., the Fc region), or the second antibody molecule and the heavy chain constant region. In some embodiments, the linker is chosen from: a cleavable linker, a non-cleavable linker, a peptide linker, a flexible linker, a rigid linker, a helical linker, or a non-helical linker. In some embodiments, the linker is a peptide linker. In some embodiments, the peptide linker includes Gly and Ser.

### b. Cancer Antigens

In some embodiments, the disclosed antibodies that bind to ApoL1-containing complexes such as TLF are bi- or other multispecific molecules that also bind a cancer antigen.

In some embodiments, the cancer antigen is an antigen of a blood cancer such as multiple myeloma, leukemia (e.g., chronic lymphocytic leukemia, acute myeloid leukemia, acute lymphoblastic leukemia), non-Hodgkin lymphoma, Hodgkin lymphoma, myelodysplastic syndromes (MDS), myeloproliferative neoplasms (MPNs) (or a subcategory thereof, e.g., essential thrombocythemia (ET), myelofibrosis (MF) and polycythemia vera (PV), amyloidosis, Waldenstrom macroglobulinemia, or aplastic anemia. In other embodiments, the cancer antigen is an antigen of a solid tumor.

Common blood cancer antigens include, but are not limited to, BCMA, PD-L1, CTLA-4, CD38, CD319/SLAMF-7, TNFRSF17/BCMA, SYND1/CD138, CD229, CD47, CD123/IL3-RA, CD19, CD20, CD22, FcRH5, GPRC5D, and CLL-1. Thus, in some embodiments, the bi- or multispecific antibody includes an antigen binding fragment that specifically binds to BCMA, PD-L1, CTLA-4, CD38, CD319/SLAMF-7, TNFRSF17/BCMA, SYND1/CD138, CD229, CD47, CD123/IL3-RA, CD19, CD20, CD22, FcRH5, GPRC5D, or CLL-1.

Cancer and tumor antigens of known structure and having a known or described function, include the following cell surface receptors: HERI (GenBank Accession NO: U48722), HER2 (Yoshino, et al., J. Immunol., 152:2393 (1994); Disis, et al., Canc. Res., 54:16 (1994); GenBank Acc. Nos. X03363 and M17730), HER3 (GenBank Acc. Nos. U29339 and M34309), HER4 (Plowman, et al., Nature, 366:473 (1993); GenBank Acc. Nos. L07868 and T64105), epidermal growth factor receptor (EGFR) (GenBank Acc. Nos. U48722, and KO3193), vascular endothelial cell growth factor (GenBank NO: M32977), vascular endothelial cell growth factor receptor (GenBank Acc. Nos. AF022375, 1680143, U48801 and X62568), insulin-like growth factor-I (GenBank Acc. Nos. X00173, X56774, X56773, X06043, European Patent No. GB 2241703), insulin-like growth factor-II (GenBank Acc. Nos. X03562, X00910, M17863 and M17862), transferrin receptor (Trowbridge and Omary, Proc. Nat. Acad. USA, 78:3039 (1981); GenBank Acc. Nos. X01060 and M1,1507), estrogen receptor (GenBank Acc. Nos. M38651, X03635, X99101, U47678 and M12674), progesterone receptor (GenBank Acc. Nos. X51730, X69068 and M15716), follicle stimulating hormone receptor (FSH-R) (GenBank Acc. Nos. Z34260 and M65085), retinoic acid receptor (GenBank Acc. Nos. L12060, M60909, X77664, X57280, X07282 and X06538), MUC-1 (Barnes, et al., Proc. Nat. Acad. Sci. USA, 86:7159 (1989); GenBank Acc. Nos. M65132 and M64928) NY-ESO-1 (GenBank Acc. Nos. AJ003149 and U87459), NA 17-A (PCT Publication NO: WO 96/40039), Melan-A/MART-1 (Kawakami, et al., Proc. Nat. Acad. Sci. USA, 91:3515 (1994); GenBank Acc. Nos. U06654 and U06452), tyrosinase (Topalian, et al., Proc. Nat. Acad. Sci. USA, 91:9461 (1994); GenBank Acc. NO: M26729; Weber, et al., J. Clin. Invest, 102:1258 (1998)), Gp-100 (Kawakami, et al., Proc. Nat. Acad. Sci. USA, 91:3515 (1994); GenBank Acc. NO: S73003, Adema, et al., J Biol. Chem., 269:20126 (1994)), MAGE (van den Bruggen, et al., Science, 254:1643 (1991)); GenBank Acc. Nos. U93163, AF064589, U66083, D32077, D32076, D32075, U10694, U10693, U10691, U10690, U10689, U10688, U10687, U10686, U10685, L18877, U10340, U10339, L18920, U03735 and M77481), BAGE (GenBank Acc. NO: U19180; U.S. Pat. Nos. 5,683,886 and 5,571,711), GAGE (GenBank Acc. Nos. AF055475, AF055474, AF055473, U19147, U19146, U19145, U19144, U19143 and U19142), any of the CTA class of receptors including in particular HOM-MEL-40 antigen encoded by the SSX2 gene (GenBank Acc. Nos. X86175, U90842, U90841 and X86174), carcinoembryonic antigen (CEA, Gold and Freedman, J. Exp. Med, 121:439 (1985); GenBank Acc. Nos. M59710, M59255 and M29540), PyLT (GenBank Acc. Nos. J02289 and J02038); p97 (melanotransferrin) (Brown, et al., J Immunol., 127:539-46 (1981); Rose, et al., Proc. Natd. Acad. Sci. USA, 83:1261-61 (1986), neuroblastoma antigen PTK7, and B7-DC (PD-L2).

Additional tumor associated antigens include prostate surface antigen (PSA) (U.S. Pat. Nos. 6,677,157; 6,673,545); β-human chorionic gonadotropin β-HCG) (McManus, et al., Cancer Res., 36:3476-81 (1976); Yoshimura, et al., Cancer, 73:2745-52 (1994); Yamaguchi, et al., Br. J Cancer, 60:382-84 (1989): Alfthan, et al., Cancer Res., 52:4628-33 (1992)); glycosyltransferase β-1,4-N-acetylgalactosaminyltransferases (GalNAc) (Hoon, et al., Int. J. Cancer, 43:857-62 (1989); Ando, et al., Int. J Cancer, 40:12-17 (1987); Tsuchida, et al., J Natl. Cancer, 78:45-54 (1987); Tsuchida, et al., J Natl. Cancer, 78:55-60 (1987)); NUC18 (Lehmann, et al., Proc. Natl. Acad. Sci. USA, 86:9891-95 (1989); Lehmann, et al., Cancer Res., 47:841-45 (1987)); melanoma antigen gp75 (Vijayasardahi, et al., J. Exp. Med., 171:1375-80 (1990); GenBank Accession NO: X51455); human cytokeratin 8; high molecular weight melanoma antigen (Natali, et al., Cancer, 59:55-63 (1987); keratin 19 (Datta, et al., J. Clin. Oncol., 12:475-82 (1994)).

Tumor antigens of interest include antigens regarded in the art as “cancer/testis” (CT) antigens that are immunogenic in subjects having a malignant condition (Scanlan, et al., Cancer Immun., 4:1 (2004)). CT antigens include at least 19 different families of antigens that contain one or more members and that are capable of inducing an immune response, including, but not limited to, MAGEA (CT1); BAGE (CT2); MAGEB (CT3); GAGE (CT4); SSX (CT5); NY-ESO-1 (CT6); MAGEC (CT7); SYCP1 (C8); SPANXB1 (CT11.2); NA88 (CT18); CTAGE (CT21); SPA17 (CT22); OY-TES-1 (CT23); CAGE (CT26); HOM-TES-85 (CT28); HCA661 (CT30); NY-SAR-35 (CT38); FATE (CT43); and TPTE (CT44).

Additional tumor antigens that can be targeted, including a tumor-associated or tumor-specific antigen, include. but are not limited to, alpha-actinin-4, Bcr-Abl fusion protein, Casp-8, beta-catenin, cdc27, cdk4, cdkn2a, coa-1, dek-can fusion protein, EF2, ETV6-AML1 fusion protein, LDLR-fucosyltransferaseAS fusion protein, HLA-A2, HLA-A11, hsp70-2, KIAAO205, Mart2, Mum-1, 2, and 3, neo-PAP, myosin class I, OS-9, pml-RARα fusion protein, PTPRK, K-ras, N-ras, Triosephosphate isomeras, Bage-1, Gage 3,4,5,6,7, GnTV, Herv-K-mel, Lage-1, Mage-A1,2,3,4,6,10,12, Mage-C2, NA-88, NY-Eso-1/Lage-2, SP17, SSX-2, and TRP2-Int2, MelanA (MART-I), gp100 (Pmel 17), tyrosinase, TRP-1, TRP-2, MAGE-1, MAGE-3, BAGE, GAGE-1, GAGE-2, p15(58), CEA, RAGE, NY-ESO (LAGE), SCP-1, Hom/Mel-40, PRAME, p53, H-Ras, HER-2/neu, BCR-ABL, E2A-PRL, H4-RET, IGH-IGK MYL-RAR, Epstein Barr virus antigens, EBNA, human papillomavirus (HPV) antigens E6 and E7, TSP-180, MAGE-4, MAGE-5, MAGE-6, p185erbB2, p180erbB-3, c-met, nm-23H1, PSA, TAG-72-4, CA 19-9, CA 72-4, CAM 17.1, NuMa, K-ras, β-Catenin, CDK4, Mum-1, p16, TAGE, PSMA, PSCA, CT7, telomerase, 43-9F, 5T4, 791Tgp72, α-fetoprotein, 13HCG, BCA225, BTAA, CA 125, CA 15-3 (CA 27.29BCAA), CA 195, CA 242, CA-50, CAM43, CD68KP1, CO-029, FGF-5, G250, Ga733 (EpCAM), HTgp-175, M344, MA-50, MG7-Ag, MOV18, NB\70K, NY-CO-1, RCAS1, SDCCAG16, TA-90 (Mac-2 binding protein\cyclophilin C-associated protein), TAAL6, TAG72, TLP, and TPS. Other tumor-associated and tumor-specific antigens are known to those of skill in the art and are suitable for targeting by the disclosed fusion proteins.

In particular embodiments, the tumor antigen is a pancreatic cancer antigen, optionally selected from Claudin 18.2, MUC1, Mesothelin (MSLN), and Myoferlin (MYOF).

MYOF can be used to target pancreatic ductal adenocarcinoma (PDAC) (Gupta, et al., Nat Cell Biol 23, 232-242 (2021), non-small cell lung cancer (Song, et al., Oncol Lett. 11(2): 998-1006 (2016)), doi:10.3892/ol.2015.3988, and breast cancer (Zhang, et al., Nat Commun. 9(1):3726 (2018) doi: 10.1038/s41467-018-06179-0).

Mesothelin (MSLN) is additionally found in ovarian cancer, lung adenocarcinoma, malignant mesothelioma, biliary cancer, gastric cancer, and pediatric acute myeloid leukemia (Hassan and Ho, et al., Eur J Cancer., 44(1): 46-53 (2008), Hassan, et al., J Clin Oncol. 34(34):4171-4179. doi: 10.1200/JCO.2016.68.3672 (2016)).

Thus, in some embodiments, the bi- or multispecific antibody includes an antigen binding fragment that specifically binds to Claudin 18.2, MUC1, Mesothelin (MSLN), and Myoferlin (MYOF).

In other embodiments, the tumor antigen is a melanoma cancer antigen, optionally PMEL17. Thus, in some embodiments, the bi- or multispecific antibody includes an antigen binding fragment that specifically binds to PMEL17.

Typically, the cell marker or antigen is not a trypanosome specific surface antigen.

In preferred embodiments, the foregoing antigens are targeted with an antibody that binds thereto. Thus, for all of the provided tumor antigens, also provided are antibodies and antigen binding fragments that specifically bind thereof. In other embodiments, the targeting moiety is not an antibody, and can be, for example, another polypeptide, a carbohydrate, lipid, etc. as discussed elsewhere herein.

### 4. Derivatives and Conjugates

The disclosure particularly contemplates the production and use of derivatives of any of the above-described antibodies and their antigen-binding fragments. The term derivative encompasses an antibody or antigen-binding fragment thereof that immunospecifically binds to an antigen but which includes, one, two, three, four, five or more amino acid substitutions, additions, deletions or modifications relative to a “parental” (or wild-type) molecule (also referred to as variants). Such amino acid substitutions or additions can introduce naturally occurring (i.e., DNA-encoded) or non-naturally occurring amino acid residues.

The term derivative also encompasses, for example, chimeric or humanized variants of any of the disclosed antibodies, as well as variants having altered CH1, hinge, CH2, CH3 or CH4 regions, so as to form, for example antibodies, etc., having variant Fc regions that exhibit enhanced or impaired effector or binding characteristics.

The term derivative additionally encompasses non-amino acid modifications, for example, amino acids that may be glycosylated (e.g., have altered mannose, 2-N-acetylglucosamine. galactose, fucose, glucose, sialic acid, 5-N-acetylneuraminic acid, 5-glycolneuraminic acid, etc. content), acetylated, pegylated, phosphorylated, amidated, derivatized by known protecting/blocking groups, proteolytic cleavage, linked to a cellular ligand or other protein, etc. In some embodiments, the altered carbohydrate modifications modulate one or more of the following: solubilization of the antibody, facilitation of subcellular transport and secretion of the antibody, promotion of antibody assembly, conformational integrity, and antibody-mediated effector function. In a specific embodiment the altered carbohydrate modifications enhance antibody mediated effector function relative to the antibody lacking the carbohydrate modification. Carbohydrate modifications that lead to altered antibody mediated effector function are well known in the art (for example, see Shields, R. L. et al. (2002) “Lack Of Fucose On Human IgG N-Linked Oligosaccharide Improves Binding To Human Fcgamma RIII And Antibody-Dependent Cellular Toxicity.,” J. Biol. Chem. 277(30): 26733-26740; Davies J. et al. (2001) “Expression Of GnTIII In A Recombinant Anti-CD20 CHO Production Cell Line: Expression Of Antibodies With Altered Glycoforms Leads To An Increase In ADCC Through Higher Affinity For FC Gamma RIII,” Biotechnology & Bioengineering 74(4): 288-294). Methods of altering carbohydrate contents are known to those skilled in the art, see, e.g., Wallick, S.C. et al. (1988) “Glycosylation Of A VH Residue Of A Monoclonal Antibody Against Alpha (1 - - - 6) Dextran Increases Its Affinity For Antigen,” J. Exp. Med. 168(3): 1099-1109; Tao, M. H. et al. (1989) “Studies Of Aglycosylated Chimeric Mouse-Human IgG. Role Of Carbohydrate In The Structure And Effector Functions Mediated By The Human IgG Constant Region,” J. Immunol. 143(8): 2595-2601; Routledge, E. G. et al. (1995) “The Effect Of Aglycosylation On The Immunogenicity Of A Humanized Therapeutic CD3 Monoclonal Antibody,” Transplantation 60(8):847-53; Elliott, S. et al. (2003) “Enhancement Of Therapeutic Protein In Vivo Activities Through Glycoengineering,” Nature Biotechnol. 21:414-21; Shields, R. L. et al. (2002) “Lack Of Fucose On Human IgG N-Linked Oligosaccharide Improves Binding To Human Fcgamma RIII And Antibody-Dependent Cellular Toxicity.,” J. Biol. Chem. 277(30): 26733-26740).

In some embodiments, a humanized antibody is a derivative. Such a humanized antibody includes amino acid residue substitutions, deletions or additions in one or more non-human CDRs. The humanized antibody derivative can have substantially the same binding, better binding, or worse binding when compared to a non-derivative humanized antibody. In specific embodiments, one, two, three, four, or five amino acid residues of the CDRs have been substituted, deleted or added (i.e., mutated).

A derivative antibody or antibody fragment can be modified by chemical modifications using techniques known to those of skill in the art, including, but not limited to, specific chemical cleavage, acetylation, formulation, metabolic synthesis of tunicamycin, etc. In one embodiment, an antibody derivative will possess a similar or identical function as the parental antibody. In another embodiment, an antibody derivative will exhibit an altered activity relative to the parental antibody. For example, a derivative antibody (or fragment thereof) can bind to its epitope more tightly or be more resistant to proteolysis than the parental antibody.

Derivatized antibodies can be used to alter the half-lives (e.g., serum half-lives) of parental antibodies in a mammal, preferably a human. Preferably such alteration will result in a half-life of greater than 15 days, preferably greater than 20 days, greater than 25 days, greater than 30 days, greater than 35 days, greater than 40 days, greater than 45 days, greater than 2 months, greater than 3 months, greater than 4 months, or greater than 5 months. The increased half-lives of the humanized antibodies of the present disclosure or fragments thereof in a mammal, preferably a human, results in a higher serum titer of said antibodies or antibody fragments in the mammal, and thus, reduces the frequency of the administration of said antibodies or antibody fragments and/or reduces the concentration of said antibodies or antibody fragments to be administered. Antibodies or fragments thereof having increased in vivo half-lives can be generated by techniques known to those of skill in the art. For example, antibodies or fragments thereof with increased in vivo half-lives can be generated by modifying (e.g., substituting, deleting or adding) amino acid residues identified as involved in the interaction between the Fc domain and the FcRn receptor. The humanized antibodies can be engineered to increase biological half-lives (see, e.g. U.S. Pat. No. 6,277,375). For example, humanized antibodies can be engineered in the Fc-hinge domain to have increased in vivo or serum half-lives.

Antibodies or fragments thereof with increased in vivo half-lives can be generated by attaching to said antibodies or antibody fragments polymer molecules such as high molecular weight polyethyleneglycol (PEG). PEG can be attached to said antibodies or antibody fragments with or without a multifunctional linker either through site-specific conjugation of the PEG to the N- or C- terminus of said antibodies or antibody fragments or via epsilon-amino groups present on lysine residues. Linear or branched polymer derivatization that results in minimal loss of biological activity will be used. The degree of conjugation will be closely monitored by SDS-PAGE and mass spectrometry to ensure proper conjugation of PEG molecules to the antibodies. Unreacted PEG can be separated from antibody-PEG conjugates by, e.g., size exclusion or ion-exchange chromatography.

The antibodies can also be modified by the methods and coupling agents described by Davis et al. (See U.S. Pat. No. 4,179,337) in order to provide compositions that can be injected into the mammalian circulatory system with substantially no immunogenic response.

One embodiment encompasses modification of framework residues of the humanized ApoL1 or Hpr antibodies. Framework residues in the framework regions can be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See, e.g., U.S. Pat. No. 5,585,089; and Riechmann, L. et al. (1988) “Reshaping Human Antibodies For Therapy,” Nature 332:323-327).

Yet another embodiment encompasses anti-ApoL1 and anti-Hpr antibodies (and more preferably, humanized antibodies) and antigen-binding fragments thereof that are recombinantly fused or chemically conjugated (including both covalently and non-covalently conjugations) to a heterologous molecule (i.e., an unrelated molecule). The fusion does not necessarily need to be direct, but can occur through linker sequences.

In one embodiment such heterologous molecules are polypeptides having at least 10, at least 20, at least 30, at least 40, at least 50, at least 60, at least 70, at least 80, at least 90 or at least 100 amino acids. Such heterologous molecules can alternatively be enzymes, hormones, cell surface receptors, drug moieties, such as: toxins (such as abrin, ricin A, Pseudomonas exotoxin (i.e., PE-40), diphtheria toxin, ricin, gelonin, or pokeweed antiviral protein), proteins (such as tumor necrosis factor, interferon (e.g., α-interferon, β-interferon), nerve growth factor, platelet derived growth factor, tissue plasminogen activator, or an apoptotic agent (e.g., tumor necrosis factor-α, tumor necrosis factor-β)), biological response modifiers (such as, for example, a lymphokine (e.g., interleukin-1 (“IL-1”), interleukin-2 (“IL-2”), interleukin-6 (“IL-6”)), granulocyte macrophage colony stimulating factor (“GM-CSF”), granulocyte colony stimulating factor (“G-CSF”), or macrophage colony stimulating factor, (“M-CSF”)), or growth factors (e.g., growth hormone (“GH”))), cytotoxins (e.g., a cytostatic or cytocidal agent, such as paclitaxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof), antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, BiCNU® (carmustine; BSNU) and lomustine (CCNU), cyclothosphamide, busulfan, dibromomannitol, streptozotocin, mitomycin C, and cisdichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), or anti-mitotic agents (e.g., vincristine and vinblastine).

Techniques for conjugating such therapeutic moieties to antibodies are well known; see, e.g., Amon et al., “Monoclonal Antibodies For Immunotargeting Of Drugs In Cancer Therapy”, in MONOCLONAL ANTIBODIES AND CANCER THERAPY, Reisfeld et al. (eds.), 1985, pp. 243-56, Alan R. Liss, Inc.); Hellstrom et al., Antibodies For Drug Delivery”, in CONTROLLED DRUG DELIVERY (2nd Ed.), Robinson et al. (eds.), 1987, pp. 623-53, Marcel Dekker, Inc.); Thorpe, “Antibody Carriers Of Cytotoxic Agents In Cancer Therapy: A Review”, in MONOCLONAL ANTIBODIES '84: BIOLOGICAL AND CLINICAL APPLICATIONS, Pinchera et al. (eds.), 1985, pp. 475-506); “Analysis, Results, And Future Prospective Of The Therapeutic Use Of Radiolabeled Antibody In Cancer Therapy”, in MONOCLONAL ANTIBODIES FOR CANCER DETECTION AND THERAPY, Baldwin et al. (eds.), 1985, pp. 303-16, Academic Press; and Thorpe et al. (1982) “The Preparation And Cytotoxic Properties Of Antibody-Toxin Conjugates,” Immunol. Rev. 62:119-158.

In one embodiment, the ApoL1 and Hpr antibodies or fusion molecules include an Fc portion. The Fc portion of such molecules can be varied by isotype or subclass, can be a chimeric or hybrid, and/or can be modified, for example to improve effector functions, control of half-life, tissue accessibility, augment biophysical characteristics such as stability, and improve efficiency of production (and less costly). Many modifications useful in construction of disclosed fusion proteins and methods for making them are known in the art, see for example Mueller, J. P. et al. (1997) “Humanized Porcine VCAM-Specific Monoclonal Antibodies With Chimeric IgG2/G4 Constant Regions Block Human Leukocyte Binding To Porcine Endothelial Cells,” Mol. Inmmun. 34(6):441-452, Swaim, P.G. (2008) “Considerations For The Development Of Therapeutic Monoclonal Antibodies,” Curr. Opin. Immun. 20:493-499 (2008), and Presta, L.G. (2008) “Molecular Engineering And Design Of Therapeutic Antibodies,” Curr. Opin. Immun. 20:460-470. In some embodiments the Fc region is the native IgG1, IgG2, or IgG4 Fc region. In some embodiments the Fc region is a hybrid, for example a chimeric having IgG2/IgG4 Fc constant regions. Modifications to the Fc region include, but are not limited to, IgG4 modified to prevent binding to Fc gamma receptors and complement, IgG1 modified to improve binding to one or more Fc gamma receptors, IgG1 modified to minimize effector function (amino acid changes), IgG1 with altered/no glycan (typically by changing expression host), and IgG1 with altered pH-dependent binding to FcRn, and IgG4 with serine at amino acid resident #228 in the hinge region changed to proline (S228P) to enhance stability. The Fc region can include the entire hinge region, or less than the entire hinge region.

The therapeutic outcome in patients treated with rituximab (a chimeric mouse/human IgG1 monoclonal antibody against CD20) for non-Hodgkin's lymphoma or Waldenstrom's macroglobulinemia correlated with the individual's expression of allelic variants of Fcγ receptors with distinct intrinsic affinities for the Fc domain of human IgG1. In particular, patients with high affinity alleles of the low affinity activating Fc receptor CD16A (FcγRIIIA) showed higher response rates and, in the cases of non-Hodgkin's lymphoma, improved progression-free survival. In another embodiment, the Fc domain can contain one or more amino acid insertions, deletions or substitutions that reduce binding to the low affinity inhibitory Fc receptor CD32B (FcγRIIB) and retain wild-type levels of binding to or enhance binding to the low affinity activating Fc receptor CD16A (FcγRIIIA).

Another embodiment includes IgG2-4 hybrids and IgG4 mutants that have reduce binding to FcR which increase their half-life. Representative IG2-4 hybrids and IgG4 mutants are described in Angal, S. et al. (1993) “A Single Amino Acid Substitution Abolishes The Heterogeneity Of Chimeric Mouse Human (Igg4) Antibody,” Molec. Immunol. 30(1):105-108; Mueller, J. P. et al. (1997) “Humanized Porcine VCAM-Specific Monoclonal Antibodies With Chimeric Igg2/G4 Constant Regions Block Human Leukocyte Binding To Porcine Endothelial Cells,” Mol. Immun. 34(6):441-452; and U.S. Pat. No. 6,982,323. In some embodiments the IgG₁ and/or IgG₂ domain is deleted for example, Angal, s. et al. describe IgG₁ and IgG₂ having serine 241 replaced with a proline.

Substitutions, additions or deletions in the derivatized antibodies can be in the Fc region of the antibody and can thereby serve to modify the binding affinity of the antibody to one or more FcγR. Methods for modifying antibodies with modified binding to one or more FcγR are known in the art, see, e.g., PCT Publication Nos. WO 04/029207, WO 04/029092, WO 04/028564, WO 99/58572, WO 99/51642, WO 98/23289, WO 89/07142, WO 88/07089, and U.S. Pat. Nos. 5,843,597 and 5,642,821. In one particular embodiment, the modification of the Fc region results in an antibody with an altered antibody-mediated effector function, an altered binding to other Fc receptors (e.g., Fc activation receptors), an altered antibody-dependent cell-mediated cytotoxicity (ADCC) activity, an altered C1q binding activity, an altered complement-dependent cytotoxicity activity (CDC), a phagocytic activity, or any combination thereof.

In some embodiments, the disclosure encompasses antibodies whose Fc region will have been modified so that the molecule will exhibit altered Fc receptor (FcR) binding activity, for example to exhibit decreased activity toward activating receptors such as FcγRIIA or FcγRIIIA, or increased activity toward inhibitory receptors such as FcγRIIB. Preferably, such antibodies will exhibit decreased antibody-dependent cell-mediated cytotoxicity (ADCC) or complement dependent cytotoxicity (CDC) activities (relative to a wild-type Fc receptor).

Modifications that affect Fc-mediated effector function are well known in the art (see U.S. Pat. No. 6,194,551, and WO 00/42072; Stavenhagen, J. B. et al. (2007) “Fc Optimization Of Therapeutic Antibodies Enhances Their Ability To Kill Tumor Cells In Vitro And Controls Tumor Expansion In Vivo Via Low-Affinity Activating Fcgamma Receptors,” Cancer Res. 57(18):8882-8890; Shields, R. L. et al. (2001) “High Resolution Mapping of the Binding Site on Human IgG1for FcγRI, FcγRII, FcγRIII. and FcRn and Design ofIgGJ Variants with Improved Binding to the FcγR,” J. Biol. Chem. 276(9):6591-6604). Exemplary variants of human IgG1 Fc domains with reduced binding to FcγRIIA or FcγRIIIA, but unchanged or enhanced binding to FcγRIIB, include S239A, H268A, S267G, E269A, E293A, E293D, Y296F, R301A, V303A, A327G, K322A, E333A, K334A, K338A, A339A, D376A.

In some embodiments, the disclosure encompasses antibodies whose Fc region will have been deleted (for example, a Fab or F(ab)₂, etc.).

Any of the molecules of the present disclosure can be fused to marker sequences, such as a peptide, to facilitate purification. In preferred embodiments, the marker amino acid sequence is a hexa-histidine peptide, the hemagglutinin “HA” tag, which corresponds to an epitope derived from the influenza hemagglutinin protein (Wilson, I. A. et al. (1984) “The Structure Of An Antigenic Determinant In A Protein,” Cell, 37:767-778) and the “flag” tag (Knappik, A. et al. (1994) “An Improved Affinity Tag Based On The FLAG Peptide For The Detection And Purification Of Recombinant Antibody Fragments,” Biotechniques 17(4):754-761).

The present disclosure also encompasses antibodies or their antigen-binding fragments that are conjugated to a diagnostic or therapeutic agent or any other molecule for which serum half-life is desired to be increased. The antibodies can be used diagnostically (in vivo, in situ or in vitro) to, for example, monitor the development or progression of a disease, disorder or infection as part of a clinical testing procedure to, e.g., determine the efficacy of a given treatment regimen. Detection can be facilitated by coupling the antibody to a detectable substance. Examples of detectable substances include various enzymes, prosthetic groups, fluorescent materials, luminescent materials, bioluminescent materials, radioactive materials, positron emitting metals, and nonradioactive paramagnetic metal ions. The detectable substance can be coupled or conjugated either directly to the antibody or indirectly, through an intermediate (such as, for example, a linker known in the art) using techniques known in the art. See, for example, U.S. Pat. No. 4,741,900 for metal ions which can be conjugated to antibodies for use as diagnostics according to the present disclosure. Such diagnosis and detection can be accomplished by coupling the antibody to detectable substances including, but not limited to, various enzymes, enzymes including, but not limited to, horseradish peroxidase, alkaline phosphatase, beta-galactosidase, or acetylcholinesterase; prosthetic group complexes such as, but not limited to, streptavidin/biotin and avidin/biotin; fluorescent materials such as, but not limited to, umbelliferone, fluorescein, fluorescein isothiocyanate, rhodamine, dichlorotriazinylamine fluorescein, dansyl chloride or phycoerythrin; luminescent material such as, but not limited to, luminol; bioluminescent materials such as, but not limited to, luciferase, luciferin, and aequorin; radioactive material such as, but not limited to, bismuth (²¹³Bi), carbon (¹⁴C), chromium (⁵¹Cr), cobalt (⁵⁷Co), fluorine (¹⁸F), gadolinium (¹⁵³Gd, ¹⁵⁹Gd), gallium (⁶⁸Ga, ⁶⁷Ga), germanium (⁶⁸Ge), holmium (¹⁶⁶Ho), indium (¹¹⁵In, ¹¹³In, ¹¹²In, ¹¹¹In), iodine (¹³¹I ¹²⁵I, ¹²³I, ¹²¹I), lanthanum (¹⁴⁰La), lutetium (¹⁷⁷Lu), manganese (⁵⁴Mn), molybdenum (⁹⁹Mo), palladium (¹⁰³Pd), phosphorous (³²P), praseodymium (¹²Pr), promethium (¹⁴⁹Pm), rhenium (¹⁸⁶Re, ¹⁸⁸Re), rhodium (¹⁰⁵Rh) ruthenium (⁹⁷Ru), samarium (¹⁵³Sm), scandium (⁴⁷Sc), selenium (⁷⁵Se), strontium (⁸⁵Sr), sulfur (³⁵S), technetium (⁹⁹Tc), thallium (²⁰¹Ti), tin (¹¹³Sn, ¹¹⁷Sn), tritium (³H), xenon (¹³³Xe), ytterbium (¹⁶⁹Yb, ¹⁷⁵Yb), yttrium (⁹⁰Y), zinc (⁶⁵Zn); positron emitting metals using various positron emission tomographies, and nonradioactive paramagnetic metal ions.

The molecules of the present disclosure can be conjugated to a second antibody to form an antibody heteroconjugate as described by Segal in U.S. Pat. No. 4,676,980. Such heteroconjugate antibodies may additionally bind to haptens (such as fluorescein, etc.), or to cellular markers, or to cytokines, or chemokines (e.g., CCL21), etc.

The molecules of the present disclosed can be attached to solid supports, which are particularly useful for immunoassays or purification of the target antigen or of other molecules that are capable of binding to target antigen that has been immobilized to the support via binding to an antibody or antigen-binding fragment of the present disclosure. Such solid supports include, but are not limited to, glass, cellulose, polyacrylamide, nylon, polystyrene, polyvinyl chloride or polypropylene.

### 5. Methods of Making Antibodies and Antigen Binding Fragments

The disclosed antibodies can be produced by any method known in the art useful for the production of polypeptides, e.g., in vitro synthesis, recombinant DNA production, and the like. Preferably, the antibodies are produced by recombinant DNA technology. The antibodies can be produced using recombinant immunoglobulin expression technology. The recombinant production of immunoglobulin molecules, including humanized antibodies are described in U.S. Pat. No. 4,816,397 (Boss et al.), U.S. Pat. Nos. 6,331,415 and 4,816,567 (both to Cabilly et al.), U.K. patent GB 2,188,638 (Winter et al.), and U.K. patent GB 2,209,757. Techniques for the recombinant expression of immunoglobulins. including humanized immunoglobulins, can also be found, in Goeddel et al., Gene Expression Technology Methods in Enzymology Vol. 185 Academic Press (1991), and Borreback, Antibody Engineering, W. H. Freeman (1992). Additional information concerning the generation, design and expression of recombinant antibodies can be found in Mayforth, Designing Antibodies, Academic Press, San Diego (1993).

An exemplary process for the production of the recombinant chimeric antibodies can include the following: a) constructing, by conventional molecular biology methods, an expression vector that encodes and expresses an antibody heavy chain in which the CDRs and variable region of a murine anti-ApoL1 or anti-Hpr monoclonal antibody are fused to an Fc region derived from a human immunoglobulin, thereby producing a vector for the expression of a chimeric antibody heavy chain; b) constructing, by conventional molecular biology methods, an expression vector that encodes and expresses an antibody light chain of the murine anti-ApoL1 or anti-Hpr monoclonal antibody, thereby producing a vector for the expression of chimeric antibody light chain; c) transferring the expression vectors to a host cell by conventional molecular biology methods to produce a transfected host cell for the expression of chimeric antibodies; and d) culturing the transfected cell by conventional cell culture techniques so as to produce chimeric antibodies.

An exemplary process for the production of the recombinant humanized antibodies can include the following: a) constructing, by conventional molecular biology methods, an expression vector that encodes and expresses an anti-ApoL1 or anti-Hpr heavy chain in which the CDRs and a minimal portion of the variable region framework that are required to retain donor antibody binding specificity are derived from a non-human immunoglobulin, such as a murine anti-ApoL1 or anti-Hpr monoclonal antibody, and the remainder of the antibody is derived from a human immunoglobulin, thereby producing a vector for the expression of a humanized antibody heavy chain; b) constructing, by conventional molecular biology methods, an expression vector that encodes and expresses an antibody light chain in which the CDRs and a minimal portion of the variable region framework that are required to retain donor antibody binding specificity are derived from anon-human immunoglobulin, such as a murine anti-ApoL1 or anti-Hpr monoclonal antibody, and the remainder of the antibody is derived from a human immunoglobulin, thereby producing a vector for the expression of humanized antibody light chain; c) transferring the expression vectors to a host cell by conventional molecular biology methods to produce a transfected host cell for the expression of humanized antibodies; and d) culturing the transfected cell by conventional cell culture techniques so as to produce humanized antibodies.

With respect to either exemplary method, host cells can be co-transfected with such expression vectors, which can contain different selectable markers but, with the exception of the heavy and light chain coding sequences, are preferably identical. This procedure provides for equal expression of heavy and light chain polypeptides. Alternatively, a single vector can be used which encodes both heavy and light chain polypeptides. The coding sequences for the heavy and light chains can include cDNA or genomic DNA or both. The host cell used to express the recombinant antibody can be either a bacterial cell such as Escherichia coli, or more preferably a eukaryotic cell (e.g., a Chinese hamster ovary (CHO) cell or a HEK-293 cell). The choice of expression vector is dependent upon the choice of host cell, and can be selected so as to have the desired expression and regulatory characteristics in the selected host cell. Other cell lines that can be used include, but are not limited to, CHO-K1, NSO, and PER.C6 (Crucell, Leiden, Netherlands).

Any of the above-described antibodies can be used to generate anti-idiotype antibodies using techniques well known to those skilled in the art (see, e.g., Greenspan, N. S. et al. (1989) “Idiotypes: Structure And Immunogenicity,” FASEB J. 7:437-444; and Nisinoff, A. (1991) “Idiotypes: Concepts And Applications,” J. Immunol. 147(8):2429-2438).

The binding properties of any of the above antibodies can, if desired, be further improved by screening for variants that exhibit such desired characteristics. For example, such antibodies can be generated using various phage display methods known in the art. In phage display methods, functional antibody domains are displayed on the surface of phage particles which carry the polynucleotide sequences encoding them. In a particular embodiment, such phage can be utilized to display antigen binding domains, such as Fab and Fv or disulfide-bond stabilized Fv, expressed from a repertoire or combinatorial antibody library (e.g., human or murine). Phage expressing an antigen binding domain that binds the antigen of interest can be selected or identified with antigen, e.g., using labeled antigen or antigen bound or captured to a solid surface or bead. Phage used in these methods are typically filamentous phage, including fd and M13. The antigen binding domains are expressed as a recombinantly fused protein to either the phage gene III or gene VIII protein. Examples of phage display methods that can be used to make the immunoglobulins, or fragments thereof, of the present disclosure include those disclosed in Brinkman, U. et al. (1995) “Phage Display Of Disulfide-Stabilized Fv Fragments,” J. Immunol. Methods, 182:41-50, 1995; Ames, R. S. et al. (1995) “Conversion Of Murine Fabs Isolated From A Combinatorial Phage Display Library To Full Length Immunoglobulins,” J. Immunol. Methods, 184:177-186; Kettleborough, C.A. et al. (1994) “Isolation Of Tumor Cell-Specific Single-Chain Fv From Immunized Mice Using Phage-Antibody Libraries And The Re-Construction Of Whole Antibodies From These Antibody Fragments,” Eur. J. Immunol., 24:952-958, 1994; Persic, L. et al. (1997) “An Integrated Vector System For The Eukaryotic Expression Of Antibodies Or Their Fragments After Selection From Phage Display Libraries,” Gene, 187:9-18; Burton, D. R. et al. (1994) “Human Antibodies From Combinatorial Libraries,” Adv. Immunol. 57:191-280; PCT Publications WO 92/001047; WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108.

As described in the above references, after phage selection, the antibody coding regions from the phage can be isolated and used to generate whole antibodies, including humanized antibodies, or any other desired fragments, and expressed in any desired host, including mammalian cells, insect cells, plant cells, yeast, and bacteria, e.g., as described in detail below. For example, techniques to recombinantly produce Fab, Fab′ and F(ab′)₂ fragments can also be employed using methods known in the art (such as those disclosed in PCT Publication WO 92/22324; Mullinax, R. L. et al. (1992) “Expression Of A Heterodimeric Fab Antibody Protein In One Cloning Step,” BioTechniques, 12(6):864-869; and Sawai et al. (1995) “Direct Production Of The Fab Fragment Derived From The Sperm Immobilizing Antibody Using Polymerase Chain Reaction And cDNA Expression Vectors,” Am. J. Reprod. Immunol. 34:26-34; and Better, M. et al. (1988) “Escherichia coli Secretion Of An Active Chimeric Antibody Fragment,” Science 240:1041-1043). Examples of techniques which can be used to produce single-chain Fvs and antibodies include those described in U.S. Pat. Nos. 4,946,778 and 5,258,498; Huston, J. S. et al. (1991) “Protein Engineering Of Single-Chain Fv Analogs And Fusion Proteins,” Methods in Enzymology 203:46-88; Shu, L. et al., “Secretion Of A Single-Gene-Encoded Immunoglobulin From Myeloma Cells,” Proc. Natl. Acad. Sci. (USA) 90:7995-7999; and Skerra. A. et al. (1988) “Assembly Of A Functional Immunoglobulin Fv Fragment In Escherichia coli,” Science 240:1038-1040.

Phage display technology can be used to increase the affinity of an antibody for ApoL1 or Hpr. This technique would be useful in obtaining high affinity antibodies that could be used in the disclosed methods. This technology, referred to as affinity maturation, employs mutagenesis or CDR walking and re-selection using such receptors or ligands (or their extracellular domains) or an antigenic fragment thereof to identify antibodies that bind with higher affinity to the antigen when compared with the initial or parental antibody (See, e.g., Glaser, S. M. et al. (1992) “Antibody Engineering By Codon-Based Mutagenesis In A Filamentous Phage Vector System,” J. Immunol. 149:3903-3913). Mutagenizing entire codons rather than single nucleotides results in a semi-randomized repertoire of amino acid mutations. Libraries can be constructed including of a pool of variant clones each of which differs by a single amino acid alteration in a single CDR and which contain variants representing each possible amino acid substitution for each CDR residue. Mutants with increased binding affinity for the antigen can be screened by contacting the immobilized mutants with labeled antigen. Any screening method known in the art can be used to identify mutant antibodies with increased avidity to the antigen (e.g., ELISA) (see, e.g., Wu, H. et al. (1998) “Stepwise In Vitro Affinity Maturation Of Vitaxin, An Alphav Beta3-Specific Humanized Mab,” Proc. Natl. Acad. Sci. (USA) 95(11):6037-6042; Yelton, D.E. et al. (1995) “Affinity Maturation Of The BR96Anti-Carcinoma Antibody By Codon-Based Mutagenesis,” J. Immunol. 155:1994-2004). CDR walking which randomizes the light chain can be used (see, Schier et al. (1996) “Isolation Of Picomolar Affinity Anti-C-Erbb-2 Single-Chain Fv By Molecular Evolution Of The Complementarity Determining Regions In The Center Of The Antibody Binding Site,” J. Mol. Biol. 263:551-567).

The disclosure thus contemplates the use of random mutagenesis to identify improved CDRs. Phage display technology can alternatively be used to increase (or decrease) CDR affinity. This technology, referred to as affinity maturation, employs mutagenesis or “CDR walking” and re-selection uses the target antigen or an antigenic fragment thereof to identify antibodies having CDRs that bind with higher (or lower) affinity to the antigen when compared with the initial or parental antibody (see, e.g., Glaser, S. M. et al. (1992) “Antibody Engineering By Codon-Based Mutagenesis In A Filamentous Phage Vector System,” J. Immunol. 149:3903-3913). Mutagenizing entire codons rather than single nucleotides results in a semi-randomized repertoire of amino acid mutations. Libraries can be constructed including of a pool of variant clones each of which differs by a single amino acid alteration in a single CDR and which contain variants representing each possible amino acid substitution for each CDR residue. Mutants with increased (or decreased) binding affinity for the antigen can be screened by contacting the immobilized mutants with labeled antigen. Any screening method known in the art can be used to identify mutant antibodies with increased (or decreased) avidity to the antigen (e.g., ELISA) (see, Wu, H. et al. (1998) “Stepwise In Vitro Affinity Maturation Of Vitaxin, An Alphav Beta3-Specific Humanized Mab,” Proc. Natl. Acad. Sci. (USA) 95(11):6037-6042; Yelton, D.E. et al. (1995) “Affinity Maturation Of The BR96Anti-Carcinoma Antibody By Codon-Based Mutagenesis,” J. Immunol. 155:1994-2004). CDR walking which randomizes the light chain can be used (see, Schier et al. (1996) “Isolation Of Picomolar Affinity Anti-C-Erbb-2 Single-Chain Fv By Molecular Evolution Of The Complementarity Determining Regions In The Center Of The Antibody Binding Site,” J. Mol. Biol. 263:551-567).

Methods for accomplishing such affinity maturation are described for example in: Krause, J. C. et al. (2011) “An Insertion Mutation That Distorts Antibody Binding Site Architecture Enhances Function Of A Human Antibody,” MBio. 2(1) pii: e00345-10. doi: 10.1128/mBio.00345-10; Kuan, C.T. et al. (2010) “Affinity-Matured Anti-Glycoprotein NMB Recombinant Immunotoxins Targeting Malignant Gliomas And Melanomas,” Int. J. Cancer 10.1002/ijc.25645; Hackel, B. J. et al. (2010) “Stability And CDR Composition Biases Enrich Binder Functionality Landscapes,” J. Mol. Biol. 401(1):84-96; Montgomery, D. L. et al. (2009) “Affinity Maturation And Characterization Of A Human Monoclonal Antibody Against HIV-1 gp41,” MAbs 1(5):462-474; Gustchina, E. et al. (2009) “Affinity Maturation By Targeted Diversification Of The CDR-H2 Loop Of A Monoclonal Fab Derived From A Synthetic Naïve Human Antibody Library And Directed Against The Internal Trimeric Coiled-Coil Of Gp41 Yields A Set Of Fabs With Improved HIV-1 Neutralization Potency And Breadth,” Virology 393(1):112-119; Finlay, W. J. et al. (2009) “Affinity Maturation Of A Humanized Rat Antibody For Anti-RAGE Therapy: Comprehensive Mutagenesis Reveals A High Level Of Mutational Plasticity Both Inside And Outside The Complementarity-Determining Regions,” J. Mol. Biol. 388(3):541-558; Bostrom, J. et al. (2009) “Improving Antibody Binding Affinity And Specificity For Therapeutic Development,” Methods Mol. Biol. 525:353-376; Steidl, S. et al. (2008) “In Vitro Affinity Maturation Of Human GM-CSF Antibodies By Targeted CDR-Diversification,” Mol. Immunol. 46(1):135-144; and Barderas, R. et al. (2008) “Affinity maturation of antibodies assisted by in silico modeling,” Proc. Natl. Acad. Sci. (USA) 105(26):9029-9034.

### 6. Exemplary Bispecific Antibody

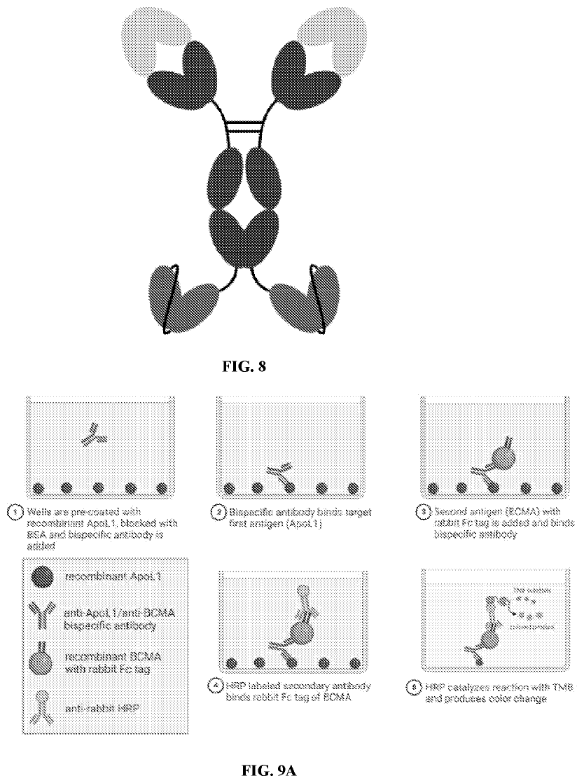

An anti-ApoL1, BCMA IgG1-scFv (Heavy Chain C-terminus) chimeric antibody has been designed and has the structure of FIG. 8.

The Fab portion is the heavy and light chain variable regions of recombinant clone SFIII 13.11 (anti-ApoL1) having the sequences SEQ ID NO:24 (VH) and SEQ ID NO:36 or SEQ ID NO:77 (VL).

The anti-BCMA is an ScFv of [clone 17A5], Human IgG1, Kappa fused to the Heavy Chain C-terminus of a human IgG1. The heavy and light chain variable sequences of anti-BCMA clone 17A5 are:

VH
(SEQ ID NO: 41)
EVQLLESGGGLVQPGGSLRLSCAASGFTFSSYAMSWVRQAPGKGLEWVSAI
SGSGGSTYYADSVKGRFTISRDNSKNTLYLQMNSLRAEDTAVYYCAKVAPY
FAPFDYWGQGTLVTVSS
VL
(SEQ ID NO: 42)
EIVLTQSPGTLSLSPGERATLSCRASQSVSSSYLAWYQQKPGQAPRLLIYG
ASSRATGIPDRFSGSGSGTDFTLTISRLEPEDFAVYYCQQYGNPPLYTFGQ
GTKVEIK

The CDR sequences of the anti-BCMA clone 17A5, bold in the sequences above are:

CDR1H:
(SEQ ID NO: 43)
SYAMS,
CDR2H:
(SEQ ID NO: 44)
AISGSGGSTYYADSVKG,
CDR3H:
(SEQ ID NO: 45)
VAPYFAPFDY,
CDR1L:
(SEQ ID NO: 46)
RASQSVSSSYLA,
CDR2L:
(SEQ ID NO: 47)
GASSRAT,
CDR3L:
(SEQ ID NO: 48)
QQYGNPPLYT.

An exemplary sequence for an anti-BCMA scFv is

(SEQ ID NO: 51)
EIVLTQSPGTLSLSPGERATLSCRASQSVSSSYLAWYQQKPGQAPRLLIYG
ASSRATGIPDRESGSGSGTDETLTISRLEPEDFAVYYCQQYGNPPLYTFGQ
GTKVEIKggggsggggsggggsEVQLLESGGGLVQPGGSLRLSCAASGFTF
SSYAMSWVRQAPGKGLEWVSAISGSGGSTYYADSVKGRFTISRDNSKNTLY
LQMNSLRAEDTAVYYCAKVAPYFAPFDYWGQGTLVTVSS,
where the CDRs are bold and ggggggggsggggs
(lowercase) (SEQ ID NO: 52) is a flexible linker.

In some embodiments, a bi-specific antibody includes the amino acid sequence

(SEQ ID NO: 71)
EVQLVESGGGLVKPGGSLKLSCAASGFTFSTYAMSWVRQSPEKRLEWVAEI
SNGGLYTYYPDTVTGRFTISRDNVKNILYLEMSSLRSEDTAIYYCIRENRN
WYFDLWGAGTTVTVSSASTKGPSVFPLAPSSKSTSGGTAALGCLVKDYFPE
PVTVSWNSGALTSGVHTFPAVLQSSGLYSLSSVVTVPSSSLGTQTYICNVN
HKPSNTKVDKKVEPKSCDKTHTCPPCPAPELLGGPSVFLFPPKPKDTLMIS
RTPEVTCVVVDVSHEDPEVKFNWYVDGVEVHNAKTKPREEQYNSTYRVVSV
LTVLHQDWLNGKEYKCKVSNKALPAPIEKTISKAKGQPREPQVYTLPPSRD
ELTKNQVSLTCLVKGFYPSDIAVEWESNGQPENNYKTTPPVLDSDGSFFLY
SKLTVDKSRWQQGNVFSCSVMHEALHNHYTQKSLSLSPGGGGSGGGGSGGG
GSEIVLTQSPGTLSLSPGERATLSCRASQSVSSSYLAWYQQKPGQAPRLLI
YGASSRATGIPDRFSGSGSGTDFTLTISRLEPEDFAVYYCQQYGNPPLYTF
GQGTKVEIKGGGGSGGGGSGGGGSEVQLLESGGGLVQPGGSLRLSCAASGF
TESSYAMSWVRQAPGKGLEWVSAISGSGGSTYYADSVKGRFTISRDNSKNT
LYLQMNSLRAEDTAVYYCAKVAPYFAPFDYWGQGTLVTVSS
or
a variant thereof with at least 70, 80, 90, or 95%
sequence identity thereto;
and/or
(SEQ ID NO: 72)
DVLMTQTPLSLPVSLGDQASISCRSSQSIVNSNGNTYLEWYLQKPGQSPKL
LIYKVSNRFSGVPDRFSGSGSGTDFTLKISRVEAEDLGVYYCFQGSHVPLT
FGAGTKLEIKRTVAAPSVFIFPPSDEQLKSGTASVVCLLNNFYPREAKVQW
KVDNALQSGNSQESVTEQDSKDSTYSLSSTLTLSKADYEKHKVYACEVTHQ
GLSSPVTKSFNRGEC
or
a variant thereof with at least 70, 80, 90, or 95%
sequence identity thereto.

With respect to SEQ ID NO:71,

(SEQ ID NO: 24)
EVQLVESGGGLVKPGGSLKLSCAASGFTFSTYAMSWVRQSPEKRLEWVAEI
SNGGLYTYYPDTVTGRFTISRDNVKNILYLEMSSLRSEDTAIYYCIRENRN
WYFDLWGAGTTVTVSS is the Heavy Chain Variable
Domain of STII 13.11 anti-ApoLl antibody;
(SEQ ID NO: 75)
ASTKGPSVFPLAPSSKSTSGGTAALGCLVKDYFPEPVTVSWNSGALTSGVH
TFPAVLQSSGLYSLSSVVTVPSSSLGTQTYICNVNHKPSNTKVDKKVEPKS
CDKTHTCPPCPAPELLGGPSVFLFPPKPKDTLMISRTPEVTCVVVDVSHED
PEVKFNWYVDGVEVHNAKTKPREEQYNSTYRVVSVLTVLHQDWLNGKEYKC
KVSNKALPAPIEKTISKAKGQPREPQVYTLPPSRDELTKNQVSLTCLVKGF
YPSDIAVEWESNGQPENNYKTTPPVLDSDGSFFLYSKLTVDKSRWQQGNVE
SCSVMHEALHNHYTQKSLSLSP is a heavy chain constant
region sequence;
(SEQ ID NO: 52)
GGGGSGGGGSGGGGS is a linker sequence;
and
(SEQ ID NO: 51)
EIVLTQSPGTLSLSPGERATLSCRASQSVSSSYLAWYQQKPGQAPRLLIYG
ASSRATGIPDRESGSGSGTDFTLTISRLEPEDFAVYYCQQYGNPPLYTFGQ
GTKVEIKGGGGSGGGGSGGGGSEQLLESGGGLVQPGGSLRLSCAASGFTF
SSYAMSWVRQAPGKGLEWVSAISGSGGSTYYADSVKGRFTISRDNSKNTLY
LQMNSLRAEDTAVYYCAKVAPYFAPFDYWGOGTLVTVSS is
(the variable domain of) BCMA 17A5 scFv.

With respect to SEQ ID NO:72:

(SEQ ID NO: 77)
DVLMTQTPLSLPVSLGDQASISCRSSQSIVNSNGNTYLEWYLQKPGQSP
KLLIYKVSNRFSGVPDRFSGSGSGTDFTLKISRVEAEDLGVYYCFQGSH
VPLTFGAGTKLEIK is the Light Chain Variable
Domain of STII 13.11 anti-ApoL 1 antibody;
and
(SEQ ID NO: 76)
RTVAAPSVFIFPPSDEQLKSGTASVVCLLNNFYPREAKVQWKVDNALQS
GNSQESVTEQDSKDSTYSLSSTLTLSKADYEKHKVYACEVTHQGLSSPV
TKSFNRGEC is a light chain constant region
sequence.

A bispecific antibody having the structure of FIG. 8 can be formed by two copies each of the amino acid sequences SEQ ID NO:71 and SEQ ID NO:72, e.g., upon co-expression of nucleic acids encoding the amino acid sequences of SEQ ID NO:71 (e.g., SEQ ID NO:73) and SEQ ID NO:72 (e.g., SEQ ID NO:74).

For BMCA clone 17A5 sequences, and alternative anti-BCMA sequences, see WO 2014/122144, which is specifically incorporated by reference herein in its entirety, and which sequences can be used to derive alternative anti-BMCA arms of the antibody.

In another embodiments, in additional or alternative to anti-ApoL1, the Fab portion includes the heavy and light chain CDRs, or the entire heavy and light chain variable regions are of recombinant clone SFII 134.3 (anti-Hpr) or SFII 14.11 (anti-Hpr) having the sequences SEQ ID NO:3 (VH) and SEQ ID NO:14 (VL) or the sequences SEQ ID NO:56 (VH) and SEQ ID NO:65 (VL), respectively and associated CDRs thereof as discussed in more detail elsewhere herein.

### 7. Other Exemplary Antibodies for Targeting

As introduced above, cell specific markers and cancer antigens are preferred targets of targeting moieties, preferably antibodies. Antibodies for targeting cell specific markers and cancer antigens are known and the art and can be used in the disclosed compositions.

A specific example of an anti-B7-H1 (PD-L1) antibody is MDX-1105 (WO/2007/005874, published 1 1 Jan. 2007)), a human anti-B7-H1 antibody.

For anti-B7-DC (PD-L2) antibodies see 7,411,051, 7,052,694, 7,390,888, and U.S. Published Application No. 2006/0099203.

An example of an anti-CTLA4 antibody contemplated for use in disclosed compositions and methods include an antibody as described in PCT/US2006/043690 (Fischkoff et al, WO/2007/056539).

Other specific exemplary antibodies who's sequences can utilized in the disclosed bi-specific and multispecific antibodies include, but are not limited to, Talquetamab (JNJ64407564), Indatuximab ravtansine, Daratumumab, Elotuzumab, DFRF4539A, and BFCR4350A.

Daratumumab is a human IgG(kappa) antibody that targets a unique epitope of CD38 (FDA approved in 2016).

Indatuximab ravtansine target CD138.

Elotuzumab is a mAb aimed at an extracellular domain of SLAMF7 and has shown moderate success in a Phase 3 ELOQUENT-2 study.

DFRF4539A and BFCR4350A target FcRH5.

Talquetamab is a bispecific antibody that targets GPRC5D on multiple myeloma and CD3 on T cells.

See, e.g., Leow et al., J Pers Med. 11(5):334 (2021). doi: 10.3390/jpm11050334 and U.S. Pat. No. 10,562,968.

An antibody that targets Claudin 18.2 antibody is provided in WO 2022/136642A1.

An antibody that targets MUC1 (Gatipotuzumab) is provided in e.g., WO 2019/219891 and KR20210010565A.

Examples of antibodies that target MSLN (Mesothelin) antibodies include, but are not limited to,
Anetumab ravtansine, see, e.g., W02009/068204A1 and WO2020234114A1;
SS1P, see, e.g., U.S. Pat. No. 8,460,660B2;
Amatuximab or MORAb-009, see, e.g., U.S. Pat. No. 9,803,022; and
SD1/SD2, see, e.g., WO 2014/052064.

An example of an antibody that targets PMEL17 is discussed in, e.g., U.S. Pat. No. 9,056,910, WO 2013/165940, EP 2844300.

All of the foregoing patents and applications are specifically incorporated by reference in their entireties, including, but not limited to, their antibody sequences, most particularly the CDRs, which can be incorporated into the disclosed the compositions for targeting ApoL1 to cells expressing their target antigen.

### B. Compositions for Targeting Exogenous ApoL1

Compositions for increasing the cellular internalization of exogenous ApoL1 are also provided. Although not necessarily used exclusively for this purpose, the compositions can be used to deliver exogenous ApoL1, e.g., recombinant ApoL1, into cells.

The compositions typically include ApoL1, such as the ApoL1 of SEQ ID NO:50, or a functional fragment or variant thereof having, for example, 70%, 75%, 80%, 85%, 90%, 95% sequence identity thereto. The compositions typically further include a targeting moiety that binds to a cell specific marker, such as a cancer antigen, present on cells, thus facilitating targeting of the ApoL1 or fragment or variant thereof to the target cells. The targeted ApoL1 can be internalized by cells in an effective amount to increase cell death of the target cells. The ApoL1 or fragment or variant thereof can be fused or directly or indirectly conjugated to the targeting moiety. For example, in some embodiments the ApoL1 is a fusion protein including a ApoL1 or fragment or variant thereof and a targeting moiety. In some embodiments, the ApoL1 or fragment or variant thereof is conjugated to the targeting moiety. In some embodiments, the ApoL1 or fragment or variant thereof is packaged in a delivery vehicle such as nanoparticles or liposomes and the delivery vehicle further has a targeting moiety conjugated thereto.

Any of the compositions can further include a cell penetrating peptide.

### 1. Targeting Moieties

Representative targeting moieties include, but are not limited to, antibodies and antigen binding fragments thereof, aptamers, peptides, and small molecules. The binding moiety can be conjugated to a polymer that forms the nanocarrier. Typically the binding moiety is displayed on the outer shell of the nanocarrier. The outer shell can serve as a shield to prevent the nanocarrier from being recognized by a subject's immune system thereby increasing the half-life of the nanocarrier in the subject. The nanoparticles can contain a hydrophobic core. In the case of liposomal nanoparticles the core can also be hydrophilic. In some embodiments, the hydrophobic core is made of a biodegradable polymeric material. The inner core carries therapeutic payloads and releases the therapeutic payloads at a sustained rate after systemic, intraperitoneal, oral, pulmonary, or topical administration. The nanocarrier also optionally include a detectable label, for example a fluorophore or NMR contrast agent that allows visualization of nanocarriers.

In other embodiments, the targeting moiety is conjugated, linked, or directly fused to the ApoL1 or fragment or variant thereof.

The targeting moiety of the nanocarrier can be an antibody or antigen binding fragment thereof. The targeting moieties should have an affinity for a cell-surface receptor or cell-surface antigen on the target cells. The targeting moieties may result in internalization of the nanocarrier within the target cell.

The targeting moiety can specifically recognize and bind to a target molecule specific for a cell type, a tissue type, or an organ. The target molecule can be a cell surface polypeptide, lipid, or glycolipid. The target molecule can be a receptor that is selectively expressed on a specific cell surface, a tissue or an organ. Cell specific markers can be for specific types of cells including, but not limited to stem cells, skin cells, blood cells, immune cells, muscle cells, nerve cells, cancer cells, virally infected cells, bacterial cells, fungal cells, organ specific cells, and other eukaryotic cells. The cell markers can be specific for endothelial, ectodermal, or mesenchymal cells. Representative cell specific markers include, but are not limited to, cancer specific markers. The cell markers can be any of the cell specific marker, including cancer and tumor antigens and other mammalian and non-mammalian cell targets (e.g., bacterial and fungal cells) including, but not limited to, those provided elsewhere herein (see, e.g., above). Typically the targeting moiety does not target a trypanosome specific surface antigen.

The targeting moiety can be a peptide. The targeting peptides can be covalently associated with the polymer and the covalent association can be mediated by a linker. The targeting moiety can be antibody or antigen binding fragment of fusion protein thereof. The antibody can be in any format, including, but not limited to those provided elsewhere herein (see, e.g., above).

### 2. Exemplary Nanocarriers

Nanocarrier compositions including ApoL1 or a fragment or variant thereof and targeting moiety loaded into, attached to the surface of, and/or enclosed within a delivery vehicle, are provided.

The nanocarrier delivery vehicles can be, for example, polymeric particles, inorganic particles, silica particles, liposomes, micelles, multilamellar vesicles, or microbubbles.

In some embodiments, the delivery vehicles are nanoscale compositions, for example, 10 nm up to, but not including, about 1 micron. However, it will be appreciated that in some embodiments, and for some uses, the particles can be smaller, or larger (e.g., microparticles, etc.). Although many of the compositions disclosed herein are referred to as nanoparticle or nanocarrier compositions, it will be appreciated that in some embodiments and for some uses the carrier can be somewhat larger than nanoparticles. For example, carrier compositions can be between about 1 micron to about 1000 microns. Such compositions can be referred to as microparticulate compositions. For example, a nanocarriers according to the present disclosure may be a microparticle. Microparticles can a diameter between, for example, 0.1 and 100 m in size. In another example, the nanocarriers may be a supraparticle. Supraparticles are particles having a diameter above about 100 μm in size. For example, supraparticle may have a diameter of about 100 μm to about 1,000 μm in size.

Microbubbles are bubbles smaller than one millimetre in diameter, but larger than one micrometer with widespread application in industry, life science, and medicine. The composition of the bubble shell and filling material determine empart characteristics such as buoyancy, crush strength, thermal conductivity, and acoustic properties. In medicine they have applications in diagnostics such as imaging and therapeutics such as drug delivery.

In some embodiments for treating cancer it is desirable that the particle be of a size suitable to access the tumor microenvironment. In particular embodiments, the particle is of a size suitable to access the tumor microenvironment and/or the tumor cells by enhanced permeability and retention (EPR) effect. EPR refers to the property by which certain sizes of molecules tend to accumulate in tumor tissue much more than they do in normal tissues. Therefore, in an exemplary treatment of cancer, the delivery vehicle can be in the range of about 25 nm to about 500 nm inclusive, or in the range of about 50 nm to about 300 nm inclusive. In another example, the delivery vehicle can be in the range of about 80 nm to about 120 nm inclusive. In another example, the delivery vehicle can be in the range of about 85 nm to about 110 nm inclusive.

The polymeric nanoparticles are typically formed using an emulsion process, single or double, using an aqueous and a non-aqueous solvent. Typically, the nanoparticles contain a minimal amount of the non-aqueous solvent after solvent removal.

In one embodiment, nanoparticles are prepared using emulsion solvent evaporation method. A polymeric material is dissolved in a water immiscible organic solvent and mixed with a drug solution or a combination of drug solutions. The water immiscible organic solvent can be a GRAS ingredient such as chloroform, dichloromethane, and acyl acetate. The drug can be dissolved in, but is not limited to, one or a plurality of the following: acetone, ethanol, methanol, isopropyl alcohol, acetonitrile and Dimethyl sulfoxide (DMSO). An aqueous solution is then added into the resulting mixture solution to yield emulsion solution by emulsification. The emulsification technique can be, but not limited to, probe sonication or homogenization through a homogenizer.

In another embodiment, nanoparticles are prepared using nanoprecipitation methods or microfluidic devices. A polymeric material is mixed with a drug or drug combinations in a water miscible organic solvent. The water miscible organic solvent can be one or more of the following: acetone, ethanol, methanol, isopropyl alcohol, acetonitrile and Dimethyl sulfoxide (DMSO). The resulting mixture solution is then added to an aqueous solution to yield nanoparticle solution. The agents may be associated with the surface of, encapsulated within, surrounded by, and/or distributed throughout the polymeric matrix of the particles.

In another embodiment, nanoparticles are prepared by the self-assembly of the amphiphilic polymers, optionally including hydrophilic and/or hydrophobic polymers, using emulsion solvent evaporation, a single-step nanoprecipitation method, or microfluidic devices.

Other exemplary methods of producing nanoparticles encompassed by the present disclosure are described in Zhou, et al., Biomaterials, 33(2):583-591 (2012) and Han, et al., Nanomedicine (2016).

Two methods to incorporate targeting moieties into the nanoparticles include: i) conjugation of targeting ligands to the hydrophilic region (e.g. PEG) of polymers prior to nanoparticle preparation; and ii) incorporation of targeting molecules onto nanoparticles where the PEG layer on the nanoparticle surface can be cleaved in the presence of a chemical or enzyme at tissues of interest to expose the targeting molecules.

Particles may be microparticles or nanoparticles. Nanoparticles are often utilized for intertissue application, penetration of cells, and certain routes of administration. The nanoparticles may have any desired size for the intended use. The nanoparticles may have any diameter from 10 nm up to about 1,000 nm. The nanoparticle can have a diameter from 10 nm to 900 nm, from 10 nm to 800 nm, from 10 nm to 700 nm, from 10 nm to 600 nm, from 10 nm to 500 nm, from 20 nm from 500 nm, from 30 nm to 500 nm, from 40 nm to 500 nm, from 50 nm to 500 nm, from 50 nm to 400 nm, from 50 nm to 350 nm, from 50 nm to 300 nm, or from 50 nm to 200 nm. In some embodiments the nanoparticles can have a diameter less than 400 nm, less than 300 nm, or less than 200 nm. The range can be between 50 nm and 300 nm.

The average diameters of the nanoparticles are typically between about 50 nm and about 500 nm, or between about 50 nm and about 350 nm. In some embodiments, the average diameters of the nanoparticles are about 100 nm. The zeta potential of the nanoparticles is typically between about −50 mV and about +50 mV, or between about −25 mV and +25 mV, or between about −10 mV and about +10 mv.

In some embodiments, the particles are brain-penetrating polymeric nanoparticles that can be loaded with drugs and optimized for intracranial convection-enhanced delivery (CED) such as those discussed in WO 2013/166487 and U.S. Published Application No. 2015/0118311. For example, the particles can be formed by emulsifying a polymer-drug solution, then removing solvent and centrifuging at a first force to remove the larger particles, then collecting the smaller particles using a second higher force to sediment the smaller particles having a diameter of less than 100 nm, or in the range of 25-75 nanometers average diameter, able to penetrate brain interstitial spaces.

Partially water-miscible organic solvents, such as benzyl alcohol, butyl lactate, and ethyl acetate (EA), allow nanoparticle formulation through an emulsion-diffusion mechanism and are able to produce smaller nanoparticles than water-immiscible solvents such as dichloromethane (DCM). Using partially water-miscible organic solvents improves the yield of brain-penetrating nanoparticles. Representative solvents that can be used include DCM, benzyl alcohol, butyl lactate, and ethyl acetate (EA), acetone. EA is particularly attractive because of its low toxicity.

To reduce aggregation, a sugar such as the FDA-approved disaccharide trehalose can added to the composition. Other sugars include glucose, sucrose and lactose. Typically, the weight ratio of sugar to nanoparticles is between 10-50%.

### a. Polymers

The nanocarrier can be a particle containing one or more hydrophilic polymers. Hydrophilic polymers include cellulosic polymers such as starch and polysaccharides; hydrophilic polypeptides; poly(amino acids) such as poly-L-glutamic acid (PGS), gamma-polyglutamic acid, poly-L-aspartic acid, poly-L-serine, or poly-L-lysine; polyalkylene glycols and polyalkylene oxides such as polyethylene glycol (PEG), polypropylene glycol (PPG), and poly(ethylene oxide) (PEO); poly(oxyethylated polyol); poly(olefinic alcohol); polyvinylpyrrolidone); poly(hydroxyalkylmethacrylamide); poly(hydroxyalkylmethacrylate); poly(saccharides); poly(hydroxy acids); poly(vinyl alcohol), and copolymers thereof.

The nanoparticle can contain one or more hydrophobic polymers. Examples of suitable hydrophobic polymers include polyhydroxyacids such as poly(lactic acid), poly(glycolic acid), and poly(lactic acid-co-glycolic acids); polyhydroxyalkanoates such as poly3-hydroxybutyrate or poly4-hydroxybutyrate; polycaprolactones; poly(orthoesters); polyanhydrides; poly(phosphazenes); poly(lactide-co-caprolactones); polycarbonates such as tyrosine polycarbonates; polyamides (including synthetic and natural polyamides), polypeptides, and poly(amino acids); polyesteramides; polyesters; poly(dioxanones); poly(alkylene alkylates); hydrophobic polyethers; polyurethanes; polyetheresters; polyacetals; polycyanoacrylates; polyacrylates; polymethylmethacrylates; polysiloxanes; poly(oxyethylene)/poly(oxypropylene) copolymers; polyketals; polyphosphates; polyhydroxyvalerates; polyalkylene oxalates; polyalkylene succinates; poly(maleic acids), as well as copolymers thereof.

In certain embodiments, the hydrophobic polymer is an aliphatic polyester. In some embodiments, the hydrophobic polymer is poly(lactic acid), poly(glycolic acid), or poly(lactic acid-co-glycolic acid).

The nanoparticle can contain one or more biodegradable polymers. Biodegradable polymers can include polymers that are insoluble or sparingly soluble in water that are converted chemically or enzymatically in the body into water-soluble materials. Biodegradable polymers can include soluble polymers crosslinked by hydolyzable cross-linking groups to render the crosslinked polymer insoluble or sparingly soluble in water.

Biodegradable polymers in the nanoparticle can include polyamides, polycarbonates, polyalkylenes, polyalkylene glycols, polyalkylene oxides, polyalkylene terepthalates, polyvinyl alcohols, polyvinyl ethers, polyvinyl esters, polyvinyl halides, polyvinylpyrrolidone, polyglycolides, polysiloxanes, polyurethanes and copolymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, poly (methyl methacrylate), poly(ethylmethacrylate), poly(butylmethacrylate), poly(isobutylmethacrylate), poly(hexlmethacrylate), poly(isodecylmethacrylate), poly(lauryl methacrylate), poly (phenyl methacrylate), poly(methyl acrylate), poly(isopropyl acrylate), poly(isobutyl acrylate), poly(octadecyl acrylate), polyethylene, polypropylene poly(ethylene glycol), poly(ethylene oxide), poly(ethylene terephthalate), poly(vinyl alcohols), poly(vinyl acetate, poly vinyl chloride polystyrene and polyvinylpryrrolidone, derivatives thereof, linear and branched copolymers and block copolymers thereof, and blends thereof. Exemplary biodegradable polymers include polyesters, poly(ortho esters), poly(ethylene imines), poly(caprolactones), poly(hydroxybutyrates), poly(hydroxyvalerates), polyanhydrides, poly(acrylic acids), polyglycolides, poly(urethanes), polycarbonates, polyphosphate esters, polyphosphazenes, derivatives thereof, linear and branched copolymers and block copolymers thereof, and blends thereof.

The nanoparticles can contain one or more amphiphilic polymers. Amphiphilic polymers can be polymers containing a hydrophobic polymer block and a hydrophilic polymer block. The hydrophobic polymer block can contain one or more of the hydrophobic polymers above or a derivative or copolymer thereof. The hydrophilic polymer block can contain one or more of the hydrophilic polymers above or a derivative or copolymer thereof. In some embodiments the amphiphilic polymer is a di-block polymer containing a hydrophobic end formed from a hydrophobic polymer and a hydrophilic end formed of a hydrophilic polymer. In some embodiments, a moiety can be attached to the hydrophobic end, to the hydrophilic end. or both.

In some embodiments the nanoparticles contain a first amphiphilic polymer having a hydrophobic polymer block, a hydrophilic polymer block, and targeting moiety conjugated to the hydrophilic polymer block; and a second amphiphilic polymer having a hydrophobic polymer block and a hydrophilic polymer block but without the targeting moiety. The hydrophobic polymer block of the first amphiphilic polymer and the hydrophobic polymer block of the second amphiphilic polymer may be the same or different. Likewise, the hydrophilic polymer block of the first amphiphilic polymer and the hydrophilic polymer block of the second amphiphilic polymer may be the same or different.

In some embodiments the nanoparticle contains biodegradable polyesters or polyanhydrides such as poly(lactic acid), poly(glycolic acid), and poly(lactic-co-glycolic acid). The nanoparticles can contain one more of the following polyesters: homopolymers including glycolic acid units, referred to herein as “PGA”, and lactic acid units, such as poly-L-lactic acid, poly-D-lactic acid, poly-D,L-lactic acid, poly-L-lactide, poly-D-lactide, and poly-D,L-lactide, collectively referred to herein as “PLA”, and caprolactone units, such as poly(ε-caprolactone), collectively referred to herein as “PCL”; and copolymers including lactic acid and glycolic acid units, such as various forms of poly(lactic acid-co-glycolic acid) and poly(lactide-co-gly colide) characterized by the ratio of lactic acid:glycolic acid, collectively referred to herein as “PLGA”; and polyacrylates, and derivatives thereof. Exemplary polymers also include copolymers of polyethylene glycol (PEG) and the aforementioned polyesters, such as various forms of PLGA-PEG or PLA-PEG copolymers, collectively referred to herein as “PEGylated polymers”. In certain embodiments, the PEG region can be covalently associated with polymer to yield “PEGylated polymers” by a cleavable linker. Other polymers include PLGA- poly(ε-carbobenzoxyl-L-lysine) (PLL) (i.e., PLGA-PLL).

The nanoparticles can also contain one or more polymer conjugates containing end-to-end linkages between the polymer and a targeting moiety or a detectable label. For example, a modified polymer can be a PLGA-PEG-peptide block polymer.

The nanoparticles can contain one or a mixture of two or more polymers. The nanoparticles may contain other entities such as stabilizers, surfactants, or lipids. The nanoparticles may contain a first polymer having a targeting moiety and a second polymer not having the targeting moiety. By adjusting the ratio of the targeted and non-targeted polymers, the density of the targeting moiety on the exterior of the particle can be adjusted.

The nanoparticles can contain an amphiphilic polymer having a hydrophobic end, a hydrophilic end, and a targeting moiety attached to the hydrophilic end. In some embodiments the amphiphilic macromolecule is a block copolymer having a hydrophobic polymer block, a hydrophilic polymer block covalently coupled to the hydrophobic polymer block, and a targeting moiety covalently coupled to the hydrophilic polymer block. For example, the amphiphilic polymer can have a conjugate having the structure A-B-X where A is a hydrophobic molecule or hydrophobic polymer, B is a hydrophilic molecule or hydrophilic polymer, and X is a targeting moiety. Exemplary amphiphilic polymers include those where A is a hydrophobic biodegradable polymer, B is PEG, and X is a targeting moiety that targets, binds.

In some embodiments the nanoparticle contains a first amphiphilic polymer having the structure A-B-X as described above and a second amphiphilic polymer having the structure A-B, where A and B in the second amphiphilic macromolecule are chosen independently from the A and B in the first amphiphilic macromolecule, although they may be the same.

### b. Liposomes and Micelles

In some embodiments, the nanocarrier is a liposome or micelle. Liposomes are spherical vesicles composed of concentric phospholipid bilayers separated by aqueous compartments. Liposomes can adhere to and form a molecular film on cellular surfaces. Structurally, liposomes are lipid vesicles composed of concentric phospholipid bilayers which enclose an aqueous interior (Gregoriadis, et al., Int. J. Pharm., 300, 125-30 2005; Gregoriadis and Ryman, Biochem. J, 124, 58P (1971)). Hydrophobic compounds associate with the lipid phase, while hydrophilic compounds associate with the aqueous phase.

Liposomes have the ability to form a molecular film on cell and tissue surfaces. Clinical studies have proven the efficacy of liposomes as a topical healing agent (Dausch, et al., Klin Monatsbl Augenheilkd 223, 974-83 (2006); Lee, et al., Kin Monatsbl Augenheilkd 221, 825-36 (2004)). Liposomes have also been used in ophthalmology to ameliorate keratitis, corneal transplant rejection, uveitis, endophthalmitis, and proliferative vitreoretinopathy (Ebrahim, et al., 2005; Li, et al., 2007).

Liposomes have been widely studied as drug carriers for a variety of chemotherapeutic agents (approximately 25,000 scientific articles have been published on the subject) (Gregoriadis, N Engl J Med 295, 765-70 (1976); Gregoriadis, et al., Int. J Pharm. 300, 125-30 (2005)). Water-soluble anticancer substances such as doxorubicin can be protected inside the aqueous compartment(s) of liposomes delimited by the phospholipid bilayer(s), whereas fat-soluble substances such as amphotericin and capsaicin can be integrated into the phospholipid bilayer (Aboul-Fadl, Curr Med Chem 12, 2193-214 (2005); Tyagi, et al., J Urol 171, 483-9 (2004)). Topical and vitreous delivery of cyclosporine was drastically improved with liposomes (Lallemand, et al., Eur J Pharm Biopharm 56, 307-18 2003). Delivery of chemotherapeutic agents lead to improved pharmacokinetics and reduced toxicity profile (Gregoriadis, Trends Biotechnol 13, 527-37 (1995); Gregoriadis and Allison, FEBS Lett 45, 71-4 1974; Sapra, et al., Curr Drug Deliv 2, 369-81 (2005)). More than ten liposomal and lipid-based formulations have been approved by regulatory authorities and many liposomal drugs are in preclinical development or in clinical trials (Barnes, Expert Opin Pharmacother 7, 607-15 (2006); Minko, et al., Anticancer Agents Med Chem 6, 537-52 (2006)). The safety data with respect to acute, subchronic, and chronic toxicity of liposomes has been assimilated from the vast clinical experience of using liposomes in the clinic for thousands of patients.

Nanocarriers such as liposomes and micelles can be formed from one or more lipids, which can be neutral, anionic, or cationic at physiologic pH. Suitable neutral and anionic lipids include, but are not limited to, sterols and lipids such as cholesterol, phospholipids, lysolipids, lysophospholipids, sphingolipids or pegylated lipids. Neutral and anionic lipids include, but are not limited to, phosphatidylcholine (PC) (such as egg PC, soy PC), including, but limited to, 1,2-diacyl-glvcero-3-phosphocholines; phosphatidylserine (PS), phosphatidylglycerol, phosphatidylinositol (PI); glycolipids; sphingophospholipids such as sphingomyelin and sphingoglycolipids (also known as 1-ceramidyl glucosides) such as ceramide galactopyranoside, gangliosides and cerebrosides; fatty acids, sterols, containing a carboxylic acid group for example, cholesterol; 1,2-diacyl-sn-glycero-3-phosphoethanolamine, including, but not limited to, 1,2-dioleylphosphoethanolamine (DOPE), 1,2-dihexadecylphosphoethanolamine (DHPE), 1,2-distearoylphosphatidylcholine (DSPC), 1,2-dipalmitoyl phosphatidylcholine (DPPC), and 1,2-dimyristoylphosphatidylcholine (DMPC). The lipids can also include various natural (e.g., tissue derived L-α-phosphatidyl: egg yolk, heart, brain, liver, soybean) and/or synthetic (e.g., saturated and unsaturated 1,2-diacyl-sn-glycero-3-phosphocholines, 1-acyl-2-acyl-sn-glycero-3-phosphocholines, 1,2-diheptanoyl-SN-glycero-3-phosphocholine) derivatives of the lipids. In some embodiments, the liposomes contain a phosphaditylcholine (PC) head group, and optionally sphingomyelin. In another embodiment, the liposomes contain DPPC. In a further embodiment, the liposomes contain a neutral lipid, such as 1,2-dioleoylphosphatidylcholine (DOPC).

In certain embodiments, the liposomes are generated from a single type of phospholipid. In some embodiments, the phospholipid has a phosphaditylcholine head group, and, can be, for example, sphingomyelin. The liposomes may include a sphingomyelin metabolite. Sphingomyelin metabolites used to formulate the liposomes include, without limitation, ceramide, sphingosine, or sphingosine 1-phosphate. The concentration of the sphingomyelin metabolites included in the lipids used to formulate the liposomes can range from about 0.1 mol % to about 10 mol %, or from about 2.0 mol % to about 5.0 mol %, or can be in a concentration of about 1.0 mol %.

Suitable cationic lipids in the liposomes include, but are not limited to, N-[1-(2,3-dioleoyloxy)propyl]-N,N,N-trimethyl ammonium salts, also references as TAP lipids, for example methylsulfate salt. Suitable TAP lipids include, but are not limited to, DOTAP (dioleoyl-), DMTAP (dimyristoyl-), DPTAP (dipalmitoyl-), and DSTAP (distearoyl-). Suitable cationic lipids in the liposomes include, but are not limited to, dimethyldioctadecyl ammonium bromide (DDAB), 1,2-diacyloxy-3-trimethylammonium propanes, N-[1-(2,3-dioloyloxy)propyl]-N,N-dimethyl amine (DODAP), 1,2-diacyloxy-3-dimethylammonium propanes, N-[1-(2,3-dioleyloxy)propyl]-N,N,N-trimethylammonium chloride (DOTMA). 1,2-dialkyloxy-3-dimethylammonium propanes, dioctadecylamidoglycylspermine (DOGS), 3-[N-(N′,N′-dimethylammo-ethane)carbamoyl]cholesterol (DC-Chol); 2,3-dioleoyloxy-N-(2-(sperminecarboxamido)-ethyl)-N,N-dimethyl-1-propanaminium trifluoro-acetate (DOSPA), β-alanyl cholesterol, cetyl trimethyl ammonium bromide (CTAB), diC₁₄-amidine, N-ferf-butyl-N′-tetradecyl-3-tetradecylamino-propionamidine, N-(alpha-trimethylammonioacetyl)didodecyl-D-glutamate chloride (TMAG), ditetradecanoyl-N-(trimethylammonio-acetyl)diethanolamine chloride, 1,3-dioleoyloxy-2-(6-carboxy-spermyl)-propylamide (DOSPER), and N, N, N′, N′-tetramethyl-, N′-bis(2-hydroxylethyl)-2,3-dioleoyloxy-1,4-butanediammonium iodide. In one embodiment, the cationic lipids can be 1-[2-(acyloxy)ethyl]2-alkyl(alkenyl)-3-(2-hydroxyethyl)-imidazolinium chloride derivatives, for example, 1-[2-(9(Z)-octadecenoyloxy)ethyl]-2-(8(Z)-heptadecenyl-3-(2-hydroxyethyl)imidazolinium chloride (DOTIM), and 1-[2-(hexadecanoyloxy)ethyl]-2-pentadecyl-3-(2-hydroxyethyl)imidazolinium chloride (DPTIM). In one embodiment, the cationic lipids can be 2,3-dialkyloxypropyl quintenary ammonium compound derivatives containing a hydroxyalkyl moiety on the quaternary amine, for example, 1,2-dioleoyl-3-dimethyl-hydroxyethyl ammonium bromide (DORI), 1,2-dioleyloxypropyl-3-dimethyl-hydroxyethyl ammonium bromide (DORIE), 1,2-dioleyloxypropyl-3-dimetyl-hydroxypropyl ammonium bromide (DORIE-HP), 1,2-dioleyl-oxy-propyl-3-dimethyl-hydroxybutyl ammonium bromide (DORTE-HB), 1,2-dioleyloxypropyl-3-dimethyl-hydroxypentyl ammonium bromide (DORIE-Hpe), 1,2-dimyristyloxypropyl-3-dimethyl-hydroxylethyl ammonium bromide (DMRIE), 1,2-dipalmityloxypropyl-3-dimethyl-hydroxyethyl ammonium bromide (DPRIE), and 1,2-disteryloxypropyl-3-dimethyl-hydroxyethyl ammonium bromide (DSRIE).

The lipids may be formed from a combination of more than one lipid, for example, a charged lipid may be combined with a lipid that is non-ionic or uncharged at physiological pH. Non-ionic lipids include, but are not limited to, cholesterol and DOPE (1,2-dioleolylglyceryl phosphatidylethanolamine). The molar ratio of a first phospholipid, such as sphingomyelin, to second lipid can range from about 5:1 to about 1:1 or 3:1 to about 1:1, or from about 1.5:1 to about 1:1, or the molar ratio is about 1:1.

In some embodiments, liposomes or micelles include phospholipids, cholesterols and nitrogen-containing lipids. Examples include phospholipids, including natural phospholipids such as phosphatidylcholine, phosphatidylserine, phosphatidylglycerol, phosphatidylinositol, phosphatidylethanolamine, phosphatidic acid, cardiolipin, sphingomyelin, egg yolk lecithin, soybean lecithin, and lysolecithin, as well as hydrogenated products thereof obtained in a standard manner. It is also possible to use synthetic phospholipids such as dicetyl phosphate, distearoylphosphatidylcholine, dipalmitoylphosphatidylcholine, dipalmitoylphosphatidylethanolamine, dipalmitoylphosphatidylserine, eleostearoylphosphatidylcholine, eleostearoylphosphatidylethanolamine as well as homo-poly{N′-[N-(2-aminoethyl)-2-aminoethyl]aspartamide}P[Asp(DET)] and block-catiomer poly(ethyleneglycol) (PEG)-b-P[Asp(DET)].

In some embodiments, the liposomes are long circulating liposomes or stealth liposomes such as those reviewed in Immordino, et al, Int J Nanomedicine, 1(3):297-315 (2006)), which is specifically incorporated by reference herein in its entirety. For example, liposomes have been developed with surfaces modified with a variety of molecules including glycolipids and sialic acid. Long-circulating liposomes can include, for example, synthetic polymer poly-(ethylene glycol) (PEG) in liposome composition. The PEG on the surface of the liposomal carrier can extend blood-circulation time while reducing mononuclear phagocyte system uptake (stealth liposomes) and serve as an anchor for the targeting moiety.

Antibodies and antibody fragments are widely employed for targeting moieties for liposomes due to the high specificity for their target antigens. Referred to immunoliposomes, methods of generated targeted liposomes by coupling of antibodies to the liposomal surface are known in the art. Such techniques include, but are not limited to, conventional coupling and maleimide based techniques. See, for example, (Paszko and Senge, Curr Med Chem., 19(31):5239-77 (2012), Kelly, et al., Journal of Drug Delivery, Volume 2011 (2011), Article ID 727241, 11 pages).

The micelles can be polymer micelles, for example, those composed of amphiphilic di- or tri-block copolymers made of solvophilic and solvophobic blocks (see, e.g., Croy and Kwon, Curr Pharm Des., 12(36):4669-84 (2006)).

### 3. Other Functional Elements

Other functional elements that can be associated with, linked, conjugated, or otherwise attached directly or indirectly to the ApoL1, antibodies, or to a particle or other delivery vehicle thereof, include protein transduction domains and fusogenic peptides.

For example, the efficiency of particle delivery systems can also be improved by the attachment of functional ligands to the particle surface. Potential ligands include, but are not limited to, small molecules, cell-penetrating peptides (CPPs), targeting peptides, antibodies or aptamers (Yu, et al., PLoS One., 6:e24077 (2011), Cu, et al., J Control Release, 156:258-264 (2011), Nie, et al., J Control Release, 138:64-70 (2009), Cruz, et al., J Control Release, 144:118-126 (2010)). Attachment of these moieties serves a variety of different functions; such as inducing intracellular uptake, endosome disruption, and delivery of the plasmid payload to the nucleus. There have been numerous methods employed to tether ligands to the particle surface. One approach is direct covalent attachment to the functional groups on PLGA NPs (Bertram, Acta Biomater. 5:2860-2871 (2009)). Another approach utilizes amphiphilic conjugates like avidin palmitate to secure biotinylated ligands to the NP surface (Fahmy, et al., Biomaterials, 26:5727-5736 (2005), Cu, et al., Nanomedicine, 6:334-343 (2010)). This approach produces particles with enhanced uptake into cells, but reduced pDNA release and gene transfection, which is likely due to the surface modification occluding pDNA release. In a similar approach, lipid-conjugated polyethylene glycol (PEG) is used as a multivalent linker of penetratin, a CPP, or folate (Cheng, et al., Biomaterials, 32:6194-6203 (2011)).

These methods, as well as other methods discussed herein, and others methods known in the art, can be combined to tune particle function and efficacy. In some preferred embodiments, PEG is used as a linker for linking functional molecules to particles. For example, DSPE-PEG(2000)-maleimide is commercially available and can be used utilized for covalently attaching functional molecules such as CPP.

“Protein Transduction Domain” or PTD refers to a polypeptide, polynucleotide, or organic or inorganic compounds that facilitates traversing a lipid bilayer, micelle, cell membrane, organelle membrane, or vesicle membrane. A PTD attached to another molecule facilitates the molecule traversing membranes, for example going from extracellular space to intracellular space, or cytosol to within an organelle. PTA can be short basic peptide sequences such as those present in many cellular and viral proteins. Exemplary protein transduction domains that are well-known in the art include, but are not limited to, the Antennapedia PTD and the TAT (transactivator of transcription) PTD, poly-arginine, poly-lysine or mixtures of arginine and lysine, HIV TAT (YGRKKRRQRRR (SEQ ID NO:53) or RKKRRQRRR (SEQ ID NO:54). 11 arginine residues, VP22 peptide, and an ANTp peptide (RQIKIWFQNRRMKWKK) (SEQ ID NO:55) or positively charged polypeptides or polynucleotides having 8-15 residues, preferably 9-11 residues. Short, non-peptide polymers that are rich in amines or guanidinium groups are also capable of carrying molecules crossing biological membranes. Penetratin and other derivatives of peptides derived from antennapedia (Cheng, et al., Biomaterials, 32(26):6194-203 (2011) can also be used. Results show that penetratin in which additional Args are added, further enhances uptake and endosomal escape, and IKK NBD, which has an antennapedia domain for permeation as well as a domain that blocks activation of NFkB and has been used safely in the lung for other purposes (von Bismarck, et al., Pulmonary Pharmacology & Therapeutics, 25(3):228-35 (2012), Kamei, et al., Journal Of Pharmaceutical Sciences, 102(11):3998-4008 (2013)).

A “fusogenic peptide” is any peptide with membrane destabilizing abilities. In general, fusogenic peptides have the propensity to form an amphiphilic alpha-helical structure when in the presence of a hydrophobic surface such as a membrane. The presence of a fusogenic peptide induces formation of pores in the cell membrane by disruption of the ordered packing of the membrane phospholipids. Some fusogenic peptides act to promote lipid disorder and in this way enhance the chance of merging or fusing of proximally positioned membranes of two membrane enveloped particles of various nature (e.g. cells, enveloped viruses, liposomes). Other fusogenic peptides may simultaneously attach to two membranes, causing merging of the membranes and promoting their fusion into one. Examples of fusogenic peptides include a fusion peptide from a viral envelope protein ectodomain, a membrane-destabilizing peptide of a viral envelope protein membrane-proximal domain from the cytoplasmic tails.

Other fusogenic peptides often also contain an amphiphilic-region. Examples of amphiphilic-region containing peptides include: melittin, magainins, the cytoplasmic tail of HIV1 gp41, microbial and reptilian cytotoxic peptides such as bomolitin 1, pardaxin, mastoparan, crabrolin, cecropin, entamoeba, and staphylococcal .alpha.-toxin; viral fusion peptides from (1) regions at the N terminus of the transmembrane (TM) domains of viral envelope proteins, e.g. HIV-1, SIV, influenza, polio, rhinovirus, and coxsackie virus; (2) regions internal to the TM ectodomain, e.g. semliki forest virus, sindbis virus, rota virus, rubella virus and the fusion peptide from sperm protein PH-30: (3) regions membrane-proximal to the cytoplasmic side of viral envelope proteins e.g. in viruses of avian leukosis (ALV), Feline immunodeficiency (FIV), Rous Sarcoma (RSV), Moloney murine leukemia virus (MoMuLV), and spleen necrosis (SNV).

### 4. Methods of Making

### a. Conjugates

Methods of polymer synthesis are described, for instance, in Braun et al. (2005) Polymer Synthesis: Theory and Practice. New York, NY: Springer. The polymers may be synthesized via step-growth polymerization, chain-growth polymerization, or plasma polymerization.

In some embodiments an amphiphilic polymer is synthesized starting from a hydrophobic polymer terminated with a first reactive coupling group and a hydrophilic polymer terminated with a second reactive coupling group capable of reacting with the first reactive coupling group to form a covalent bond. One of either the first reactive coupling group or the second reactive coupling group can be a primary amine, where the other reactive coupling group can be an amine-reactive linking group such as isothiocyanates, isocyanates, acyl azides, NHS esters, sulfonyl chlorides, aldehydes, glyoxals, epoxides, oxiranes, carbonates, aryl halides, imidoesters, carbodiimides, anhydrides, and fluorophenyl esters. One of either the first reactive coupling group or the second reactive coupling group can be an aldehyde, where the other reactive coupling group can be an aldehyde reactive linking group such as hydrazides, alkoxyamines, and primary amines. One of either the first reactive coupling group or the second reactive coupling group can be a thiol, where the other reactive coupling group can be a sulfhydryl reactive group such as maleimides, haloacetyls, and pyridyl disulfides.

In some embodiments a hydrophobic polymer terminated with an amine or an amine-reactive linking group is coupled to a hydrophilic polymer terminated with complimentary reactive linking group. For example, an NHS ester activated PLGA can be formed by reacting PLGA-CO(OH) with NHS and a coupling reagent such as dicyclohexylcarbodiimide (DCC) or ethyl(dimethylaminopropyl) carbodiimide (EDC). The NHS ester activated PLGA can be reacted with a hydrophilic polymer terminated with a primary amine, such as a PEG-NH2 to form an amphiphilic PLGA-b-PEG block copolymer.

In some embodiments a conjugate of an amphiphilic polymer with a targeting moiety is formed using the same or similar coupling reactions. In some embodiments the conjugate is made starting from a hydrophilic polymer terminated on one end with a first reactive coupling group and terminated on a second end with a protective group. The hydrophilic polymer is reacted with a targeting moiety having a reactive group that is complimentary to the first reactive group to form a covalent bond between the hydrophilic polymer and the targeting moiety. The protective group can then be removed to provide a second reactive coupling group, for example to allow coupling of a hydrophobic polymer block to the conjugate of the hydrophilic polymer with the targeting moiety. A hydrophobic polymer terminated with a reactive coupling group complimentary to the second reactive coupling group can then be covalently coupled to form the conjugate. Of course, the steps could also be performed in reverse order, i.e. a conjugate of a hydrophobic polymer and a hydrophilic polymer could be formed first followed by deprotection and coupling of the targeting moiety to the hydrophilic polymer block.

In some embodiments a conjugate is formed having a moiety conjugated to both ends of the amphiphilic polymer. For example, an amphiphilic polymer having a hydrophobic polymer block and a hydrophilic polymer block may have targeting moiety conjugated to the hydrophilic polymer block and an additional moiety conjugated to the hydrophobic polymer block. In some embodiments the additional moiety can be a detectable label. In some embodiments the additional moiety is a therapeutic, prophylactic, or diagnostic agent. For example, the additional moiety could be a moiety used for radiotherapy. The conjugate can be prepared starting from a hydrophobic polymer having on one end a first reactive coupling group and another end first protective group and a hydrophilic polymer having on one end a second reactive coupling group and on another end a second protective group. The hydrophobic polymer can be reacted with the additional moiety having a reactive coupling group complimentary to the first reactive coupling group, thereby forming a conjugate of the hydrophobic polymer to the additional moiety. The hydrophilic polymer can be reacted with a targeting moiety having a reactive coupling group complimentary to the second reactive coupling group, thereby forming a conjugate of the hydrophilic polymer to the targeting moiety. The first protective group and the second protective group can be removed to yield a pair of complimentary reactive coupling groups that can be reacted to covalently link the hydrophobic polymer block to the hydrophilic polymer block.

### b. Nanocarrier Formation

### i. Emulsion Methods

In some embodiments, a nanoparticle is prepared using an emulsion solvent evaporation method. For example, a polymeric material is dissolved in a water immiscible organic solvent and mixed with a drug solution or a combination of drug solutions. In some embodiments a solution of a therapeutic, prophylactic, or diagnostic agent to be encapsulated is mixed with the polymer solution. The polymer can be, but is not limited to, one or more of the following: PLA, PGA, PCL, their copolymers, polyacrylates, the aforementioned PEGylated polymers, the aforementioned Polymer-drug conjugates, the aforementioned polymer-peptide conjugates, or the aforementioned fluorescently labeled polymers, or various forms of their combinations. The drug molecules can be, but are not limited to, one or a more of the following: PPARgamma activators (e.g. Rosiglitazone, (RS)-5-[4-(2-[methyl(pyridin-2-yl)amino]ethoxy)benzyl]thiazolidine-2,4-dione, Pioglitazone, (RS)-5-(4-[2-(5-ethylpyridin-2-yl)ethoxy]benzyl)thiazolidine-2,4-dione, Troglitazone, (RS)-5-(4-[(6-hydroxy-2,5,7,8-tetramethylchroman-2-yl)methoxy]benzyl)thiazolidine-2,4-dione etc.), prostagladin E2 analog (PGE2, (5Z,11a,13E,15S)-7-[3-hydroxy-2-(3-hydroxyoct-1-enyl)-5-oxo-cyclopentyl]hept-5-enoic acid etc.), beta3 adrenoceptor agonist (CL 316243, Disodium 5-[(2R)-2-[[(2R)-2-(3-Chlorophenyl)-2-hydroxyethyl]amino]propyl]-1,3-benzodioxole-2,2-dicarboxylate hydrate, etc.), Fibroblast Growth Factor 21 (FGF-21), Irisin, RNA, DNA, chemotherapeutic compounds, nuclear magnetic resonance (NMR) contrast agents, or combinations thereof. The water immiscible organic solvent, can be, but is not limited to, one or more of the following: chloroform, dichloromethane, and acyl acetate. The drug can be dissolved in, but is not limited to, one or more of the following: acetone, ethanol, methanol, isopropyl alcohol, acetonitrile and Dimethyl sulfoxide (DMSO).

In some embodiments the polymer solution contains one or more polymer conjugates as described above. The polymer solution can contain a first amphiphilic polymer conjugate having a hydrophobic polymer block, a hydrophilic polymer block, and a targeting moiety conjugated to the hydrophilic end. In some embodiments the polymer solution contains one or more additional polymers or amphiphilic polymer conjugates. For example the polymer solution may contain, in addition to the first amphiphilic polymer conjugate, one or more hydrophobic polymers, hydrophilic polymers, lipids, amphiphilic polymers, polymer-drug conjugates, or conjugates containing other targeting moieties. By controlling the ratio of the first amphiphilic polymer to the additional polymers or amphiphilic polymer conjugates, the density of the targeting moieties can be controlled. The first amphiphilic polymer may be present from 1% to 100% by weight of the polymers in the polymer solution. For example, the first amphiphilic polymer can be present at 10%, 20%, 30%, 40%, 50%, or 60% by weight of the polymers in the polymer solution.

An aqueous solution is then added into the resulting mixture solution to yield emulsion solution by emulsification. The emulsification technique can be, but not limited to, probe sonication or homogenization through a homogenizer. The plaque-targeted peptides or fluorophores or drugs may be associated with the surface of, encapsulated within, surrounded by, and/or distributed throughout the polymeric matrix of this inventive particle.

### ii. Nanoprecipitation Method

In another embodiment, a multimodal nanoparticle is prepared using nanoprecipitation methods or microfluidic devices. A polymeric material is mixed with a drug or drug combinations in a water miscible organic solvent. The polymer can be, but is not limited to, one or more of the following: PLA, PGA, PCL, their copolymers, polyacrylates, the aforementioned PEGylated polymers, the aforementioned Polymer-drug conjugates, the aforementioned polymer-peptide conjugates, or the aforementioned fluorescently labeled polymers, or various forms of their combinations. The drug molecules can be, but are not limited to, one or more of the following: PPARgamma activators (e.g. Rosiglitazone, (RS)-5-[4-(2-[methyl(pyridin-2-yl)amino]ethoxy)benzyl]thiazolidine-2,4-dione, Pioglitazone, (RS)-5-(4-[2-(5-ethylpyridin-2-yl)ethoxy]benzyl)thiazolidine-2,4-dione, Troglitazone, (RS)-5-(4-[(6-hydroxy-2,5,7,8-tetramethylchroman-2-yl)methoxy]benzyl)thiazolidine-2,4-dione etc.), prostagladin E2 analog (PGE2, (5Z,11a,13E,15S)-7-[3-hydroxy-2-(3-hydroxyoct-1-enyl)-5-oxo-cyclopentyl]hept-5-enoic acid etc.), beta3 adrenoceptor agonist (CL 316243, Disodium 5-[(2R)-2-[[(2R)-2-(3-Chlorophenyl)-2-hydroxyethyl]amino]propyl]-1,3-benzodioxole-2,2-dicarboxylate hydrate, etc.), RNA, DNA, chemotherapeutic compounds, nuclear magnetic resonance (NMR) contrast agents, or combinations thereof. The water miscible organic solvent, can be, but is not limited to, one or more of the following: acetone, ethanol, methanol, isopropyl alcohol, acetonitrile and Dimethyl sulfoxide (DMSO). The resulting mixture solution is then added to a polymer non-solvent, such as an aqueous solution, to yield nanoparticle solution. The plaque-targeted peptides or fluorophores or drugs may be associated with the surface of, encapsulated within, surrounded by, and/or distributed throughout the polymeric matrix of this inventive particle.

### iii. Microfluidics

Methods of making nanoparticles using microfluidics are known in the art. Suitable methods include those described in U.S. Patent Application Publication No. 2010/0022680 A1 by Kamik et al. In general, the microfluidic device comprises at least two channels that converge into a mixing apparatus. The channels are typically formed by lithography, etching, embossing, or molding of a polymeric surface. A source of fluid is attached to each channel, and the application of pressure to the source causes the flow of the fluid in the channel. The pressure may be applied by a syringe, a pump, and/or gravity. The inlet streams of solutions with polymer, targeting moieties, lipids, drug, payload, etc. converge and mix, and the resulting mixture is combined with a polymer non-solvent solution to form the nanoparticles having the desired size and density of moieties on the surface. By varying the pressure and flow rate in the inlet channels and the nature and composition of the fluid sources nanoparticles can be produced having reproducible size and structure.

### iv. Other Methodologies

Solvent Evaporation. In this method the polymer is dissolved in a volatile organic solvent, such as methylene chloride. The drug (either soluble or dispersed as fine particles) is added to the solution, and the mixture is suspended in an aqueous solution that contains a surface active agent such as poly(vinyl alcohol). The resulting emulsion is stirred until most of the organic solvent evaporated, leaving solid microparticles. The resulting microparticles are washed with water and dried overnight in a lyophilizer. Microparticles with different sizes (0.5-1000 microns) and morphologies can be obtained by this method. This method is useful for relatively stable polymers like polyesters and polystyrene.

However, labile polymers, such as polyanhydrides, may degrade during the fabrication process due to the presence of water. For these polymers, the following two methods, which are performed in completely anhydrous organic solvents, are more useful.
Hot Melt Microencapsulation. In this method, the polymer is first melted and then mixed with the solid particles. The mixture is suspended in a non-miscible solvent (like silicon oil), and, with continuous stirring, heated to 5° C. above the melting point of the polymer. Once the emulsion is stabilized, it is cooled until the polymer particles solidify. The resulting microparticles are washed by decantation with petroleum ether to give a free-flowing powder. Microparticles with sizes between 0.5 to 1000 microns are obtained with this method. The external surfaces of spheres prepared with this technique are usually smooth and dense. This procedure is used to prepare microparticles made of polyesters and polyanhydrides. However, this method is limited to polymers with molecular weights between 1,000-50,000 daltons.
Solvent Removal. This technique is primarily designed for polyanhydrides. In this method, the drug is dispersed or dissolved in a solution of the selected polymer in a volatile organic solvent like methylene chloride. This mixture is suspended by stirring in an organic oil (such as silicon oil) to form an emulsion. Unlike solvent evaporation, this method can be used to make microparticles from polymers with high melting points and different molecular weights. Microparticles that range between 1-300 microns can be obtained by this procedure. The external morphology of spheres produced with this technique is highly dependent on the type of polymer used.
Spray-Drying In this method, the polymer is dissolved in organic solvent. A known amount of the active drug is suspended (insoluble drugs) or co-dissolved (soluble drugs) in the polymer solution. The solution or the dispersion is then spray-dried. Typical process parameters for a mini-spray drier (Buchi) are as follows: polymer concentration=0.04 g/mL, inlet temperature=−24° C., outlet temperature=13-15° C., aspirator setting=15, pump setting=10 mL/minute, spray flow=600 Nl/hr, and nozzle diameter=0.5 mm. Microparticles ranging between 1-10 microns are obtained with a morphology which depends on the type of polymer used.
Hydrogel Microparticles. Microparticles made of gel-type polymers, such as alginate, are produced through traditional ionic gelation techniques. The polymers are first dissolved in an aqueous solution, mixed with barium sulfate or some bioactive agent, and then extruded through a microdroplet forming device, which in some instances employs a flow of nitrogen gas to break off the droplet. A slowly stirred (approximately 100-170 RPM) ionic hardening bath is positioned below the extruding device to catch the forming microdroplets. The microparticles are left to incubate in the bath for twenty to thirty minutes in order to allow sufficient time for gelation to occur. Microparticle particle size is controlled by using various size extruders or varying either the nitrogen gas or polymer solution flow rates. Chitosan microparticles can be prepared by dissolving the polymer in acidic solution and crosslinking it with tripolyphosphate. Carboxymethyl cellulose (CMC) microparticles can be prepared by dissolving the polymer in acid solution and precipitating the microparticle with lead ions. In the case of negatively charged polymers (e.g., alginate, CMC), positively charged ligands (e.g., polylysine, polyethyleneimine) of different molecular weights can be ionically attached.

### v. Liposome and Micelle Formation

Liposomes typically have an aqueous core. The aqueous core can contain water or a mixture of water and alcohol. Suitable alcohols include, but are not limited to, methanol, ethanol, propanol, (such as isopropanol), butanol (such as n-butanol, isobutene, sec-butanol, tart-butanol, pentane (such as amyl alcohol, isobutyl carbinol), hexanol (such as 1-hexanol, 2-hexanol, 3-hexanol), heptanol (such as 1-heptanol, 2-heptanol, 3-heptanol and 4-heptanol) or octanol (such as 1-octanol) or a combination thereof.

Liposomes include, for example, small unilamellar vesicles (SUVs) formed by a single lipid bilayer, large unilamellar vesicles (LANs), which are vesicles with relatively large particles formed by a single lipid bilayer, and multi-lamellar vesicles (MLVs), which are formed by multiple membrane layers. Thus, the liposomes can have either one or several aqueous compartments delineated by either one (unilamellar) or several (multilamellar) phospholipid bilayers (Sapra, et al., Curr. Drug Deliv., 2, 369-81 (2005)). Multilamellar liposomes have more lipid bilayers for hydrophobic therapeutic agents to associate with. Thus, potentially greater amounts of therapeutic agent are available within the liposome to reach the target cell.

Liposomes can be of any particle size, for example the mean particle diameter can be about 10 to about 2000 nm. In one embodiment of the invention, the mean particle diameter is about 10, 20, 25, 30, 40, 50, 100, 200, 300, 400, 500, 600, 700, 800, 900, 1,000, 1,250, 1,500, 1,750, 2,000 nm (or any range between about 10 and about 2,000 nm) or more. In one embodiment of the invention, the mean particle diameter is about 2,000, 1,750, 1,500, 1,250, 1,000, 900, 800, 700, 600, 500, 400, 300, 200, 100, 50, 40, 30, 25, 20, 10 rim (or any range between about 2,000 and 10 nm) or less. The mean particle diameter may be about 20 to about 1,000 nm, about 100 to about 1,500 nm, about 100 to about 1,000 nm, about 100 to about 700 nm, about 200 to about 2,000 nm, about 1,000 to about 2,000 nm, or about 750 to about 1,500 nm. Particle diameter refers to the diameter of a particle measured by dynamic light scattering.

The liposomal formulations can contain large liposomes ranging from 1 to 100% of the liposome population in the formulation. In some embodiments, large liposomes represent greater than approximately 50% of the liposome population in the formulation.

Methods of manufacturing liposomes are known in the art and can include, for example, drying down of the lipids from organic solvents, dispersion of the lipids in aqueous media, purification of the resultant liposomes, and analysis of the final product. Some methods of liposome manufacture include, for example, extrusion methods, the Mozafari method, the polyol dilution method, the bubble method, and the heating method.

The micelles may be prepared in a conventional manner, for example, by reversed-phase evaporation, ether injection, surfactant-based techniques, etc. Polymer micelle formulations utilizing a block copolymer having a hydrophilic segment and a hydrophobic segment have been disclosed, e.g., in U.S. Application No. 2016/0114058, WO 2009/142326 A1 and WO 2010/013836 A1.

### c. Methods of Encapsulating or Attaching Molecules to the Surface of the Particles

There are two principle groups of molecules to be encapsulated or attached to the polymer, either directly or via a coupling molecule: targeting molecules, attachment molecules and therapeutic, nutritional, diagnostic or prophylactic agents. These can be coupled using standard techniques. The targeting molecule or therapeutic molecule to be delivered can be coupled directly to the polymer or to a material such as a fatty acid which is incorporated into the polymer.

Functionality refers to conjugation of a ligand to the surface of the particle via a functional chemical group (carboxylic acids, aldehydes, amines, sulfhydryls and hydroxyls) present on the surface of the particle and present on the ligand to be attached. Functionality may be introduced into the particles in two ways.

The first is during the preparation of the particles, for example during the emulsion preparation of particles by incorporation of stablizers with functional chemical groups.

A second is post-particle preparation, by direct crosslinking particles and ligands with homo- or heterobifunctional crosslinkers. This second procedure may use a suitable chemistry and a class of crosslinkers (CDI, EDAC, glutaraldehydes, etc. as discussed in more detail below) or any other crosslinker that couples ligands to the particle surface via chemical modification of the particle surface after preparation. This second class also includes a process whereby amphiphilic molecules such as fatty acids, lipids or functional stabilizers may be passively adsorbed and adhered to the particle surface, thereby introducing functional end groups for tethering to ligands.

### d. Methods of Linking ApoL1 to Targeting Moiety

As discussed above, in some embodiments, the ApoL1, or a fragment, variant, or fusion protein thereof is linked to the targeting moiety such as an antibody and used to deliver the ApoL1 without a nanocarrier delivery system. In addition to conjugating the targeting moiety to the biologically active molecule, the latter can be attached to or associated with the ApoL1, or a fragment, variant, or fusion protein thereof by any method known in the art. For example a ApoL1 and targeting moiety can be expressed together in a host cell as a fusion protein.

An antibody, or active fragment thereof, can be chemically linked to a polypeptide by a peptide bond or by a chemical or peptide linker molecule of the type well known in the art. Methods for attaching a drug or other small molecule pharmaceutical to an antibody fragment are well known and can include used of bifunctional chemical linkers such as N-succinimidyl (4-iodoacetyl)-aminobenzoate; sulfosuccinimidyl(4-iodoacetyl)-aminobenzoate; 4-succinimidyl-oxycarbonyl-.A-inverted.-(2-pyridyldithio) toluene; sulfosuccinimidyl-6-[.alpha.-methyl-.A-inverted.-(pyridyldithiol)-toluamido]hexanoate; N-succinimidyl-3-(−2-pyridyldithio)-proprionate; succinimidyl-6-[3 (-(−2-pyridyldithio)-proprionamido]hexanoate; sulfosuccinimidyl-6-[3 (-(−2-pyridyldithio)-propionamido]hexanoate; 3-(2-pyridyldithio)-propionyl hydrazide, Ellman's reagent, dichlorotriazinic acid, S-(2-thiopyridyl)-L-cysteine, and the like. Further bifunctional linking molecules are discussed in, for example, U.S. Pat. Nos. 5,349,066, 5,618,528, 4,569,789, 4,952,394, and 5,137,877.

The linker can cleavable or noncleavable. Highly stable linkers can reduce the amount of payload that falls off in circulation, thus improving the safety profile, and ensuring that more of the payload arrives at the target cell. Linkers can be based on chemical motifs including disulfides, hydrazones or peptides (cleavable), or thioethers (noncleavable) and control the distribution and delivery of the active agent to the target cell. Cleavable and noncleavable types of linkers have been proven to be safe in preclinical and clinical trials (see, e.g., Brentuximab vedotin which includes an enzyme-sensitive linker cleavable by cathepsin; and Trastuzumab emtansine, which includes a stable, non-cleavable linker). In particular embodiments, the linker is a peptide linker cleavable by Edman degredation (Bqchor, et al., Molecular diversity, 17 (3): 605-11 (2013)).

A non-cleavable linker can keep the active agent within the cell or the target microenvironment. As a result, the entire antibody, linker and active agent enter the targeted cell where the antibody is degraded to the level of an amino acid. The resulting complex between the amino acid of the antibody, the linker and the active agent becomes the active drug. In contrast, cleavable linkers are catalyzed by enzymes in the target cell or microenvironment where it releases the active agent. Once cleaved, the payload can escape from the targeted cell and attack neighboring cells (also referred to as “bystander killing”).

In some embodiments, there is one or more additional molecules, between the active agent and the cleavage site. Other considerations include site-specific conjugation (TDCs) (Axup, Proceedings of the National Academy of Sciences, 109 (40): 16101-6 (2012) and conjugation techniques such as those described in Lyon, et al., Bioconjugate Chem., 32 (10): 1059-1062 (2014), and Kolodych, et al., Bioconjugate Chem., 26 (2): 197-200 (2015) which can improve stability and therapeutic index, and α emitting immunoconjugates (Wulbrand, et al., Multhoff, Gabriele, ed., PLoS ONE. 8 (5): e64730 (2013)).

### III. Pharmaceutical Compositions

The compositions can be formulated with appropriate pharmaceutically acceptable carriers into pharmaceutical compositions for administration to an individual in need thereof. The formulations can be administered enterally (e.g., oral) or parenterally (e.g., by injection or infusion).

The compositions can be formulated for parenteral administration. “Parenteral administration”, as used herein, means administration by any method other than through the digestive tract or non-invasive topical or regional routes. For example, parenteral administration may include administration to a patient intravenously, intradermally, intraarterially, intraperitoneally, intralesionally, intracranially, intraarticularly, intraprostatically, intrapleurally, intratracheally, intravitreally, intratumorally, intramuscularly, subcutaneously, subconjunctivally, intravesicularly, intrapericardially, intraumbilically, or transmucosal (nasal, vaginal, pulmonary, or rectal), e.g., by injection, and by infusion.

In some embodiments, the compositions are administered systemically by, for example, injection or infusion. In some embodiments, the compositions are administered locally by injection or infusion.

Parenteral formulations can be prepared as aqueous compositions using techniques known in the art. Typically, such compositions can be prepared as injectable formulations, for example, solutions or suspensions; solid forms suitable for using to prepare solutions or suspensions upon the addition of a reconstitution medium prior to injection; emulsions, such as water-in-oil (w/o) emulsions, oil-in-water (o/w) emulsions, and microemulsions thereof, liposomes, or emulsomes.

The carrier can be a solvent or dispersion medium containing, for example, water, ethanol, one or more polyols (e.g., glycerol, propylene glycol, and liquid polyethylene glycol), oils, such as vegetable oils (e.g., peanut oil, corn oil, sesame oil, etc.), and combinations thereof. The proper fluidity can be maintained, for example, by the use of a coating, such as lecithin, by the maintenance of the required nanocarrier size in the case of dispersion and/or by the use of surfactants. In many cases, isotonic agents, for example, sugars or sodium chloride, are included.

Solutions and dispersions of the active compounds as the free acid or base or pharmacologically acceptable salts thereof can be prepared in water or another solvent or dispersing medium suitably mixed with one or more pharmaceutically acceptable excipients including, but not limited to, surfactants, dispersants, emulsifiers, pH modifying agents, viscosity modifying agents, and combination thereof.

Suitable surfactants may be anionic, cationic, amphoteric or nonionic surface active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl-beta-alanine, sodium N-lauryl-β-iminodipropionate, myristoamphoacetate, lauryl betaine and lauryl sulfobetaine.

The formulation can contain a preservative to prevent the growth of microorganisms. Suitable preservatives include, but are not limited to, parabens, chlorobutanol, phenol, sorbic acid, and thimerosal. The formulation may also contain an antioxidant to prevent degradation of the active agent(s).

The formulation is typically buffered to a pH of 3-8 for parenteral administration upon reconstitution. Suitable buffers include, but are not limited to, phosphate buffers, acetate buffers, and citrate buffers.

Water soluble polymers are often used in formulations for parenteral administration. Suitable water-soluble polymers include, but are not limited to, polyvinylpyrrolidone, dextran, carboxymethylcellulose, and polyethylene glycol.

Sterile injectable solutions can be prepared by incorporating the active compounds in the required amount in the appropriate solvent or dispersion medium with one or more of the excipients listed above, as required, followed by filtered sterilization. Generally, dispersions are prepared by incorporating the various sterilized active ingredients into a sterile vehicle which contains the basic dispersion medium and the required other ingredients from those listed above. In the case of sterile powders for the preparation of sterile injectable solutions, exemplary methods of preparation include vacuum-drying and freeze-drying techniques which yield a powder of the active ingredient plus any additional desired ingredient from a previously sterile-filtered solution thereof.

Enteral formulations are prepared using pharmaceutically acceptable carriers. As generally used herein “carrier” includes, but is not limited to, diluents, preservatives, binders, lubricants, disintegrators, swelling agents, fillers, stabilizers, and combinations thereof. Polymers used in the dosage form include hydrophobic or hydrophilic polymers and pH dependent or independent polymers. Hydrophobic and hydrophilic polymers include, but are not limited to, hydroxypropyl methylcellulose, hydroxypropyl cellulose, hydroxyethyl cellulose, carboxy methylcellulose, polyethylene glycol, ethylcellulose, microcrystalline cellulose, polyvinyl pyrrolidone, polyvinyl alcohol, polyvinyl acetate, and ion exchange resins.

Carrier also includes all components of the coating composition, which may include plasticizers, pigments, colorants, stabilizing agents, and glidants. Formulations can be prepared using one or more pharmaceutically acceptable excipients, including diluents, preservatives, binders, lubricants, disintegrators, swelling agents, fillers, stabilizers, and combinations thereof.

Controlled release dosage formulations can be prepared as described in standard references such as “Pharmaceutical dosage form tablets”, eds. Liberman et. al. (New York, Marcel Dekker, Inc., 1989), “Remington—The science and practice of pharmacy”, 20th ed., Lippincott Williams & Wilkins, Baltimore, MD, 2000, and “Pharmaceutical dosage forms and drug delivery systems”, 6th Edition, Ansel et al., (Media, PA: Williams and Wilkins, 1995). These references provide information on excipients, materials, equipment and process for preparing tablets and capsules and delayed release dosage forms of tablets, capsules, and granules. These references provide information on carriers, materials, equipment and process for preparing tablets and capsules and delayed release dosage forms of tablets, capsules, and granules.

Stabilizers are used to inhibit or retard drug decomposition reactions which include, by way of example, oxidative reactions. Suitable stabilizers include, but are not limited to, antioxidants, butylated hydroxytoluene (BHT); ascorbic acid, its salts and esters; Vitamin E, tocopherol and its salts; sulfites such as sodium metabisulphite; cysteine and its derivatives; citric acid; propyl gallate, and butylated hydroxyanisole (BHA).

In some embodiments, the compositions are formulated for mucosal administration, such as through nasal, pulmonary, or buccal delivery.

Mucosal formulations may include one or more agents for enhancing delivery through the nasal mucosa. Agents for enhancing mucosal delivery are known in the art, see for example U.S. Patent Application No. 20090252672 to Eddington, and U.S. Patent Application No. 20090047234 to Touitou. Acceptable agents include, but are not limited to, chelators of calcium (EDTA), inhibitors of nasal enzymes (boro-leucin, aprotinin), inhibitors of muco-ciliar clearance (preservatives), solubilizers of nasal membrane (cyclodextrin, fatty acids, surfactants) and formation of micelles (surfactants such as bile acids, Laureth 9 and taurodehydrofusidate (STDHF)). Compositions may include one or more absorption enhancers, including surfactants, fatty acids, and chitosan derivatives, which can enhance delivery by modulation of the tight junctions (TJ) (B. J. Aungst, et al., J. Pharm. Sci. 89(4):429-442 (2000)). In general, the optimal absorption enhancer should possess the following qualities: its effect should be reversible, it should provide a rapid permeation enhancing effect on the cellular membrane of the mucosa, and it should be non-cytotoxic at the effective concentration level and without deleterious and/or irreversible effects on the cellular membrane or cytoskeleton of the TJ.

As provided are pharmaceutical packs and kits including one or more containers filled with antibody or fusion protein. Additionally, one or more other prophylactic or therapeutic agents useful for the treatment of a disease can also be included in the pharmaceutical pack or kit. One embodiment provides a pharmaceutical pack or kit including one or more containers filled with one or more of the ingredients of the pharmaceutical compositions. Optionally associated with such container(s) can be a notice in the form prescribed by a governmental agency regulating the manufacture, use or sale of pharmaceuticals or biological products, which notice reflects approval by the agency of manufacture, use or sale for human administration.

Also provided are kits that can be used in the below methods. In one embodiment, a kit includes one or more antibodies or fusion proteins. In another embodiment, a kit further includes one or more other prophylactic or therapeutic agents useful for the treatment of cancer, in one or more containers. In certain embodiments, the other prophylactic or therapeutic agent is a chemotherapeutic. In other embodiments, the prophylactic or therapeutic agent is a biological or hormonal therapeutic.

### IV. Methods of Use

### A. Methods of Treatment

The disclosed compositions can be used to increase delivery and internalization of ApoL1 to target cells. As shown in the examples below, increasing the internalization of ApoL1, for example by increasing the internalization of recombinant exogenous ApoL1 or endogenous ApoL1-containing complexes such as TLF, in mammalian cells can lead to cell death, including cancer cells death. Thus, the disclosed compositions can be used to increase the level of ApoL1 in target cells and induce their death.

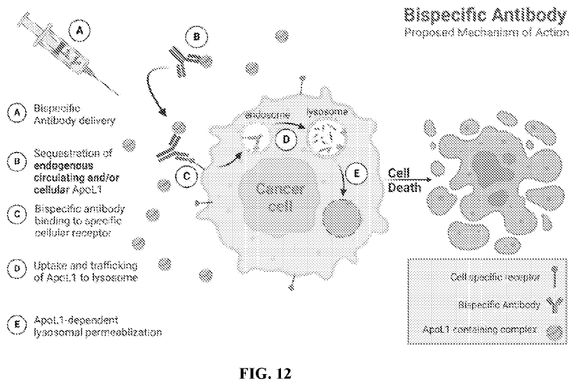

In preferred embodiments, the composition is an ApoL1-containing complex-binding compound is a bi- or multispecific antibody that specifically binds to both the ApoL1-containing complex and a target cell marker. See, e.g., FIG. 12.

Targeting all cell types alone or in combinations are contemplated, including but not limited to, stem cells, skin cells, blood cells, immune cells, muscle cells, nerve cells, cancer cells, virally infected cells, bacterial cells, fungal cells, organ specific cells, and other eukaryotic cells. The cell markers can be specific for endothelial, ectodermal, or mesenchymal cells. Thus, the cells can be mammalian or non-mammalian cells. Most preferably the cells are in a mammalian. The mammalian cells can be human cells. Thus contemplated is targeting of mammalian (e.g., human) and non-mammalian cells in subject (e.g., a human). Thus, the target cells can be bacterial or fungal cells in a mammalian subject such as a human. Additionally, or alternatively, the target cells can be non-mammalian cells infected with another organism such as a virus or bacteria. For example, in some embodiments, the intracellular organism, where the infected cell can be targeted by the presence of extracellular markers. The organism can be bacterial or eukaryotic, such as, Plasmodium falciparum, Toxoplasma gondii, Leishmania sp., Trypanosoma cruzi, Listeria monocytogenes, Chlamydia trachomatis, Coxiella burnetti, Mycobacterium tuberculosis, and other intracellular bacteria and eukaryotes. Extracellular eukaryotes or extracellular stages of intracellular organisms, where the cell can be specifically targeted, include, for example, Toxoplasma gondii, Trichomonas vaginalis, Plasmodium falciparum.

In preferred embodiments, the cancer cells including both blood cancer cells and solid tumor cells, are preferred target cells. The binding activity of the compound can be selected based on the target cells.

Typically the target cells are not Trypanosoma.

### 1. Treatment Methodology

Thus, in some embodiments, the subject is administered an effective amount of a disclosed composition such recombinant ApoL1 and a targeting moiety, or a bi- or multispecific antibody that specifically binds to Hpr or ApoL1 and a target cell antigen.

As used herein, the term “effective amount” or “therapeutically effective amount” means a dosage sufficient to treat, inhibit, or alleviate one or more symptoms of the disorder being treated or to otherwise provide a desired pharmacologic and/or physiologic effect. The precise dosage will vary according to the selected active agent and a variety of factors such as subject-dependent variables (e.g., age, immune system health, etc.), the disease, and the treatment being affected. Typically, the amount is effective to induce or increase ApoL1 mediated cell death of target cells. In some embodiments, the subject has a disease or disorder caused by the target cells, and cell death is induced in an effective amount to treat the disease or disorder. For example, as discussed in more detail below, in some embodiments, the subject has cancer, the target cells are cancer cells, and the treatment increase cell death of the cancer cells.

Any diseased tissue that has a unique biomarker, or biomarker which is overexpressed compared to normal cells, can serve as the target cells, and any such disease that would benefit from increasing cell death of the diseased tissue can be treated.

For example, when the target cells are blood cancer cells, a bispecific antibody that specifically binds to Hpr or ApoL1 and a blood cancer antigen such as BCMA, CD38, CD319/SLAMF-7, TNFRSF17/BCMA, SYND1/CD138, CD229, CD47, CD123/IL3-RA, CD19, CD20, CD22, FcRH5, GPRC5D, CLL-1, PD-L1, or CTLA4 can be used.

Likewise in some embodiments, the targeting moiety of the recombinant ApoL1 composition targets BCMA, CD38, CD319/SLAMF-7, TNFRSF17/BCMA, SYND1/CD138, CD229, CD47, CD123/IL3-RA, CD19, CD20, CD22, FcRH5, GPRC5D, CLL-1, PD-L1, or CTLA4.

Similarly, bi- and other multispecific molecules can target solid tumors or other target cells. Exemplary other antigens, including solid tumor antigens, to which the bi- or other multispecific molecules are provided elsewhere herein.

For example, if the solid tumor is pancreatic cancer, a bispecific antibody that specifically binds to Hpr or ApoL1 and a pancreatic cancer antigen such as Claudin 18.2, MUC, Mesothelin (MSLN), and Myoferlin (MYOF) can be used. Likewise in some embodiments, the targeting moiety of the recombinant ApoL1 composition targets Claudin 18.2, MUC1, Mesothelin (MSLN), and Myoferlin (MYOF).

For example, if the solid tumor is melanoma, a bispecific antibody that specifically binds to Hpr or ApoL1 and a melanoma cancer antigen such as PMEL17 can be used. Likewise in some embodiments, the targeting moiety of the recombinant ApoL1 composition targets PMEL17.

These are non-limiting examples, as many other targets are provided herein and in the art, and as provided herein, the disclosed compositions are methods can be readily modified to target these antigens.

In some embodiments, the composition is administered to a subject in need thereof once every 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days. In some embodiments the composition is administered to a subject once, twice, or three times weekly. In some embodiments, the composition is administered to a subject every other day. In some embodiments, the composition is administered to a subject one, twice, or three times monthly. In some embodiments, the composition is administered for 2, 3, 4, 5, 6, 7, 8, 9, 10, 11, 12, or more weeks or months.

In particular embodiments, for antibodies and other proteins, the dosage administered to a patient is typically 0.0001 mg/kg to 100 mg/kg of the patient's body weight. Preferably, the dosage administered to a patient is between 0.0001 mg/kg and 20 mg/kg, 0.0001 mg/kg and 10 mg/kg, 0.0001 mg/kg and 5 mg/kg, 0.0001 and 2 mg/kg, 0.0001 and 1 mg/kg, 0.0001 mg/kg and 0.75 mg/kg, 0.0001 mg/kg and 0.5 mg/kg, 0.0001 mg/kg to 0.25 mg/kg, 0.0001 to 0.15 mg/kg, 0.0001 to 0.10 mg/kg, 0.001 to 0.5 mg/kg, 0.01 to 0.25 mg/kg or 0.01 to 0.10 mg/kg of the patient's body weight. Generally, human antibodies have a longer half-life within the human body than antibodies from other species due to the immune response to the foreign polypeptides. Thus, lower dosages of human antibodies and less frequent administration is often possible. Further, the dosage and frequency of administration of antibodies or fragments thereof, or fusion proteins may be reduced by enhancing uptake and tissue penetration of the antibodies or fusion proteins by modifications such as, for example, lipidation.

### 2. Diseases to be Treated

### a. Cancer

The disclosed compositions and methods can be used to treat cancer in a subject in need thereof. In a mature animal, a balance usually is maintained between cell renewal and cell death in most organs and tissues. The various types of mature cells in the body have a given life span; as these cells die, new cells are generated by the proliferation and differentiation of various types of stem cells. Under normal circumstances, the production of new cells is so regulated that the numbers of any particular type of cell remain constant. Occasionally, though, cells arise that are no longer responsive to normal growth-control mechanisms. These cells give rise to clones of cells that can expand to a considerable size, producing a tumor or neoplasm. A tumor that is not capable of indefinite growth and does not invade the healthy surrounding tissue extensively is benign. A tumor that continues to grow and becomes progressively invasive is malignant. The term cancer refers specifically to a malignant tumor. In addition to uncontrolled growth, malignant tumors exhibit metastasis. In this process, small clusters of cancerous cells dislodge from a tumor, invade the blood or lymphatic vessels, and are carried to other tissues, where they continue to proliferate. In this way a primary tumor at one site can give rise to a secondary tumor at another site.

The compositions and methods described herein are useful for treating subjects having benign or malignant tumors by delaying or inhibiting the growth of a tumor in a subject, reducing the growth or size of the tumor, inhibiting or reducing metastasis of the tumor, and/or inhibiting or reducing symptoms associated with tumor development or growth.

Malignant tumors that may be treated are classified herein according to the embryonic origin of the tissue from which the tumor is derived. The leukemias and lymphomas are malignant tumors of hematopoietic cells of the bone marrow. Leukemias proliferate as single cells, whereas lymphomas tend to grow as tumor masses. Malignant tumors may show up at numerous organs or tissues of the body to establish a cancer. Carcinomas are tumors arising from endodermal or ectodermal tissues such as skin or the epithelial lining of internal organs and glands. Sarcomas, which arise less frequently, are derived from mesodermal connective tissues such as bone, fat, and cartilage. Malignant tumors may show up at numerous organs or tissues of the body to establish a cancer.

In preferred embodiments, the compositions are used to treat a liquid tumor or blood cancer or tumor of the vascular system, such as multiple myeloma, leukemia (e.g., chronic lymphocytic leukemia, acute myeloid leukemia, acute lymphoblastic leukemia), non-Hodgkin lymphoma, Hodgkin lymphoma, myelodysplastic syndromes (MDS), myeloproliferative neoplasms (MPNs) (or a subcategory thereof, e.g., essential thrombocythemia (ET), myelofibrosis (MF) and polycythemia vera (PV), amyloidosis, Waldenstrom macroglobulinemia, or aplastic anemia.

Additional types of cancer that can be treated with the provided compositions and methods include, but are not limited to, adenocarcinomas and sarcomas of bone, bladder, brain, breast, cervical, colo-rectal, esophageal, kidney, liver, lung, nasopharangeal, pancreatic, prostate, skin, stomach, and uterine. In some embodiments, the disclosed compositions are used to treat multiple cancer types concurrently. The compositions can also be used to treat metastases or tumors at multiple locations.

The disclosed compositions can be used to treat cells undergoing unregulated growth, invasion, or metastasis.

A representative but non-limiting list of cancers that can be treating using the disclosed compositions include cancers of the blood and lymphatic system (including leukemias, Hodgkin's lymphomas, non-Hodgkin's lymphomas, solitary plasmacytoma, multiple myeloma), cancers of the genitourinary system (including prostate cancer, bladder cancer, renal cancer, urethral cancer, penile cancer, testicular cancer), cancers of the nervous system (including mengiomas, gliomas, glioblastomas, ependymomas) cancers of the head and neck (including squamous cell carcinomas of the oral cavity, nasal cavity, nasopharyngeal cavity, oropharyngeal cavity, larynx, and paranasal sinuses), lung cancers (including small cell and non-small cell lung cancer), gynecologic cancers (including cervical cancer, endometrial cancer, vaginal cancer, vulvar cancer ovarian and fallopian tube cancer), gastrointestinal cancers (including gastric, small bowel, colorectal, liver, hepatobiliary, and pancreatic cancers), skin cancers (including melanoma, squamous cell carcinomas, and basal cell carcinomas), breast cancer (including ductal and lobular cancer and triple negative breast cancers), and pediatric cancers (including neuroblastoma, Ewing's sarcoma, Wilms tumor, medulloblastoma).

In some embodiments, the tumor is a solid tumor characterized by increased vascular permeability, and optionally the enhanced permeability and retention (EPR) effect increases localization of the ApoL1-containing complex-antibody complex to the tumor site relative to less vascularized tumors.

### b. Other Diseases

Disturbances in cell death pathways at the molecular level can be linked to the pathogenesis not only of cancer, but also other diseases of enormous social importance, such as infections such as viral infections (e.g., HIV) bacterial infections, fungal infections, non-mammalian eukaryotic cell infections, etc., atherosclerosis, ischemia, reperfusion injury, infection, inflammation, autoimmune, and neurological disorders (Kaminskyy and Zhivotovsky, Cell Death & Disease, volume 9, Article number: 110 (2018). Thus, the disclosed compositions and methods can be used to treat such diseases.

For example, neutrophils are involved in various types of tissue inflammation and disease, and targeting them for cell death according to the disclosed compositions and methods may be used treat autoimmune and inflammatory diseases.

In some embodiments, the compositions and methods can be used to treat infections. For example, in some embodiments, foreign cells such as bacteria or fungi that are specifically directly targeted for cell death. In other embodiments, infections are treated by targeting infected mammalian (e.g., host) cells, e.g., by targeting extracellular markers on the infected mammalian cells. Exemplary foreign and infected target cells are discussed above.

For example, one strategy to eliminate the latent HIV-1 reservoir is the shock and kill approach that involves the use of latency-reversing agents (LRAs) to reactivate viral gene expression (Shock), followed by the elimination of cells harboring reactivated provirus (Kill) (Rao, et al., Nat Commun. 12(1):2475 (2021) doi:10.1038/s41467-021-22608-z). Thus, in some embodiments, HIV-infected cells such as CD4+ T cells, are targeted for cell death according to the disclosed compositions and methods, and thus treat HIV.

### 3. Combination Therapy

The disclosed compositions can be used in combination with one or more additional active agents, which can be administered in the same or different admixture. Exemplary additional active agents include standard chemotherapy, radiation therapy, and other anti-cancer treatments. In some embodiments, the additional active agent is a therapeutic drug. The majority of chemotherapeutic drugs can be divided into alkylating agents, antimetabolites, anthracyclines, plant alkaloids, topoisomerase inhibitors, monoclonal antibodies, and other antitumour agents.

Non-limiting examples of antineoplastic drugs that damage DNA or inhibit DNA repair include carboplatin, carmustine, chlorambucil, cisplatin, cyclophosphamide, dacarbazine, daunorubicin, doxorubicin, epirubicin, idarubicin, ifosfamide, lomustine, mechlorethamine, mitoxantrone, oxaliplatin, procarbazine, temozolomide, and valrubicin.

In some embodiments, the antineoplastic drug is a histone deacetylase inhibitor, which suppresses DNA repair at the transcriptional level and disrupt chromatin structure. In some embodiments, the antineoplastic drug is a proteasome inhibitor, which suppresses DNA repair by disruption of ubiquitin metabolism in the cell. Ubiquitin is a signaling molecule that regulates DNA repair. In some embodiments, the antineoplastic drug is a kinase inhibitor, which suppresses DNA repair by altering DNA damage response signaling pathways.

Additional antineoplastic drugs include, but are not limited to, alkylating agents (such as cisplatin, carboplatin, oxaliplatin, mechlorethamine, cyclophosphamide, chlorambucil, dacarbazine, lomustine, carmustine, procarbazine, chlorambucil and ifosfamide), antimetabolites (such as fluorouracil, gemcitabine, methotrexate, cytosine arabinoside, fludarabine, and floxuridine), some antimitotics, and vinca alkaloids such as vincristine, vinblastine, vinorelbine, and vindesine), anthracyclines (including doxorubicin, daunorubicin, valrubicin, idarubicin, and epirubicin, as well as actinomycins such as actinomycin D), cytotoxic antibiotics (including mitomycin, plicamycin, and bleomycin), and topoisomerase inhibitors (including camptothecins such as irinotecan and topotecan and derivatives of epipodophyllotoxins such as amsacrine, etoposide, etoposide phosphate, and teniposide) and cytoskeletal targeting drugs such as paclitaxel.

In some embodiments the active agent is a radiosensitizer. Examples of known radiosensitizers include cisplatin, gemcitabine, 5-fluorouracil, pentoxifylline, vinorelbine, PARP inhibitors, histone deacetylase inhibitors, and proteasome inhibitors.

In some embodiments, the additional active agent is radiation. Radiation therapy (a.k.a. radiotherapy) is the medical use of ionizing radiation as part of cancer treatment to control malignant cells.

### B. Methods of Detection

The disclosed ApoL1-containing complex-binding antibodies, e.g., anti-Hpr and anti-ApoL1 antibodies, and their antigen-binding fragments can be used to, for example, detect ApoL1-containing complexes such as TLF, and components thereof such as Hpr and ApoL1. Thus, the disclosure provides for assaying for presences of ApoL1-containing complexes such as TLF, or components thereof such as Hpr and ApoL1, in cells or in a tissue or other biological sample of a subject using one or more antibodies (or fragments thereof) that immunospecifically bind to such antigens. Such antibodies and fragments are preferably employed in immunoassays, such as Western blotting, enzyme linked immunosorbent assay (ELISA), the radioimmunoassay (RIA), fluorescence-activated cell sorting (FACS), immunohistochemistry (IHC), etc.

In some embodiments, detection methods include, a) administering to a subject (for example, parenterally, subcutaneously, or intraperitoneally) an effective amount of a labeled antibody or antigen-binding fragment that immunospecifically binds to ApoL1-containing complexes such as TLF; b) waiting for a time interval following the administration for permitting the labeled molecule to preferentially concentrate at sites in the subject where ApoL1-containing complexes such as TLF are located (and for unbound labeled molecule to be cleared to background level); c) determining background level; and d) detecting the labeled antibody in the subject, such that detection of labeled antibody above the background level indicates the level and/or location of ApoL1-containing complexes such as TLF in the subject. In accordance with this embodiment, the antibody is labeled with an imaging moiety which is detectable using an imaging system known to one of skill in the art. Background level can be determined by various methods including, comparing the amount of labeled molecule detected to a standard value previously determined for a particular system.

It will be understood in the art that the size of the subject and the imaging system used will determine the quantity of imaging moiety needed to produce diagnostic images. In vivo tumor imaging is described in S.W. Burchiel et al., “Immunopharmacokinetics of Radiolabeled Antibodies and Their Fragments,” (Chapter 13 in TUMOR IMAGING: THE RADIOCHEMICAL DETECTION OF CANCER, S. W. Burchiel and B. A. Rhodes, eds., Masson Publishing Inc. (1982).

Depending on several variables, including the type of label used and the mode of administration, the time interval following the administration for permitting the labeled molecule to preferentially concentrate at sites in the subject and for unbound labeled molecule to be cleared to background level is 6 to 48 hours or 6 to 24 hours or 6 to 12 hours. In another embodiment the time interval following administration is 5 to 20 days or 5 to 10 days.

Presence of the labeled molecule can be detected in the subject using methods known in the art for in vivo scanning. These methods depend upon the type of label used. Skilled artisans will be able to determine the appropriate method for detecting a particular label. Methods and devices that may be used in the disclosed diagnostic methods include, but are not limited to, computed tomography (CT), whole body scan such as position emission tomography (PET), magnetic resonance imaging (MRI), and sonography. In a specific embodiment, the molecule is labeled with a radioisotope and is detected in the patient using a radiation responsive surgical instrument (Thurston et al., U.S. Pat. No. 5,441,050). In another embodiment, the molecule is labeled with a fluorescent compound and is detected in the patient using a fluorescence responsive scanning instrument. In another embodiment, the molecule is labeled with a positron emitting metal and is detected in the patient using positron emission-tomography. In yet another embodiment, the molecule is labeled with a paramagnetic label and is detected in a patient using magnetic resonance imaging (MRI).

The disclosed invention can be further understood by the following numbered paragraphs:

1. A method of increasing cell death of target cells in a mammalian subject in need thereof including administering the subject an effective amount of a composition that increases Apolipoprotein Li (ApoL1) in the target cells.

2. The method of paragraph 1, wherein the composition increases accumulation of endogenous ApoL1 in the target cells.

3. The method of paragraph 2, wherein the endogenous ApoL1 is a component of an ApoL1-containing complex.

4. The method of paragraph 3, wherein the ApoL1-containing complex is a Trypanosome Lytic Factor (TLF), optionally TLF-1 and/or TLF-2.

5. The method of any one of paragraphs 1-4, wherein the composition includes a first antigen binding fragment that binds to ApoL1 or an ApoL1-containing complex and a targeting moiety that targets the composition to target cells, optionally wherein the composition is a bi- or multispecific antibody a first antigen binding fragment that binds to ApoL1 or an ApoL1-containing complex optionally a TLF and a second antigen binding fragment that binds to a cell specific antigen.

6. The method of paragraph 1, wherein the composition includes a ApoL1 or a function fragment or variant thereof and a targeting moiety for a cell specific antigen.

7. The method of paragraph 6, wherein the composition includes the ApoL1 or a function fragment or variant thereof conjugated or fused directly or indirectly the targeting moiety.

8. The method of paragraphs 6 or 7, wherein the composition includes a delivery vehicle, optionally liposomes or polymeric nanoparticles.

9. The method of paragraph 8, wherein the targeting moiety is conjugated or fused to the delivery vehicle.

10. The method of any one of paragraphs 6-9, wherein the targeting moiety is an antibody or antigen bind fragment.

11. The method of any one of paragraphs 1-10, wherein the cell specific antigen is specific for diseased cells.

12. The method of paragraph 11, wherein the diseased cells are cancer cells.

13. The method of paragraph 12, wherein the cancer cells are blood cancer cells.

14. The method of any one of paragraphs 1-13, where the subject suffers from a disease caused by the target cells.

15. The method of paragraph 14, wherein the composition is administered in an effective amount to treat the disease.

16. The method of any one of paragraphs 1-15, wherein the cell specific antigen is not a trypanosome specific surface antigen.

17. The method of any one of paragraphs 1-16, wherein the subject does not have trypanosomiasis.

18. A composition including ApoL1 or a function fragment or variant thereof and a targeting moiety, wherein the targeting moiety does not target a trypanosome specific surface antigen.

19. The composition of paragraph 18, wherein the ApoL1 or a function fragment or variant thereof conjugated or fused directly or indirectly the targeting moiety.

20. The composition of paragraph 19, wherein the composition includes a delivery vehicle, optionally liposomes or polymeric nanoparticles, optionally, wherein the targeting moiety is conjugated or fused to the delivery vehicle.

21. An antibody or antigen binding fragment including:
the three complementarity determining regions (CDRs) of the heavy chain variable domain of SEQ ID NO:24, or variants or humanized forms thereof with at least 70, 80, 90, or 95% sequence identity thereto, and the three complementarity determining regions (CDRs) of the light chain variable domain of SEQ ID NO:36 or SEQ ID NO:77, or variants or humanized forms thereof with at least 70, 80, 90, or 95% sequence identity thereto,
wherein the antibody or antigen binding fragment binds to Apolipoprotein Li (ApoL1).

22. The antibody or antigen binding fragment of paragraph 21, wherein the heavy and light chain variable domain CDRs include:

(SEQ ID NO: 25)
TYAMS,
(SEQ ID NO: 26)
EISNGGLYTYYPDTVTG,
(SEQ ID NO: 27)
ENRNWYFDL,
(SEQ ID NO: 37)
RSSQSIVNSNGNTYLE,
and
(SEQ ID NO: 38)
KVSNRFS,
(SEQ ID NO: 39)
FQGSHVPLT,
or
variants or humanized forms thereof with at least 70, 80, 90, or 95%
sequence identity thereto;
(SEQ ID NO: 28)
GFTFSTYA,
(SEQ ID NO: 29)
ISNGGLYT,
(SEQ ID NO: 30)
IRENRNWYFDL,
(SEQ ID NO: 40)
QSIVNSNGNTY,
KVS,
and
(SEQ ID NO: 39)
FQGSHVPLT,
or
variants or humanized forms thereof with at least 70, 80, 90, or 95%
sequence identity thereto;
or
(SEQ ID NO: 31)
GFTFSTY,
(SEQ ID NO: 32)
SNGGLY,
(SEQ ID NO: 27)
ENRNWYFDL,
(SEQ ID NO: 37)
RSSQSIVNSNGNTYLE,
(SEQ ID NO: 38)
KVSNRFS,
and
(SEQ ID NO: 39)
FQGSHVPLT,
or
variants or humanized forms thereof with at least 70, 80, 90, or 95%
sequence identity thereto.

23. The antibody or antigen binding fragment of paragraphs 21 or 22, wherein the heavy and light chain variable domain CDRs include:

(SEQ ID NO: 25)
TYAMS,
(SEQ ID NO: 26)
EISNGGLYTYYPDTVTG,
(SEQ ID NO: 27)
ENRNWYFDL,
(SEQ ID NO: 37)
RSSQSIVNSNGNTYLE,
and
(SEQ ID NO: 38)
KVSNRFS,
(SEQ ID NO: 39)
FQGSHVPLT;
(SEQ ID NO: 28)
GFTFSTYA,
(SEQ ID NO: 29)
ISNGGLYT,
(SEQ ID NO: 30)
IRENRNWYFDL,
(SEQ ID NO: 40)
QSIVNSNGNTY,
KVS,
and
(SEQ ID NO: 39)
FQGSHVPLT;
or
(SEQ ID NO: 31)
GFTFSTY,
(SEQ ID NO: 32)
SNGGLY,
(SEQ ID NO: 27)
ENRNWYFDL,
(SEQ ID NO: 37)
RSSQSIVNSNGNTYLE,
(SEQ ID NO: 38)
KVSNRFS,
and
(SEQ ID NO: 39)
FQGSHVPLT.

24. The antibody or antigen binding fragment of any one of paragraphs 21-23 including a heavy chain variable domain including the amino acid sequence of SEQ ID NO:24 or a variant or humanized form thereof with at least 70, 80, 90, or 95% sequence identity thereto and a light chain variable domain including the amino acid sequence of SEQ ID NO:36 or SEQ ID NO:77 or a variant or humanized form thereof with at least 70, 80, 90, or 95% sequence identity thereto.

25. The antibody or antigen binding fragment thereof of any one of paragraphs 21-24 including a heavy chain variable domain including the amino acid sequence of SEQ ID NO:24 and a light chain variable domain including the amino acid sequence of SEQ ID NO:36 or SEQ ID NO:77.

26. An antibody or antigen binding fragment including:
the three complementarity determining regions (CDRs) of the heavy chain variable domain of SEQ ID NO:3, or variants or humanized forms thereof with at least 70, 80, 90, or 95% sequence identity thereto, and the three complementarity determining regions (CDRs) of the light chain variable domain of SEQ ID NO:14, or variants or humanized forms thereof with at least 70, 80, 90, or 95% sequence identity thereto,
wherein the antibody or antigen binding fragment binds to Haptoglobin related protein (Hpr).

27. The antibody or antigen binding fragment of paragraph 26, wherein the heavy and light chain variable domain CDRs include:

(SEQ ID NO: 4)
NYGMN,
(SEQ ID NO: 5)
WINSYTGEATYTDDLKG,
(SEQ ID NO: 6)
EGYGDYGYSFDY,
(SEQ ID NO: 16)
RATKNIYTYLA,
(SEQ ID NO: 17)
NAKTLAE,
and
(SEQ ID NO: 18)
QHHYGTPRT,
or
variants or humanized forms thereof with at least 70, 80, 90, or 95%
sequence identity thereto;
(SEQ ID NO: 7)
GYIFTNYG,
(SEQ ID NO: 8)
INSYTGEA,
(SEQ ID NO: 9)
AREGYGDYGYSFDY,
(SEQ ID NO: 19)
KNIYTY,
NAK,
and
(SEQ ID NO: 18)
QHHYGTPRT,
or
variants or humanized forms thereof
with at least 70, 80, 90, or 95% sequence identity thereto;
or
(SEQ ID NO: 10)
GYIFTNY,
(SEQ ID NO: 11)
NSYTGE,
(SEQ ID NO: 6)
EGYGDYGYSFDY,
(SEQ ID NO: 16)
RATKNIYTYLA,
(SEQ ID NO: 17)
NAKTLAE,
and
(SEQ ID NO: 18)
QHHYGTPRT,
or
variants or humanized forms thereof with at least 70, 80, 90, or 95%
sequence identity thereto.

28. The antibody or antigen binding fragment of paragraphs 26 or 27, wherein the heavy and light chain variable domain CDRs include:

(SEQ ID NO: 4)
NYGMN,
(SEQ ID NO: 5)
WINSYTGEATYTDDLKG,
(SEQ ID NO: 6)
EGYGDYGYSFDY,
(SEQ ID NO: 16)
RATKNIYTYLA,
(SEQ ID NO: 17)
NAKTLAE,
and
(SEQ ID NO: 18)
QHHYGTPRT;
(SEQ ID NO: 7)
GYIFTNYG,
(SEQ ID NO: 8)
INSYTGEA,
(SEQ ID NO: 9)
AREGYGDYGYSFDY,
(SEQ ID NO: 19)
KNIYTY,
NAK,
and
(SEQ ID NO: 18)
QHHYGTPRT;
or
(SEQ ID NO: 10)
GYIFTNY,
(SEQ ID NO: 11)
NSYTGE,
(SEQ ID NO: 6)
EGYGDYGYSFDY,
(SEQ ID NO: 16)
RATKNIYTYLA,
(SEQ ID NO: 17)
NAKTLAE,
and
(SEQ ID NO: 18)
QHHYGTPRT.

29. The antibody or antigen binding fragment of any one of paragraphs 26-28 including a heavy chain variable domain including the amino acid sequence of SEQ ID NO:3 or a variant or humanized form thereof with at least 70, 80, 90, or 95% sequence identity thereto and a light chain variable domain including the amino acid sequence of SEQ ID NO:14 or a variant or humanized form thereof with at least 70, 80, 90, or 95% sequence identity thereto.

30. The antibody or antigen binding fragment thereof of any one of paragraphs 26-29 including a heavy chain variable domain including the amino acid sequence of SEQ ID NO:3 and a light chain variable domain including the amino acid sequence of SEQ ID NO:14.

31. An antibody or antigen binding fragment including:
the three complementarity determining regions (CDRs) of the heavy chain variable domain of SEQ ID NO:56, or variants or humanized forms thereof with at least 70, 80, 90, or 95% sequence identity thereto, and the three complementarity determining regions (CDRs) of the light chain variable domain of SEQ ID NO:65, or variants or humanized forms thereof with at least 70, 80, 90, or 95% sequence identity thereto,
wherein the antibody or antigen binding fragment binds to Haptoglobin related protein (Hpr).

32. The antibody or antigen binding fragment of paragraph 31, wherein the heavy and light chain variable domain CDRs include:

(SEQ ID NO: 57)
DYSIH,
(SEQ ID NO: 58)
WKHTESGESTYADDFKG,
(SEQ ID NO: 59)
GANYGSLLDY,
(SEQ ID NO: 66)
RASKSVSTSGYSYMH,
(SEQ ID NO: 67)
LASNLES,
(SEQ ID NO: 68)
QHNRELPLT,
or
variants or humanized forms thereof with at least 70, 80, 90, or 95%
sequence identity thereto;
(SEQ ID NO: 60)
GFTFTDYS,
(SEQ ID NO: 61)
KHTESGES,
(SEQ ID NO: 62)
ARGANYGSLLDY,
(SEQ ID NO: 69)
KSVSTSGYSY,
LAS,
(SEQ ID NO: 68)
QHNRELPLT,
or
variants or humanized forms
thereof with at least 70, 80, 90, or 95% sequence identity thereto;
or
(SEQ ID NO: 63)
GFTFTDY,
(SEQ ID NO: 64)
HTESGE,
(SEQ ID NO: 59)
GANYGSLLDY,
(SEQ ID NO: 66)
RASKSVSTSGYSYMH,
(SEQ ID NO: 67)
LASNLES,
(SEQ ID NO: 68)
QHNRELPLT,
or
variants or humanized forms thereof with at least 70, 80, 90, or 95%
sequence identity thereto.

33. The antibody or antigen binding fragment of paragraphs 31 or 32, wherein the heavy and light chain variable domain CDRs include:

(SEQ ID NO: 57)
DYSIH,
(SEQ ID NO: 58)
WKHTESGESTYADDFKG,
(SEQ ID NO: 59)
GANYGSLLDY,
(SEQ ID NO: 66)
RASKSVSTSGYSYMH,
(SEQ ID NO: 67)
LASNLES,
(SEQ ID NO: 68)
QHNRELPLT;
(SEQ ID NO: 60)
GFTFTDYS,
(SEQ ID NO: 61)
KHTESGES,
(SEQ ID NO: 62)
ARGANYGSLLDY,
(SEQ ID NO: 69)
KSVSTSGYSY,
LAS,
(SEQ ID NO: 68)
QHNRELPLT;
or
(SEQ ID NO: 63)
GFTFTDY,
(SEQ ID NO: 64)
HTESGE,
(SEQ ID NO: 59)
GANYGSLLDY,
(SEQ ID NO: 66)
RASKSVSTSGYSYMH,
(SEQ ID NO: 67)
LASNLES,
(SEQ ID NO: 68)
QHNRELPLT.

34. The antibody or antigen binding fragment of any one of paragraphs 31-33 including a heavy chain variable domain including the amino acid sequence of SEQ ID NO:56 or a variant or humanized form thereof with at least 70, 80, 90, or 95% sequence identity thereto and a light chain variable domain including the amino acid sequence of SEQ ID NO:65 or a variant or humanized form thereof with at least 70, 80, 90, or 95% sequence identity thereto.

35. The antibody or antigen binding fragment thereof of any one of paragraphs 31-34 including a heavy chain variable domain including the amino acid sequence of SEQ ID NO:56 and a light chain variable domain including the amino acid sequence of SEQ ID NO:65.

36. The antibody or antigen binding fragment of any one of paragraphs 21-35, wherein the antibody or antigen binding fragment binds to an ApoL1-containing complex, optionally, wherein the ApoL1-containing complex is Trypanosome Lytic Factor (TLF).

37. The antibody or antigen binding fragment of paragraph 36, wherein the TLF is endogenous human TLF.

38. The antibody or antigen binding fragment of paragraphs 36 or 37, wherein the antibody or antigen binding fragment can bind to the ApoL1-containing complex under physiological conditions.

39. The antibody or antigen binding fragment of any one of paragraphs 36-38, wherein the antibody or antigen binding fragment can bind to an ApoL1-containing complex in a subject, optionally wherein the subject is a human.

40. The antibody or antigen binding fragment of any one of paragraphs 21-39, wherein the antibody is not a mouse IgG1 or IgG2a.

41. The antibody or antigen binding fragment of any one of paragraphs 21-40 including one or more constant domains from an immunoglobulin constant region (Fc).

42. The antibody or antigen binding fragment of paragraph 41 wherein the constant domains are human constant domains.

43. The antibody or antigen binding fragment of paragraph 42 wherein the human constant domains are IgA, IgD, IgE, IgG or IgM domains.

44. The antibody or antigen binding fragment of paragraph 43 wherein human IgG constant domains are IgG1, IgG2, IgG3, or IgG4 domains.

45. The antibody or antigen binding fragment of any one of paragraphs 21-44 wherein the antibody or antigen binding fragment is detectably labeled or includes a conjugated toxin, drug, receptor, enzyme, receptor ligand.

46. The antibody or antigen binding fragment of any one of paragraph 21-45, wherein the antibody is a monoclonal antibody, a human antibody, a chimeric antibody, or a humanized antibody.

47. The antibody or antigen binding fragment of any one of paragraphs 21-46, wherein the antibody is a bispecific, trispecific or multispecific antibody.

48. The antibody or antigen binding fragment of paragraph 47, wherein the bispecific, trispecific or multispecific antibody includes a second antigen binding fragment that binds to a cell specific antigen.

49. A bispecific, trispecific or multispecific antibody including a first antigen binding fragment that binds to ApoL1 or an ApoL1-containing complex optionally a TLF and a second antigen binding fragment that binds to a cell specific antigen.

50. The antibody or antigen binding fragment of paragraphs 48 or 49, wherein the cell specific antigen is a cancer or tumor antigen.

51. The antibody or antigen binding fragment of paragraph 50, wherein the cancer or tumor antigen is a blood cancer antigen or a solid tumor antigen optionally selected from Claudin 18.2, MUC1, Mesothelin (MSLN), Myoferlin (MYOF), and PMEL17.

52. The antibody or antigen binding fragment of paragraph 51, wherein the blood cancer antigen is selected from the group consisting of BCMA, CD38, CD319/SLAMF-7, TNFRSF17/BCMA, SYND1/CD138, CD229, CD47, CD123/IL3-RA, CD19, CD20, CD22, FcRH5, GPRC5D, CLL-1, PD-L1, and CTLA4.

53. The antibody or antigen binding fragment of any one of paragraphs 48-52, wherein the cell specific antigen is BCMA.

54. The antibody or antigen binding fragment of paragraph 28, wherein the second antigen binding fragment includes the three CDRs of the heavy chain variable domain of SEQ ID NO:41 or variant or humanized form thereof with at least 70, 80, 90, or 95% sequence identity thereto and the three CDRs of the light chain variable domain of SEQ ID NO:42 or variant or humanized form thereof with at least 70, 80, 90, or 95% sequence identity thereto.

55. The antibody or antigen binding fragment of paragraph 54, wherein the second antigen binding fragment includes the six CDRs including the amino acid sequences:
CDR1H: SYAMS (SEQ ID NO:43) or variant or humanized form thereof with at least 70, 80, 90, or 95% sequence identity thereto,
CDR2H: AISGSGGSTYYADSVKG (SEQ ID NO:44) or variant or humanized form thereof with at least 70, 80, 90, or 95% sequence identity thereto,
CDR3H: VAPYFAPFDY (SEQ ID NO:45) or variant or humanized form thereof with at least 70, 80, 90, or 95% sequence identity thereto,
CDR1L: RASQSVSSSYLA (SEQ ID NO:46) or variant or humanized form thereof with at least 70, 80, 90, or 95% sequence identity thereto,
CDR2L: GAS SRAT (SEQ ID NO:47) or variant or humanized form thereof with at least 70, 80, 90, or 95% sequence identity thereto, and
CDR3L: QQYGNPPLYT (SEQ ID NO:48) or variant or humanized form thereof with at least 70, 80, 90, or 95% sequence identity thereto.

56. The antibody or antigen binding fragment of any one of paragraphs 53-55, wherein the second antigen binding fragment includes a heavy chain variable domain including the amino acid sequence of SEQ ID NO:41 or variant or humanized form thereof with at least 70, 80, 90, or 95% sequence identity thereto and a light chain variable domain including the amino acid sequence of SEQ ID NO:42 or variant or humanized form thereof with at least 70, 80, 90, or 95% sequence identity thereto.

57. The antibody or antigen binding fragment of paragraph 56, wherein the second antigen binding fragment includes a heavy chain variable domain including the amino acid sequence of SEQ ID NO:41 and a light chain variable domain including the amino acid sequence of SEQ ID NO:42,
optionally wherein the second antigen binding fragment includes the amino acid sequence of SEQ ID NO:51.

58. The antibody or antigen binding fragment of any one of paragraphs 49-56 including the amino acid sequence of SEQ ID NO:71 or variant or humanized form thereof with at least 70, 80, 90, or 95% sequence identity thereto and/or the amino acid sequence of SEQ ID NO:72 or variant or humanized form thereof with at least 70, 80, 90, or 95% sequence identity thereto.

59. The antibody or antigen binding fragment of any one of paragraphs 49-56 including the amino acid sequence of SEQ ID NO: 71 and SEQ ID NO:72.

60. The antibody or antigen binding fragment of any one of paragraphs 49-56 including two copies each of the amino acid sequences of SEQ ID NO:70 and SEQ ID NO:72

61. An anti-ApoL1, anti-cell specific antigen IgG1-scFv bispecific chimeric antibody.

62. An anti-Hpr, anti-cell specific antigen IgG1-scFv bispecific chimeric antibody.

63. A nucleic acid encoding the antibody or antigen binding fragment of any one of paragraphs 21-63.

64. The nucleic acid of paragraph 63 operably linked to an expression control sequence.

65. An expression vector including the nucleic acid of paragraphs 63 or 64.

66. A cell including the nucleic acid of paragraphs 63 or 64, or the expression vector of paragraph 65, optionally wherein the cell is a mammalian cell.

67. An immunocomplex including the antibody or antigen binding fragment of any one of paragraphs 21-62 bound to ApoL1 or an ApoL1-containing complex, optionally wherein the complex is a TLF.

68. An immunocomplex including the antibody or antigen binding fragment of any one of paragraphs 47-62.

69. A method of inducing cell death including contacting target cells with the immunocomplex of paragraphs 67 or 68.

70. The method of paragraph 69, wherein the contacting occurs in vitro.

71. The method of paragraph 69, wherein the contracting occurs in vivo in a subject.

72. The method of paragraph 71, wherein the subject has cancer.

73. A pharmaceutical composition including the antibody or antigen binding fragment of any one of paragraphs 21-62.

74. A method of treating cancer including administering the subject an effective amount of the antibody or antigen binding fragment of any one of paragraphs 21-62.

75. The method of paragraphs 74, including administering the subject an effective amount of the antibody or antigen binding fragment of any one of paragraphs 22-62.

76. The method of paragraphs 74 or 75, wherein the cancer is blood cancer.

77. The method of paragraph 76, wherein the cancer is multiple myeloma, leukemia (e.g., chronic lymphocytic leukemia, acute myeloid leukemia, acute lymphoblastic leukemia), non-Hodgkin lymphoma, Hodgkin lymphoma, myelodysplastic syndromes (MDS), myeloproliferative neoplasms (MPNs) (or a subcategory thereof, e.g., essential thrombocythemia (ET), myelofibrosis (MF) and polycythemia vera (PV), amyloidosis, Waldenstrom macroglobulinemia, or aplastic anemia.

78. The method of paragraphs 74 or 75, wherein the cancer is a solid cancer.

79. A method of treating a subject in need thereof including administering the subject the composition of any one of paragraphs 1-62.

80. The method of paragraph 79, wherein the subject has cancer.

81. The method of any one of paragraphs 1-5 or 11-17 wherein the composition includes the antibody of any one of paragraphs 21-62.

82. The composition or method of any one of paragraphs 1-81, wherein the target cells are mammalian cells.

83. The composition or method of paragraph 82, wherein the mammalian cells are infected cells.

84. The composition or method of paragraph 83, wherein the infected cells are infected with a virus, bacteria, or eukaryotic intracellular organism, optionally selected from HIV, Plasmodium falciparum, Toxoplasma gondii, Leishmania sp., Trypanosoma cruzi, Listeria monocytogenes, Chlamydia trachomatis, Coxiella bumetti, Mycobacterium tuberculosis, and Trichomonas vaginalis.

85. The composition or method of any one of paragraphs 1-81, wherein the target cells are non-mammalian cells.

86. The composition or method of paragraph 85, wherein the non-mammalian cells are bacteria, fungi, or non-mammalian eukaryotic cells.

87. The composition or method of paragraph 86, wherein the non-mammalian cells are not Trypanosoma.

88. The composition or method of any one of the foregoing paragraphs wherein the subject is a mammal, optionally a human.

### EXAMPLES

### Example 1: TLF does not Bind Mammalian Cells with High Affinity

### Materials and Methods

To measure TLF-1 binding by flow cytometry, cells were grown to mid-log phase, collected, washed and resuspended (1×10⁷/ml) in DMEM supplemented with 10% fetal bovine serum. Alexa-488 TLF-1, labeled according to manufacture instructions (Invitrogen), was incubated with an excess of Hb for 10 minutes on ice then added to cells in ice-cold complete DMEM and further incubated at 3° C. for three hours. Cells were washed two times with ice-cold phosphate buffered saline buffer (PBS) (10 mM NaPi, 137 mM NaCl, pH 7.4), kept on ice analyzed by flow cytometry. All binding experiments were done in triplicate with 50,000 cells measured per experiment/data point.

### Results

Investigations into the mechanisms that allow mammalian cells to tolerate TLF have never been carried out. HDL endocytosis has been studied in multiple systems and the precise purpose and efficiency of this process is under debate, but the transport of cholesterol is thought to be one of the primary goals (Rohrl and Stangl, “HDL endocytosis and resecretion” Biochim Biophys Acta, 2013. 1831(11): p. 1626-1633). The question of TLF endocytic efficiency also has not been thoroughly examined aside from a single observation that TLF was internalized in macrophage infected with Leishmania (Samanovic, et al., “Trypanosome lytic factor, an antimicrobial high-density lipoprotein, ameliorates Leishmania infection,” PLoS Pathog, 2009. 5(1): p. e1000276). In this case, no macrophage cell death was reported, thereby indicating that TLF, when at approximate physiological levels in culture, was somehow unable to elicit its toxic effects.

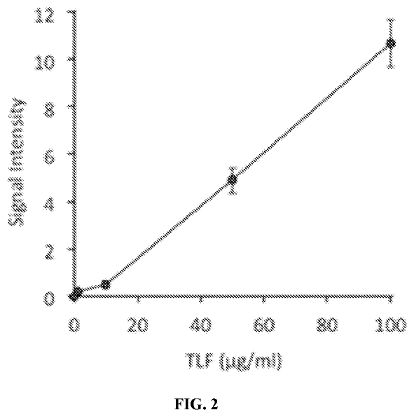

Trypanosoma brucei brucei binds TLF with high specificity and affinity and can be observed easily at 3 C by a previously developed method (DeJesus, et al., “A Single Amino Acid Substitution in the Group 1 Trypanosoma brucei gambiense Haptoglobin-Hemoglobin Receptor Abolishes TLF-1 Binding,” PLoS Pathog., 9 (2013)). To identify if a receptor existed for TLF in mammalian cells, this same low temperature binding assay was carried out. While no high affinity binding was observed, high concentrations of AF488 TLF-1 were detectable but not saturable by flow cytometry (FIG. 2). See also, Dejesus, et al., “Evasion of African trypanosomes to human innate immunity,” Dissertation in fulfillment of Doctor of Philosophy, University of Georgia, submitted 2014.

### Example 2: TLF is Internalized and Localizes to Lysosomes in Mammalian Cells

### Materials and Methods

### TLF-1 Binding and Uptake Studies

To measure TLF-1 uptake by flow cytometry, cells were grown to mid-log phase, collected, washed, and resuspended (1×10⁷/ml) in DMEM supplemented with 10% fetal bovine serum. Alexa-488 TLF-1 done both with and without hemoglobin, were added to the cells followed by incubation at 37° C. for three hours. Uptake was stopped by placing the tubes on ice followed by two washes with ice-cold PBS. The amount of TLF-1 uptake was determined using both Cyan cytometer and Amnis ImageStream cytometer with analysis from FlowJo software. For uptake studies, 20,000 cells were imaged through Amnis Imagestream per experiment, each experiment done in triplicate. Uptake was also measured by fluorescence microscopy. Following incubation, cells were washed two times with ice cold PBS. Following the washes, cells were spread onto glass slides, methanol-fixed for 5 min, at −20° C., and analyzed by fluorescence microscopy. Images were captured by Zeiss Image Capture Inverted Microscope and Axiovision v4.6 software. The images were subjected to the same exposure and were contrasted to the same extent.

### Competition Binding Studies

Specificity of TLF-1 binding to HEK293 cells was analyzed using competition-binding studies with the unlabeled Non-lytic HDLs and Hp 1-1. Cells were collected, washed and resuspended (1×10⁷/ml) in ice-cold DMEM supplemented with 10% fetal bovine serum then transferred to 3° C. for at least 10 minutes. Alexa-488 conjugated TLF-1 (20 nM constant) was complexed with hemoglobin (50 nM) at 4° C. for 10 minutes. Increasing concentrations of unlabeled competitor were incubated with Hb (50 nM) for 10 minutes at 4° C. Competing ligands were then mixed with the Alexa-488 conjugated TLF-1/Hb, added to cells at 3° C. and allowed to incubate for three hours. Cells were then transferred to ice, washed with ice-cold 1×PBS and analyzed by Cyan cytometer and FlowJo software. All competition studies were done in triplicate.

### Results

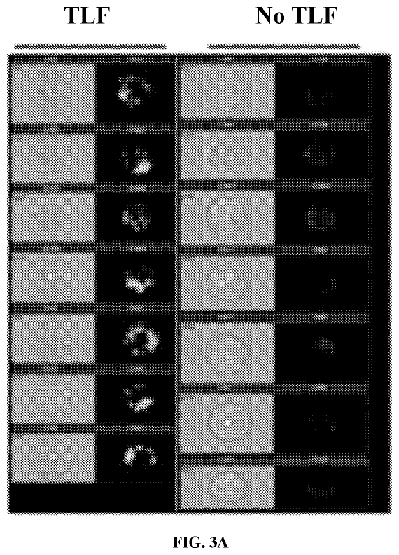

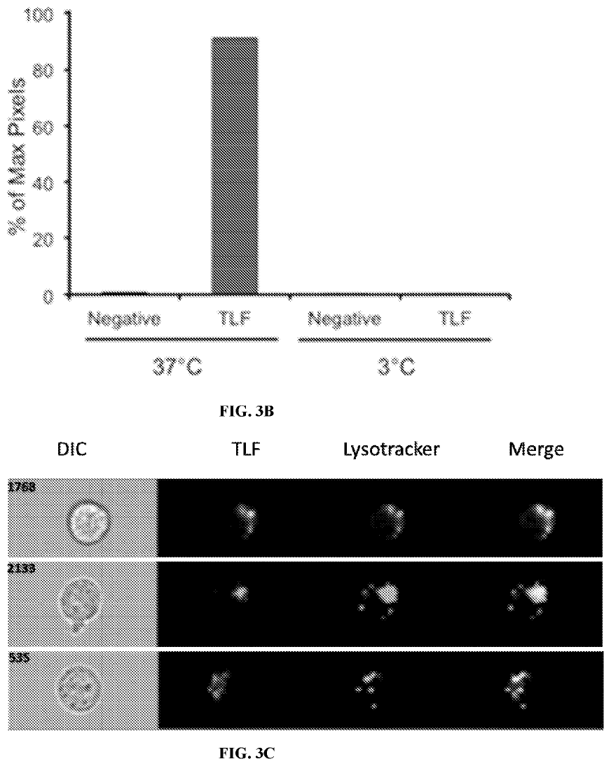

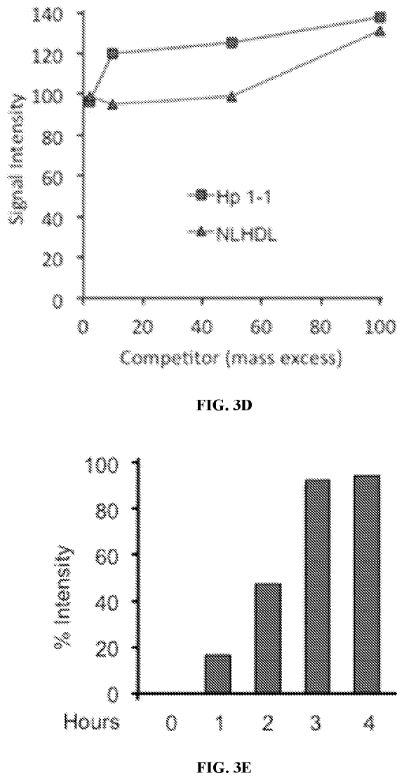

Next, experiments were designed to investigate TLF uptake in HEK293 mammalian cells. First, cells were incubated with AF488 TLF at 37° C. and imaged via Amnis ImageStream. Amnis Internalization plotting analysis was then used to identify particle location. AF488 TLF was observed being endocytosed into vesicles (FIGS. 3A and 31B) with the maximum pixel intensity indicating TLF to be inside the cell, not cell-surface associated.

After determining that TLF was indeed taken up by HEK293 cells, experiments were designed to determine TLF cellular localization. Colocalization with AF488 TLF and lysotracker indicate that TLF does localize to low pH compartments resembling the lysosome (FIG. 3C). This was further confirmed with fluorescence microscopy. To test whether TLF uptake was due to a specific Haptoglobin (Hp) receptor (as it is in Trypanosoma brucei brucei), a competition-binding assay with unlabeled ligand was performed. As indicated in FIG. 3D, no competition with increasing amounts of the unlabeled Hp (both by molar and mass equivalents) was observed. These findings agree with previously published literature for HDL binding in mammalian cells, including HEK293 cells, that, unlike human infective trypanosomes, there is no TLF-specific receptor in mammalian systems (Xiao, et al., Circ Res., 103:159-66 (2008)).

Previous studies using HEK293 cells transfected with Scavenging Receptor-Class B type I (SR-BI, a haptoglobin-hemoglobin receptor) have measured SR-BI mediated HDL uptake to reach saturation within three hours (Pagler, et al., J Biol Chem., 281:11193-204 (2006)). To test if wild type HEK293 cellular endocytic machinery could reach equilibrium, a TLF uptake time course was carried out. By three hours, the signal for AF488-TLF plateaued, indicating equilibrium (FIG. 3E).

Hemoglobin has been shown to be an important co-factor for TLF binding and uptake in African trypanosomes (Widener, et al., PLoS Pathog. 3:e129 (2007)). As discussed and illustrated herein, a receptor capable of binding the Hpr present in TLF has not been identified in mammalian systems. Considering this, experiments were designed to examine differences in the rates of uptake when hemoglobin was added. The intensity of the signal of AF488 TLF, analyzed using flow cytometry, did not indicate any substantial difference in the rate in uptake. See also, Dejesus, et al., “Evasion of African trypanosomes to human innate immunity,” Dissertation in fulfillment of Doctor of Philosophy, University of Georgia, submitted 2014.

### Example 3: Mammalian Cells are Sensitive to TLF and Recombinant ApoL1

### Materials and Methods

### Cell Survival Assay

### HEK293

HEK293 cells were harvested from mid-log phase cultures, washed and re-suspended at a final concentration of 1×106/ml in complete DMEM media. Susceptibility to hemoglobin (Hb) bound TLF was determined over a range of TLF concentrations following incubation at 37° C. for 72 hours. The number of surviving cells was determined by hemocytometer count with phase contrast microscopy. Additionally, cell viability was quantified by 4flow cytometry (Cyan) using the LIVE/DEAD Cell viability kit (Invitrogen). All survival assays were done in triplicate.

### Cell Culture and Maintenance

Cells were cultivated in the indicated growth media. Growth media: RPMI-1640 with 10% FBS for RPMI8226 (CCL-155), K-562 (CCL-243); McCoy's 5A with 10% FBS, 1% L-glutamine, 1% antibiotic/antimycotic for HT144; DMEM with 10% FBS, 1% L-glutamine, 1% antibiotic/antimycotic for Panc1 and A375. Cells were maintained at 37° C. in a humidified atmosphere with 5% CO2.

### RPMI 8226 ATCC CCL-155 Cells

Cells were cultivated in RPMI-1640 with 10% FBS, 1% antibiotic/antimycotic. Cells were inoculated in white 384 well plates in a total of 40 μl with a final cell density of 1e5/mL and allowed to grow with indicated additives for four days. At completion of the experiment, cells were processed using the Cell Titer Cell Survivability Assay.

### PANC-1 ATCC CRL-1469

Cells were cultivated in DMEM with 10% FBS, 1% L-glutamine, 1% antibiotic/antimycotic. Cells were inoculated in white 384 well plates in a total of 80 μl with a final cell density of 5e4/mL and allowed to grow and attach overnight. Next day, media was removed and replaced with 50 μl complete media with indicated additives and allowed to grow for four days. At completion of the experiment, cells were processed using the Cell Titer Cell Survivability Assay.

### A375/ATCC CRL-1619

Cells were cultivated in DMEM with 10% FBS, 1% L-glutamine, 1% antibiotic/antimycotic. Cells were inoculated in white 384 well plates in a total of 80 μl with a final cell density of 5e4/mL and allowed to grow and attach overnight. Next day, media was removed and replaced with complete media with indicated additives and allowed to grow for four days. At completion of the experiment, cells were processed using the Cell Titer Cell Survivability Assay.

### HT144 ATCC HTB-63

Cells were cultivated in McCoy's with 10% FBS, 1% L-glutamine, 1% antibiotic/antimycotic. Cells were inoculated in white 384 well plates in a total of 80 μl with a final cell density of 5×10∝cell/mL and allowed to grow and attach overnight. Next day, media was removed and replaced with complete media with indicated additives and allowed to grow for three days. At completion of the experiment, cells were processed using the Cell Titer Cell Survivability Assay.

### Cell Titer Cell Survivability Assay

Equal volume CellTiter-Glo 2.0 (Promega Cat. G9241) was added to each well via robotic injector. Plates were allowed to shake for 1 minute and luminescence is measured after 10 minutes via SpectraMax iD3 plate reader (Molecular Diagnostics). Data were imported into GraphPad, and standard error and t-test performed to measure β-values indicated. Float Viability

### Assay

Human HDLs were purified by differential floatation on a sodium bromide gradient (1.26 float/float) as previously described (1). Float was diluted with PBS to the indicated total protein content and mixed 1:1 with cells in a standard Cell Survival Assay.

### Recombinant ApoL1 Cell Viability Assays

Recombinant ApoL1 (Sino Biological 13910-H08B) was resuspended in complete media at 100 μg/mL, and added to each well at the indicated final concentrations. Five replicates for each concentration were performed.

### ApoL1 Depletion Assays

Human HDLs were purified by differential floatation on a sodium bromide gradient (1.26 float/float) as previously described (Shiflett, et al., J Biol Chem 280:32578-32585 (2005)). One mL Float was incubated with indicated antibodies (anti-ApoL1 13.11, anti-Hpr 14.11 (antibody sequences provided below)) at 150 μg/mL, 75 μg/mL, 37 μg/mL, 0 μg/mL overnight at 4 C on rotator. 100 μl Protein-G MagBeads (Genscript L00274) was added to each sample and incubated on a rotator at RT for 1 hour. Beads were removed via magnet and depleted float used in Cell Survival Assay at six replicates per condition.

### Results

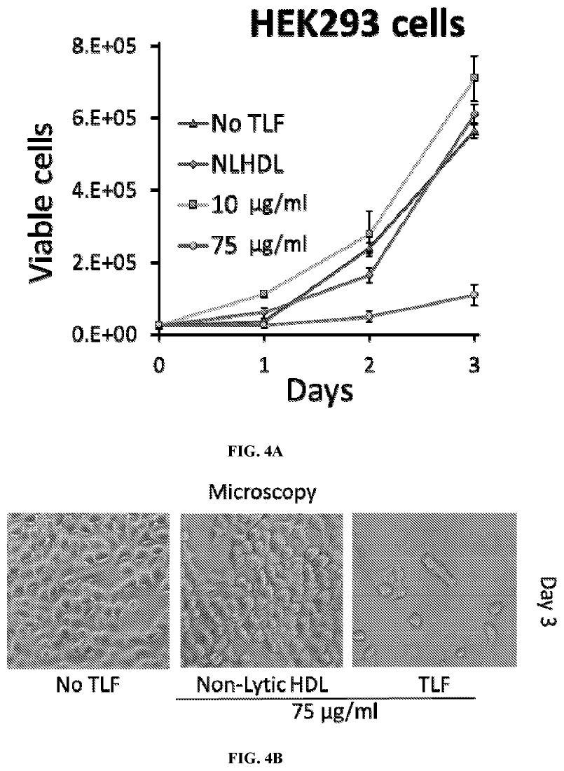

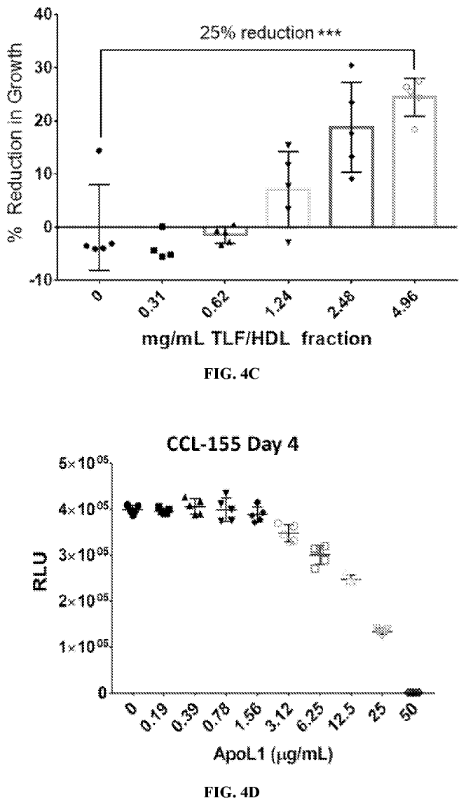

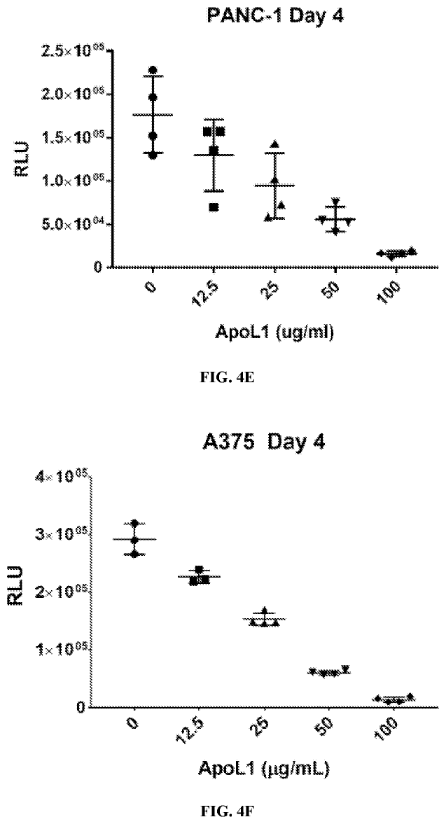

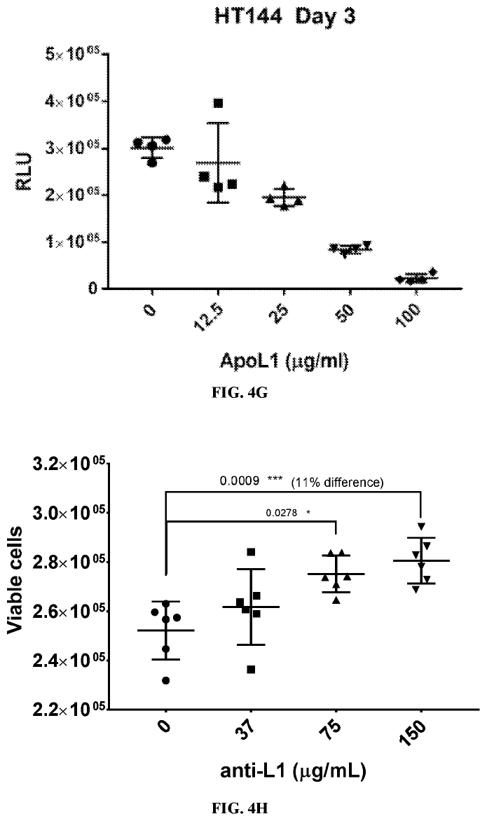

To test whether mammalian cells would be resistant to TLF in culture, a 72-hour survival assay was carried out. Concentrations of TLF up to 20 μg/ml showed no inhibition in cell growth (FIG. 4A). However, incubation with 75 μg/ml TLF for 72 hours caused attenuation of growth. Physiological levels of TLF are approximately 10 μg/ml (Samanovic, et al., PLoS Pathog., 5:e1000276 (2009)). The difference in cell density between the 24-hour mark and the 72-hour was also recorded by light microscopy (FIG. 4B). See also, Dejesus, et al., “Evasion of African trypanosomes to human innate immunity,” Dissertation in fulfillment of Doctor of Philosophy, University of Georgia, submitted 2014.

Next, RPMI 8226 (CCL-155) multiple myeloma (MM) cells were incubated with high concentration TLF fraction. Increases in high concentration TLF/HDL fraction result in reduced viability of cells as measured by CellTiter-Glo (25% reduction at 4.96 mg/mL) (FIG. 4C).

RPMI 8226 (CCL-155, Multiple Myeloma), PANC-1 (CRL-1469, human pancreatic cancer), A375 (CRL-1619, human malignant melanoma), and HT144 (HTB-63, human malignant melanoma) cells were incubated with recombinant ApoL1 between 3-4 days (FIG. 4D-4G). Cell viability was assayed on day three for HT-144 and day four for CCL-155, PANC-1 and A375, using CellTiter-Glo. The determination of LD50 values was achieved through the utilization of the Quest Graph EC50 Calculator, employing a four-parameter model. The resultant LD50 values for different cell lines were as follows: CCL-155 at 20.2 μg/mL, PANC-1 at 43 μg/mL, A375 at 27.5 μg/mL, and HT144 at 32.8 μg/mL. Data shows a dose dependent effect of recombinant ApoL1 on cell growth. For comparison, circulating levels of ApoL1 range from ˜400 ng-15 μg, with a median of 3 μg (Bruggeman, et al., J Am Soc Nephrol., 25:634-44 (2014)).

Depletion of TLF with ApoL1 mAb increases survival of RPMI 8226 (CCL-155) multiple myeloma cell line (FIG. 4H). This data shows that by increasing the amounts of anti-ApoL1 mAb added during immunoprecipitation the cell growth effect of ApoL1 containing HDL particles can be ablated. Similar results were obtained when Anti-Hpr antibody was used (FIG. 4I).

### Example 4: Anti-Hpr Antibody SFII 134.3 Sequences and Binding Assays

### Materials and Methods

### Antibody Sequencing

Sequencing of SFII 134.3, Mouse anti-Hpr IgG2a, was performed by whole transcriptome shotgun sequencing (RNA-Seq). Total RNA was extracted from hybridoma cells and a barcoded cDNA library generated through RT-PCR using a random hexamer. Next Generation Sequencing was performed on an Illumina HiSeq sequencer. Contigs were assembled and data mined for antibody sequences identifying all viable antibody sequences (i.e. those not containing stop codons). Variable heavy and variable light domains were identified separately.

Genes have been separated into heavy chains and light chains. For each chain the variable domain is reported along with the signal peptide and constant domain region.

The complementarity determining regions (CDRs) have been identified. Sequences according to the Kabat, IMGT, and Chothia formulas for CDRs are provided.

### Western Blots

Materials: TLF—Column Purified TLF (2 ug), rApoL1—ApoL1 Protein, human recombinant (Sino biological 13910-H08B) (lug), L—Chameleon Duo Ladder (Licor) −8 uL, L2—Benchmark Protein Ladder (Thermo 10747012) −2 uL
Assay: SDS-Page 4%-15% Mini-PROTEAN TGX (200V, 30 min), transferred to nitrocellulose (75V, 45 min), Block 1 hour in Intercept blocking buffer (PBS), 1:10,000 dilution of antibody in 0.5× Intercept blocking buffer (PBS)+0.2% Tween−Overnight, Wash 4×PBS-T, 1:10,000 dilution of secondary antibody in 0.5× Intercept blocking buffer (PBS)+0.2% Tween−1 hour (Licor goat anti-mouse 800, 926-32210), Wash 4× PBS-T, Rinse 1×PBS, Image on Odyssey DLx Infrared Imager in the 800 channel.

### Dot Blots

Materials: rApoL1—ApoL1 Protein, human recombinant (Sino biological 13910-H08B) (lug)
Assay: 500 ng ApoL1 in 10 ul 50% FBS, Half dilutions across dots in 50% FBS, Block 1 hour in Intercept blocking buffer (PBS), Wash 4×PBS-T, 1:10,000 dilution of antibody in 0.5× Intercept blocking buffer (PBS)+0.2%, Tween—Overnight, Wash 4×PBS-T, 1:10,000 dilution of secondary antibody in 0.5× Intercept blocking buffer (PBS)+0.2% Tween−1 hour (Licor goat anti-mouse 800, 926-32210), Wash 4×PBS-T, Rinse 1×PBS, Image on Odyssey DLx Infrared Imager in the 800 channel.

### Results

Signal Peptide Amino Acid and Nucleic Acid
Sequences
(SEQ ID NO: 1)
MAWVWTLLELMAAAQSAQA
(SEQ ID NO: 2)
ATGGCTTGGGTGTGGACCTTGCTATTCCTGATGGCAGCTGCCCAAAGTG
CCCAAGCA
Heavy Chain Variable Domain (VH) and CDR Amino
Acid and Nucleic Acid Sequences
(SEQ ID NO: 3)
QIQLVQSGPELKKPGETVKISCKASGYIFTNYGMNWVRQAPGKGLKWMG
WINSYTGEATYTDDLKGRFAFSLESSASTAYLQINNLKNEDTATYFCAR
EGYGDYGYSFDYWGQGTTLTVSS

### Complementary Determining Region Sequences

| Scheme | CDR-H1 | CDR-H2 | CDR-H3 |
|---|---|---|---|
| Kabat | NYGMN (SEQ ID | WINSYTGEATYTDDLKG | EGYGDYGYSFDY |
| | NO: 4) | (SEQ ID NO: 5) | (SEQ ID NO: 6) |
| IMGT | GYIFTNYG (SEQ | INSYTGEA (SEQ ID | AREGYGDYGYSFDY |
| | ID NO: 7) | NO: 8) | (SEQ ID NO: 9) |
| Chothia | GYIFTNY (SEQ | NSYTGE (SEQ ID | EGYGDYGYSFDY |
| | ID NO: 10) | NO: 11) | (SEQ ID NO: 6) |

CAGATCCAGTTGGTGCAGTCTGGACCTGAGCTGAAGAAGCCTGGAGAGACA
GTCAAGATCTCCTGCAAGGCTTCTGGATATATTTTCACAAACTATGGAATG
AACTGGGTGAGACAGGCTCCGGGAAAGGGTTTAAAGTGGATGGGCTGGATA
AACTCCTACACTGGAGAGGCAACATATACTGACGACCTCAAGGGACGGTTT
GCCTTCTCTTTGGAATCCTCTGCCAGCACTGCCTATTTGCAGATCAACAAC
CTCAAAAATGAGGACACGGCTACTTATTTCTGTGCAAGAGAGGGATATGGT
GACTACGGGTACTCCTTTGACTACTGGGGCCAAGGCACCACTCTCACAGTC
TCCTCA (SEQ ID NO: 78)
Heavy Chain Constant Amino Acid and Nucleic Acid Sequences
AKTTAPSVYPLAPVCGDTTGSSVTLGCLVKGYFPEPVTLTWNSGSLSSGVH
TFPAVLQSDLYTLSSSVTVISSTWPSQSITCNVAHPASSTKVDKKIEPRGP
TIKPCPPCKCPAPNLLGGPSVFIFPPKIKDVLMISLSPIVTCVVVDVSEDD
PDVQISWFVNNVEVHTAQTQTHREDYNSTLRVVSALPIQHQDWMSGKEFKC
KVNNKDLPAPIERTISKPKGSVRAPQVYVLPPPEEEMTKKQVTLTCMVTDE
MPEDIYVEWTNNGKTELNYKNTEPVLDSDGSYFMYSKLRVEKKNWVERNSY
SCSVVHEGLHNHHTTKSESRTPGK (SEQ ID NO: 12)
GCCAAAACAACAGCCCCATCGGTCTATCCACTGGCCCCTGTGTGTGGAGAT
ACAACTGGCTCCTCGGTGACTCTAGGATGCCTGGTCAAGGGTTATTTCCCT
GAGCCAGTGACCTTGACCTGGAACTCTGGATCCCTGTCCAGTGGTGTGCAC
ACCTTCCCAGCTGTCCTGCAGTCTGACCTCTACACCCTCAGCAGCTCAGTG
ACTGTAACCTCGAGCACCTGGCCCAGCCAGTCCATCACCTGCAATGTGGCC
CACCCGGCAAGCAGCACCAAGGTGGACAAGAAAATTGAGCCCAGAGGGCCC
ACAATCAAGCCCTGTCCTCCATGCAAATGCCCAGCACCTAACCTCTTGGGT
GGACCATCCGTCTTCATCTTCCCTCCAAAGATCAAGGATGTACTCATGATC
TCCCTGAGCCCCATAGTCACATGTGTGGTGGTGGATGTGAGCGAGGATGAC
CCAGATGTCCAGATCAGCTGGTTTGTGAACAACGTGGAAGTACACACAGCT
CAGACACAAACCCATAGAGAGGATTACAACAGTACTCTCCGGGTGGTCAGT
GCCCTCCCCATCCAGCACCAGGACTGGATGAGTGGCAAGGAGTTCAAATGC
AAGGTCAACAACAAAGACCTCCCAGCGCCCATCGAGAGAACCATCTCAAAA
CCCAAAGGGTCAGTAAGAGCTCCACAGGTATATGTCTTGCCTCCACCAGAA
GAAGAGATGACTAAGAAACAGGTCACTCTGACCTGCATGGTCACAGACTTC
ATGCCTGAAGACATTTACGTGGAGTGGACCAACAACGGGAAAACAGAGCTA
AACTACAAGAACACTGAACCAGTCCTGGACTCTGATGGTTCTTACTTCATG
TACAGCAAGCTGAGAGTGGAAAAGAAGAACTGGGTGGAAAGAAATAGCTAC
TCCTGTTCAGTGGTCCACGAGGGTCTGCACAATCACCACACGACTAAGAGC
TTCTCCCGGACTCCGGGTAAA (SEQ ID NO: 13)
Light Chain Variable Domain (VL) and CDR Amino Acid and
Nucleic Acid Sequences
DIQMTOSPASLSASVGETVTITCRATKNIYTYLAWYQQKOGKSPQELVYNA
KTLAEGVPSRFSGSGSGTQFSLKINSLQPEDFGNYYCQHHYGTPRTFGGGT
KLEIK (SEQ ID NO: 14)

### Complementary Determining Region Sequences

| Scheme | CDR-L1 | CDR-L2 | CDR-L3 |
|---|---|---|---|
| Kabat | RATKNIYTYLA (SEQ | NAKTLAE (SEQ ID | QHHYGTPRT |
| | ID NO: 16) | NO: 17) | (SEQ ID NO: 18) |
| IMGT | KNIYTY (SEQ ID | NAK | QHHYGTPRT |
| | NO: 19) | | (SEQ ID NO: 18) |
| Chothia | RATKNIYTYLA (SEQ | NAKTLAE (SEQ ID | QHHYGTPRT |
| | ID NO: 16) | NO: 17) | (SEQ ID NO: 18) |

(SEQ ID NO: 15)
GACATCCAGATGACTCAGTCTCCAGCCTCCCTATCTGCATCTGTGGGGGAA
ACTGTCACCATCACATGTCGAGCAACTAAGAATATTTACACTTATTTAGCA
TGGTATCAGCAGAAACAGGGAAAATCTCCTCAGTTCCTGGTCTATAATGCA
AAAACCTTAGCAGAAGGTGTGCCATCAAGGTTCAGTGGCAGTGGATCAGGC
ACACAGTTTTCTCTGAAGATCAATAGCCTGCAGCCTGAAGATTTTGGGAAT
TATTACTGTCAACATCATTATGGAACTCCTCGGACGTTCGGTGGAGGCACC
AAGCTGGAAATCAAA
Light Chain Constant Amino Acid and Nucleic Acid
Sequences
(SEQ ID NO: 20)
RADAAPTVSIFPPSSEQLTSGGASVVCFLNNFYPKDINVKWKIDGSERQNG
VLNSWTDQDSKDSTYSMSSTLTLTKDEYERHNSYTCEATHKTSTSPIVKSF
NRNEC
(SEQ ID NO: 21)
CGGGCTGATGCTGCACCAACTGTATCCATCTTCCCACCATCCAGTGAGCAG
TTAACATCTGGAGGTGCCTCAGTCGTGTGCTTCTTGAACAACTTCTACCCC
AAAGACATCAATGTCAAGTGGAAGATTGATGGCAGTGAACGACAAAATGGC
GTCCTGAACAGTTGGACTGATCAGGACAGCAAAGACAGCACCTACAGCATG
AGCAGCACCCTCACGTTGACCAAGGACGAGTATGAACGACATAACAGCTAT
ACCTGTGAGGCCACTCACAAGACATCAACTTCACCCATTGTCAAGAGCTTC
AACAGGAATGAGTGT

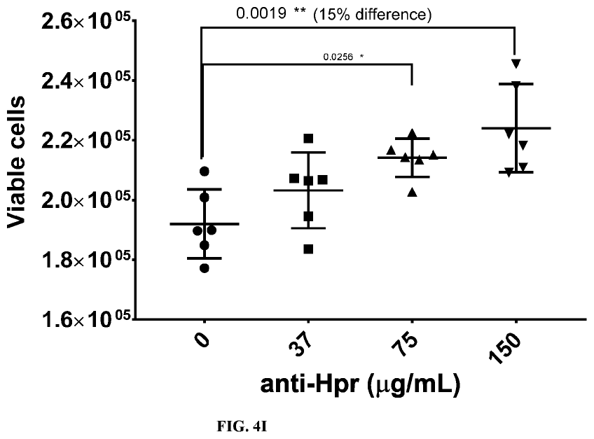

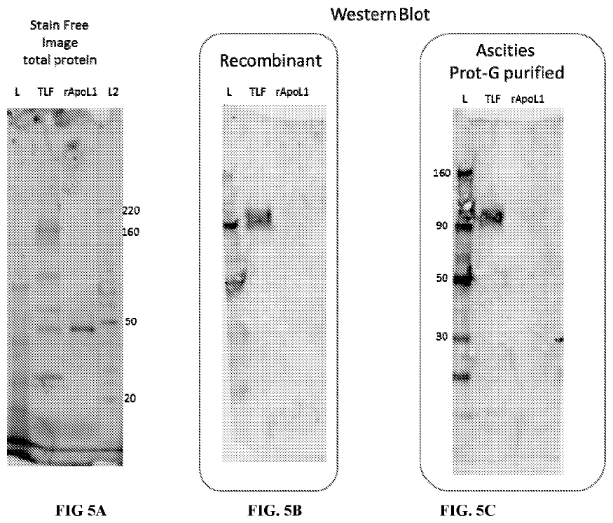

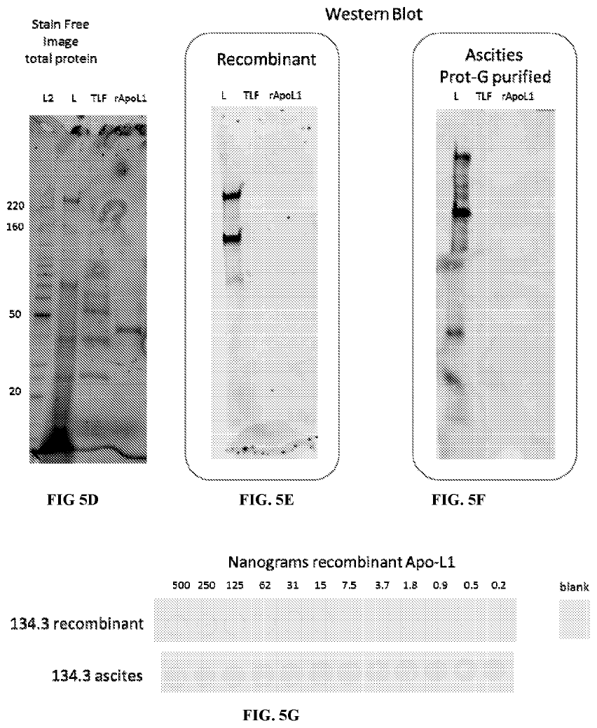

A Western blot assay comparing recombinant antibody and ascites Pro-G purified antibody, shows that both recombinant and purified antibodies bind in the TLF column, not the rApoL1, under non-reducing, but not reducing conditions, and the size correlates with Hpr from previous assays. See FIGS. 5A-5F. Neither antibody bound recombinant ApoL1 in a Dot Blot (native) assay. See FIG. 5G.

### Example 5: Anti-Hpr Antibody SFII 14.11 Sequences

### Materials and Methods

### Antibody Sequencing

Hybridoma SFII 14.11 antibody sequencing of CDR's (variable region only)
Reverse transcription (RACE) with VL and VH PCR primers
Subclone PCR products into plasmids and express into single colonies
DNA gel verification; sequencing validated with 5 colonies

The complementarity determining regions (CDRs) have been identified. Sequences according to the Kabat, IMGT, and Chothia formulas for CDRs are provided.

### Results

Heavy Chain Variable Domain (VH) and CDR Amino Acid
and Nucleic Acid Sequences
(SEQ ID NO: 56)
QIQLVQSGPELKKPGETVKISCKASGFTFTDYSIHWVKQAPGKGLKWMGWK
HTESGESTYADDFKGRFVFSLETSASTAYLQINNLKNEDTSTYFCARGANY
GSLLDYWGQGTTLTVSS

### Complementary Determining Region Sequences

| Scheme | CDR-H1 | CDR-H2 | CDR-H3 |
|---|---|---|---|
| Kabat | DYSIH (SEQ ID | WKHTESGESTYADDFKG | GANYGSLLDY (SEQ |
| | NO: 57) | (SEQ ID NO: 58) | ID NO: 59) |
| IMGT | GFTFTDYS | KHTESGES (SEQ ID | ARGANYGSLLDY |
| | (SEQ ID | NO: 61) | (SEQ ID NO: 62) |
| | NO: 60) | | |
| Chothia | GFTFTDY | HTESGE (SEQ ID | GANYGSLLDY (SEQ |
| | (SEQ ID | NO: 64) | ID NO: 59) |
| | NO: 63) | | |

(SEQ ID NO: 79)
CAGATCCAGTTGGTGCAGTCTGGACCTGAACTGAAGAAGCCTGGAGAGACA
GTCAAGATCTCCTGCAAGGCTTCTGGTTTTACCTTCACAGACTATTCAATC
CACTGGGTGAAGCAGGCTCCAGGAAAGGGTTTAAAGTGGATGGGCTGGAAA
CACACTGAAAGTGGTGAGTCAACATATGCAGATGACTTCAAGGGACGGTTT
GTCTTCTCTTTGGAAACCTCTGCCAGCACTGCCTATTTGCAGATCAACAAC
CTCAAAAATGAGGACACGTCTACATATTTCTGTGCTAGGGGGGCGAACTAT
GGTTCCCTACTTGACTACTGGGGCCAAGGCACCACTCTCACAGTCTCCTCA
Light Chain Variable Domain (VL) and CDR Amino Acid
and Nucleic Acid Sequences
(SEQ ID NO: 65)
DIVLTQSPASLAVSLGQRATISCRASKSVSTSGYSYMHWYQQKPGQSPKLL
IYLASNLESGVPARFSGSGSGTDFTLNIHPVEEEDAATYYCQHNRELPLTF
GAGTKLELKR

Complementary Determining Region Sequences

| Scheme | CDR-L1 | CDR-L2 | CDR-L3 |
|---|---|---|---|
| Kabat | RASKSVSTSGYSYMH | LASNLES (SEQ | QHNRELPLT (SEQ ID |
| | (SEQ ID NO: 66) | ID NO: 67) | NO: 68) |
| IMGT | KSVSTSGYSY (SEQ ID | LAS | QHNRELPLT (SEQ ID |
| | NO: 69) | | NO: 68) |
| Chothia | RASKSVSTSGYSYMH | LASNLES (SEQ | QHNRELPLT (SEQ ID |
| | (SEQ ID NO: 66) | ID NO: 67) | NO: 68) |

(SEQ ID NO: 70)
GACATTGTGCTGACACAGTCTCCTGCTTCCTTAGCTGTATCTCTGGGGCAG
AGGGCCACCATCTCATGCAGGGCCAGCAAAAGTGTCAGTACATCTGGCTAT
AGTTATATGCACTGGTACCAACAGAAACCAGGACAGTCACCCAAACTCCTC
ATCTATCTTGCATCCAACCTAGAATCTGGGGTCCCTGCCAGGTTCAGTGGC
AGTGGGTCTGGGACAGACTTCACCCTCAACATCCATCCTGTGGAGGAGGAG
GATGCTGCAACCTATTACTGTCAGCACAATAGGGAGCTTCCGCTCACGTTC
GGTGCTGGGACCAAGCTGGAGCTGAAACGG

### Example 6: Anti-ApoL1 Antibody Sequences and Binding Assays

### Materials and Methods

### Antibody Sequencing

Sequencing of SFIII 13.11, Mouse Anti-ApoL1 IgG1, was performed by whole transcriptome shotgun sequencing (RNA-Seq). Total RNA was extracted from hybridoma cells and a barcoded cDNA library generated through RT-PCR using a random hexamer. Next Generation Sequencing was performed on an Illumina HiSeq sequencer. Contigs were assembled and data mined for antibody sequences identifying all viable antibody sequences (i.e. those not containing stop codons). Variable heavy and variable light domains were identified separately.

Genes have been separated into heavy chains and light chains. For each chain the variable domain is reported along with the signal peptide and constant domain region.

The complementarity determining regions (CDRs) have been identified. Sequences according to the Kabat, IMGT, and Chothia formulas for CDRs are provided.

### Western Blots

Materials: TLF—Column Purified TLF (2 ug), rApoL1—ApoL1 Protein, human recombinant (Sino biological 13910-H08B) (lug), L—Chameleon Duo Ladder (Licor) −8 uL, L2—Benchmark Protein Ladder (Thermo 10747012) −2 uL
Assay: SDS-Page 4%-15% Mini-PROTEAN TGX (200V, 30 min), transferred to nitrocellulose (75V, 45 min), Block 1 hour in Intercept blocking buffer (PBS), 1:10,000 dilution of antibody in 0.5× Intercept blocking buffer (PBS)+0.2% Tween−Overnight, Wash 4×PBS-T, 1:10,000 dilution of secondary antibody in 0.5× Intercept blocking buffer (PBS)+0.2% Tween−1 hour (Licor goat anti-mouse 800, 926-32210), Wash 4× PBS-T, Rinse 1×PBS, Image on Odyssey DLx Infrared Imager in the 800 channel.

### Dot Blots

Materials: rApoL1—ApoL1 Protein, human recombinant (Sino biological 13910-H08B) (lug)
Assay: 500 ng ApoL1 in 10 ul 50% FBS, Half dilutions across dots in 50% FBS, Block 1 hour in Intercept blocking buffer (PBS), Wash 4×PBS-T, 1:10,000 dilution of antibody in 0.5× Intercept blocking buffer (PBS)+0.2%, Tween−Overnight, Wash 4×PBS-T, 1:10,000 dilution of secondary antibody in 0.5× Intercept blocking buffer (PBS)+0.2% Tween−1 hour (Licor goat anti-mouse 800, 926-32210), Wash 4×PBS-T, Rinse 1×PBS, Image on Odyssey DLx Infrared Imager in the 800 channel.

### Results

Signal Peptide Amino Acid and Nucleic Acid
Sequences
(SEQ ID NO: 22)
MNFGLSLIFLVLILKGVQC
(SEQ ID NO: 23)
ATGAACTTCGGGCTCAGCTTGATTTTCCTTGTCCTCATTTTAAAAGGTGT
CCAGTGT
Heavy Chain Variable Domain (VH) and CDR Amino
Acid and Nucleic Acid Sequences
(SEQ ID NO: 24)
EVQLVESGGGLVKPGGSLKLSCAASGFTFSTYAMSWVRQSPEKRLEWVAE
ISNGGLYTYYPDTVTGRFTISRDNVKNILYLEMSSLRSEDTAIYYCIREN
RNWYFDLWGAGTTVTVSS

### Complementary Determining Region Sequences

| Scheme | CDR-H1 | CDR-H2 | CDR-H3 |
|---|---|---|---|
| Kabat | TYAMS (SEQ ID | EISNGGLYTYYPDTVTG | ENRNWYFDL (SEQ |
| | NO: 25) | (SEQ ID NO: 26) | ID NO: 27) |
| IMGT | GFTFSTYA | ISNGGLYT (SEQ ID | IRENRNWYFDL |
| | (SEQ ID NO: 28) | NO: 29) | (SEQ ID NO: 30) |
| Chothia | GFTFSTY (SEQ | SNGGLY (SEQ ID | ENRNWYFDL (SEQ |
| | ID NO: 31) | NO: 32) | ID NO: 27) |

(SEQ ID NO: 33)
GAAGTGCAGCTGGTGGAGTCTGGGGGAGGCTTAGTGAAGCCTGGAGGGTCC
CTGAAACTCTCCTGTGCAGCCTCTGGATTCACCTTCAGTACCTATGCCATG
TCTTGGGTTCGCCAGTCTCCAGAGAAGAGGCTGGAGTGGGTCGCAGAAATT
AGTAATGGTGGTCTTTACACCTACTATCCAGACACTGTGACGGGCCGATTC
ACCATCTCCAGAGACAATGTCAAGAATATCTTATACCTGGAAATGAGCAGT
CTGAGGTCTGAGGACACGGCCATATATTACTGTATAAGAGAAAATAGGAAC
TGGTACTTCGATCTCTGGGGCGCAGGGACCACGGTCACCGTCTCCTCA
Heavy Chain Constant Amino Acid and Nucleic Acid
Sequences
(SEQ ID NO: 34)
AKTTPPSVYPLAPGSAAQTNSMVTLGCLVKGYFPEPVTVTWNSGSLSSGVH
TFPAVLQSDLYTLSSSVTVPSSTWPSETVTCNVAHPASSTKVDKKIVPRDC
GCKPCICTVPEVSSVFIFPPKPKDVLTITLTPKVTCVVVDISKDDPEVQFS
WFVDDVEVHTAQTQPREEQENSTERSVSELPIMHQDWLNGKEFKCRVNSAA
FPAPIEKTISKTKGRPKAPQVYTIPPPKEQMAKDKVSLTCMITDFFPEDIT
VEWQWNGQPAENYKNTQPIMDTDGSYFVYSKLNVQKSNWEAGNTFTCSVLH
EGLHNHHTEKSLSHSPGK
(SEQ ID NO: 35)
GCCAAAACGACACCCCCATCTGTCTATCCACTGGCCCCTGGATCTGCTGCC
CAAACTAACTCCATGGTGACCCTGGGATGCCTGGTCAAGGGCTATTTCCCT
GAGCCAGTGACAGTGACCTGGAACTCTGGATCCCTGTCCAGCGGTGTGCAC
ACCTTCCCAGCTGTCCTGCAGTCTGACCTCTACACTCTGAGCAGCTCAGTG
ACTGTCCCCTCCAGCACCTGGCCCAGCGAGACCGTCACCTGCAACGTTGCC
CACCCGGCCAGCAGCACCAAGGTGGACAAGAAAATTGTGCCCAGGGATTGT
GGTTGTAAGCCTTGCATATGTACAGTCCCAGAAGTATCATCTGTCTTCATC
TTCCCCCCAAAGCCCAAGGATGTGCTCACCATTACTCTGACTCCTAAGGTC
ACGTGTGTTGTGGTAGACATCAGCAAGGATGATCCCGAGGTCCAGTTCAGC
TGGTTTGTAGATGATGTGGAGGTGCACACAGCTCAGACGCAACCCCGGGAG
GAGCAGTTCAACAGCACTTTCCGCTCAGTCAGTGAACTTCCCATCATGCAC
CAGGACTGGCTCAATGGCAAGGAGTTCAAATGCAGGGTCAACAGTGCAGCT
TTCCCTGCCCCCATCGAGAAAACCATCTCCAAAACCAAAGGCAGACCGAAG
GCTCCACAGGTGTACACCATTCCACCTCCCAAGGAGCAGATGGCCAAGGAT
AAAGTCAGTCTGACCTGCATGATAACAGACTTCTTCCCTGAAGACATTACT
GTGGAGTGGCAGTGGAATGGGCAGCCAGCGGAGAACTACAAGAACACTCAG
CCCATCATGGACACAGATGGCTCTTACTTCGTCTACAGCAAGCTCAATGTG
CAGAAGAGCAACTGGGAGGCAGGAAATACTTTCACCTGCTCTGTGTTACAT
GAGGGCCTGCACAACCACCATACTGAGAAGAGCCTCTCCCACTCTCCTGGT
AAA
Light Chain Variable Domain (VL) and CDR Amino
Acid and Nucleic Acid Sequences
(SEQ ID NO: 36)
DVLMTQTPLSLPVSLGDQASISCRSSQSIVNSNGNTYLEWYLQKPGQSPKL
LIYKVSNRFSGVPDRFSGSGSGTDFTLKISRVEAEDLGVYYCFQGSHVPLT
FGAGTKLELK

### Complementary Determining Region Sequences

| Scheme | CDR-L1 | CDR-L2 | CDR-L3 |
|---|---|---|---|
| Kabat | RSSQSIVNSNGNTYLE | KVSNRFS | FQGSHVPLT |
| | (SEQ ID NO: 37) | (SEQ ID NO: | (SEQ ID NO: 39) |
| | | 38) | |
| IMGT | QSIVNSNGNTY (SEQ | KVS | FQGSHVPLT (SEQ |
| | ID NO: 40) | | ID NO: 39) |
| Chothia | RSSQSIVNSNGNTYLE | KVSNRFS | FQGSHVPLT (SEQ |
| | (SEQ ID NO: 37) | (SEQ ID NO: | ID NO: 39) |
| | | 38) | |

(SEQ ID NO: 80)
GATGTTTTGATGACCCAAACTCCACTCTCCCTGCCTGTCAGTCTTGGAGAT
CAAGCCTCCATCTCTTGCAGATCTAGTCAGAGCATTGTAAATAGTAATGGA
AACACCTATTTAGAATGGTACCTGCAGAAACCAGGCCAGTCTCCAAAACTC
CTCATCTACAAAGTTTCCAACCGATTTTCTGGGGTCCCAGACAGGTTCAGT
GGCAGTGGATCAGGGACAGATTTCACACTCAAGATCAGCAGAGTGGAGGCT
GAGGATCTGGGAGTTTATTACTGCTTTCAAGGTTCACATGTTCCGCTCACG
TTCGGTGCTGGGACCAAGCTGGAGCTGAAA
Light Chain Constant Amino Acid and Nucleic Acid
Sequences
(SEQ ID NO: 20)
RADAAPTVSIFPPSSEQLTSGGASVVCFLNNFYPKDINVKWKIDGSERQNG
VLNSWTDQDSKDSTYSMSSTLTLTKDEYERHNSYTCEATHKTSTSPIVKSF
NRNEC
(SEQ ID NO: 21)
CGGGCTGATGCTGCACCAACTGTATCCATCTTCCCACCATCCAGTGAGCAG
TTAACATCTGGAGGTGCCTCAGTCGTGTGCTTCTTGAACAACTTCTACCCC
AAAGACATCAATGTCAAGTGGAAGATTGATGGCAGTGAACGACAAAATGGC
GTCCTGAACAGTTGGACTGATCAGGACAGCAAAGACAGCACCTACAGCATG
AGCAGCACCCTCACGTTGACCAAGGACGAGTATGAACGACATAACAGCTAT
ACCTGTGAGGCCACTCACAAGACATCAACTTCACCCATTGTCAAGAGCTTC
AACAGGAATGAGTGT

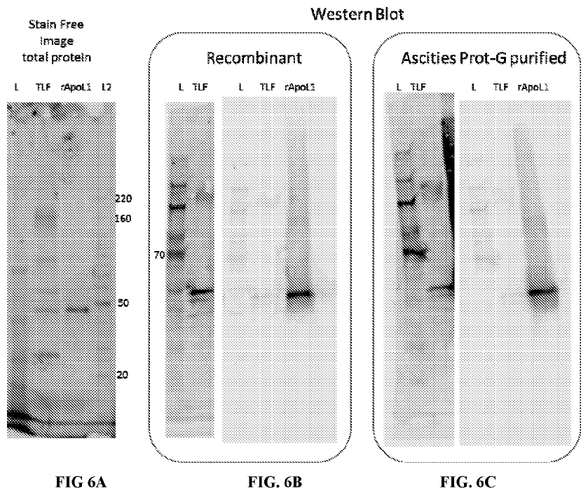

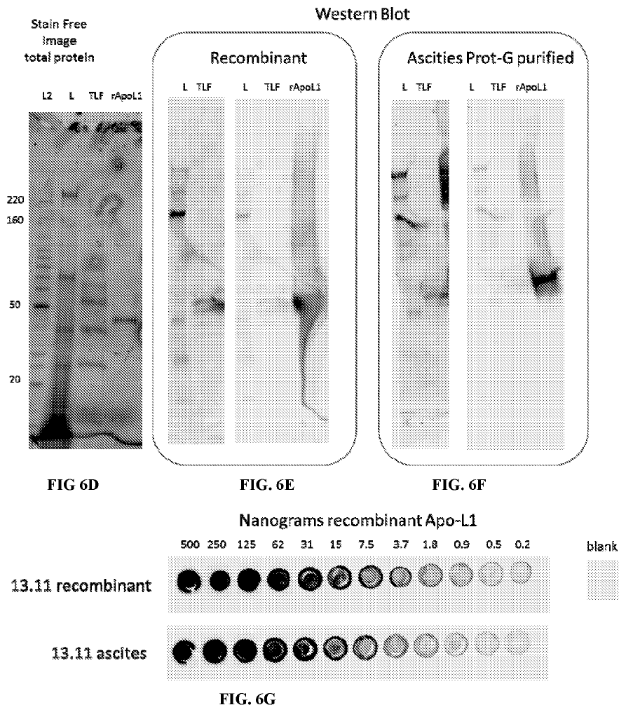

A Western blot assay comparing recombinant antibody and ascites Pro-G purified antibody, shows that both recombinant and purified antibodies bind in the TLF column and the rApoL1, under both non-reducing and reducing conditions. See FIGS. 6A-6F. Both antibodies bound recombinant ApoL1 in a Dot Blot (native) assay. See FIG. 6G.

### Example 7: Anti-BCMA scFv Binds BCMA

### Materials and Methods

17A5 scFv: The anti-BCMA clone 17A5 has the heavy and light chain variable sequences:

VH
(SEQ ID NO: 41)
EVQLLESGGGLVQPGGSLRLSCAASGFTFSSYAMSWVRQAPGKGLEWVSAI
SGSGGSTYYADSVKGRFTISRDNSKNTLYLQMNSLRAEDTAVYYCAKVAPY
FAPFDYWGQGTLVTVSS
VL
(SEQ ID NO: 42)
EIVLTQSPGTLSLSPGERATLSCRASQSVSSSYLAWYQQKPGQAPRLLIYG
ASSRATGIPDRFSGSGSGTDFTLTISRLEPEDFAVYYCQQYGNPPLYTFGQ
GTKVEIK

The CDR sequences of the anti-BCMA clone 17A5, bold in the sequences above are:

CDR1H:
(SEQ ID NO: 43)
SYAMS,
CDR2H:
(SEQ ID NO: 44)
AISGSGGSTYYADSVKG,
CDR3H:
(SEQ ID NO: 45)
VAPYFAPEDY,
CDRIL:
(SEQ ID NO: 46)
RASQSVSSSYLA,
CDR2L:
(SEQ ID NO: 47)
GASSRAT,
CDR3L:
(SEQ ID NO: 48)
QQYGNPPLYT.

The sequence of an anti-BCMA scFv of clone 17A5 (“17A5 scFv”) is EIVLTQSPGTLSLSPGERATLSCRASQSVSSSYLAWYQQKPGQAPRLLIYG ASSRATGIPDRFSGSGSGTDFTLTISRLEPEDFAVYYCQQYGNPPLYTFGQ GTKVEIKggggsggggsggggsEVQLLESGGGLVQPGGSLRLSCAASGFTF SSYAMSWVRQAPGKGLEWVSAISGSGGSTYYADSVKGRFTISRDNSKNTLY LQMNSLRAEDTAVYYCAKVAPYFAPFDYWGQGTLVTVSS (SEQ ID NO:51), where the CDRs are bold and ggggsggggsggggs (lowercase) (SEQ ID NO:52) is a flexible linker.

For BMCA clone 17A5 sequences, and alternative anti-BCMA sequences, see WO 2014/122144, which is specifically incorporated by reference herein in its entirety.
Materials: BCMA-BCMA Protein, Human, Recombinant (ECD, rFc Tag) (Sino biological 10620-H15H) (0.5 ug); L—Chameleon Duo Ladder (Licor) −8 uL; L2—Benchmark Protein Ladder (Thermo 10747012) −2 uL; Control BCMA monoclonal—anti-hBCMA, mouse monoclonal, Clone 1004023 (RnD Systems Cat. MAB1931)
Methods: SDS-Page 4%-15% Mini-PROTEAN TGX (200V, 30 min); Transferred to nitrocellulose (75V, 45 min); Block 1 hour in Intercept blocking buffer (PBS); 1:10,000 dilution of antibody in 0.5× Intercept blocking buffer (PBS)+0.2%; Tween−Overnight; Wash 4×PBS-T; 1:10,000 dilution of secondary antibody in 0.5× Intercept blocking buffer (PBS)+0.2% Tween−1 hour (Licor goat anti-mouse 800, 926-32210); Wash 4×PBS-T; Rinse 1×PBS; Image on Odyssey DLx Infrared Imager in the 800 channel

### Results

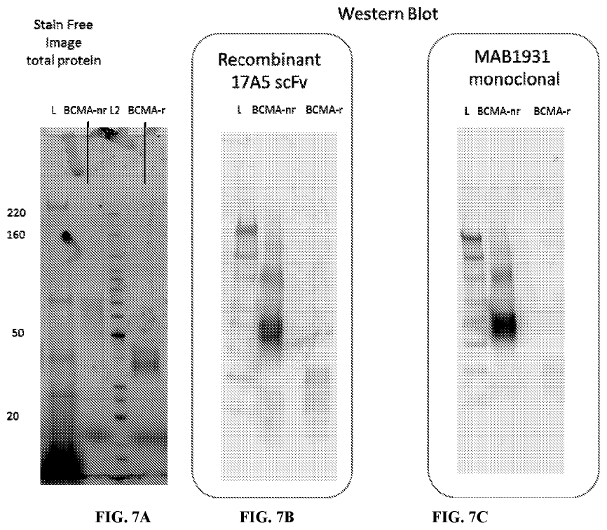

Anti-BCMA scFv of SEQ ID NO:51 was tested for its ability to bind recombinant BCMA in comparison to commercially available control monoclonal antibody (RnD Systems Cat. MAB1931) by Western blotting. scFv BCMA antibody binds to a similar mobility target—recombinant BCMA-verifying it specifically binds the correct target. See FIGS. 7A-7C.

### Example 8: Design of a Bispecific ApoL1-BCMA-bsAb Antibody

An anti-ApoL1, BCMA IgG1-scFv (Heavy Chain C-terminus) antibody has been designed and has the structure of FIG. 8.

The Fab portion is the heavy and light chain variable regions of recombinant clone SFIII 13.11 (anti-ApoL1) having the heavy chain variable domain of SEQ ID NO:24 and the light chain variable domain of SEQ ID NO:36 or SEQ ID NO:77.

The anti-BCMA is an ScFv of [clone 17A5] provided in Example 7 (SEQ ID NO:51), Human IgG1, Kappa fused to the Heavy Chain C-terminus of a human IgG1.

The complete Clone 13.11×BCMAscFv: bsAbBCMA/ApoL1 sequences used in the Examples that follow are:

Heavy Chain Amino Acid Sequence
(SEQ ID NO: 71)
EVQLVESGGGLVKPGGSLKLSCAASGFTESTYAMSWVRQSPEKRLEWVAEI
SNGGLYTYYPDTVTGRFTISRDNVKNILYLEMSSLRSEDTAIYYCIRENRN
WYFDLWGAGTTVTVSSASTKGPSVFPLAPSSKSTSGGTAALGCLVKDYFPE
PVTVSWNSGALTSGVHTFPAVLQSSGLYSLSSVVTVPSSSLGTQTYICNVN
HKPSNTKVDKKVEPKSCDKTHTCPPCPAPELLGGPSVFLFPPKPKDTLMIS
RTPEVTCVVVDVSHEDPEVKFNWYVDGVEVHNAKTKPREEQYNSTYRVVSV
LTVLHQDWLNGKEYKCKVSNKALPAPIEKTISKAKGQPREPQVYTLPPSRD
ELTKNQVSLTCLVKGFYPSDIAVEWESNGQPENNYKTTPPVLDSDGSFFLY
SKLTVDKSRWQQGNVFSCSVMHEALHNHYTQKSLSLSPGGGGSGGGGSGGG
GSEIVLTQSPGTLSLSPGERATLSCRASQSVSSSYLAWYQQKPGQAPRLLI
YGASSRATGIPDRFSGSGSGTDFTLTISRLEPEDFAVYYCQQYGNPPLYTF
GQGTKVEIKGGGGSGGGGSGGGGSEVQLLESGGGLVQPGGSLRLSCAASGF
TFSSYAMSWVRQAPGKGLEWVSAISGSGGSTYYADSVKGRFTISRDNSKNT
LYLQMNSLRAEDTAVYYCAKVAPYFAPFDYWGQGTLVTVSS
Heavy Chain Nucleic Acid Sequence
(SEQ ID NO: 73)
GAAGTGCAACTGGTGGAGTCCGGGGGGGGGTTAGTGAAACCCGGCGGATCT
CTGAAGTTGAGCTGCGCAGCTTCCGGCTTCACCTTCAGCACCTACGCCATG
AGCTGGGTCAGACAGAGCCCTGAGAAGAGACTGGAGTGGGTGGCCGAGATC
AGCAACGGAGGGCTGTACACATACTACCCCGACACCGTGACCGGAAGATTT
ACCATCTCTAGAGACAACGTTAAGAACATCCTGTACCTGGAGATGAGCAGC
CTGAGAAGCGAAGACACCGCCATCTACTACTGCATCAGAGAGAACAGAAAC
TGGTACTTCGACCTGTGGGGGGCCGGCACCACAGTGACTGTGAGTTCCGCA
TCCACCAAGGGTCCCTCAGTATTCCCCCTGGCTCCTAGCTCCAAGAGCACG
AGCGGGGGCACTGCCGCACTGGGCTGTCTCGTCAAGGACTACTTTCCTGAG
CCTGTGACAGTGAGCTGGAACAGCGGGGCCCTGACAAGCGGCGTCCACACC
TTCCCCGCCGTGCTGCAGAGCAGCGGCCTATATTCACTTAGCAGCGTGGTG
ACCGTGCCTAGCAGCAGCCTGGGCACACAGACCTACATCTGCAACGTGAAC
CACAAGCCTAGCAACACCAAGGTGGACAAGAAGGTGGAGCCGAAGAGCTGT
GACAAGACGCATACGTGCCCTCCCTGCCCCGCACCCGAACTGTTGGGCGGG
CCCTCAGTGTTCCTGTTCCCCCCCAAGCCCAAGGACACCCTGATGATCAGC
AGAACCCCTGAGGTGACCTGCGTGGTGGTGGACGTGAGCCACGAGGACCCT
GAGGTGAAGTTCAACTGGTACGTGGACGGCGTGGAGGTGCACAACGCCAAG
ACCAAGCCTAGAGAGGAGCAGTACAACAGCACCTACAGAGTGGTGAGCGTG
CTGACCGTGCTGCACCAAGACTGGCTGAACGGCAAGGAGTACAAGTGCAAG
GTGAGCAACAAGGCCCTGCCCGCCCCCATCGAGAAGACCATCAGCAAGGCC
AAGGGGCAGCCTAGAGAGCCCCAAGTGTACACCCTGCCCCCTAGCAGAGAC
GAGCTGACCAAGAACCAAGTGAGCCTGACCTGTCTGGTGAAGGGTTTCTAC
CCTAGCGACATCGCCGTGGAGTGGGAGAGCAACGGGCAGCCTGAGAACAAC
TACAAGACCACCCCCCCCGTGCTGGACAGCGACGGCAGCTTCTTCCTGTAC
AGCAAGCTGACCGTGGACAAGAGCAGATGGCAGCAAGGCAACGTGTTCAGC
TGCAGCGTGATGCACGAGGCCCTGCACAACCATTACACGCAGAAGTCACTG
TCGCTGTCTCCCGGCGGTGGCGGATCTGGGGGGGGTGGTTCTGGGGGTGGG
GGTTCCGAAATCGTCCTGACACAAAGTCCCGGCACACTGTCTCTGTCTCCC
GGCGAAAGAGCCACCCTGAGCTGCAGAGCATCTCAGAGCGTGAGCAGCAGC
TACCTGGCCTGGTATCAGCAGAAGCCCGGCCAAGCCCCTAGACTGCTGATC
TACGGCGCAAGCAGCAGAGCCACCGGCATCCCCGACAGATTCAGCGGCAGC
GGCTCTGGCACCGACTTCACCCTGACCATCTCTCGACTGGAGCCTGAGGAC
TTCGCCGTGTACTATTGCCAACAGTACGGCAACCCCCCCCTGTACACCTTC
GGCCAAGGAACCAAAGTCGAGATCAAGGGGGGCGGAGGGAGCGGCGGAGGC
GGAAGCGGCGGTGGTGGTTCGGAGGTACAACTATTGGAATCCGGTGGCGGA
TTAGTGCAGCCGGGCGGCAGCTTAAGACTCTCATGCGCCGCGTCGGGCTTT
ACCTTTAGTAGCTATGCCATGTCCTGGGTTCGGCAAGCCCCCGGCAAAGGC
CTGGAGTGGGTGTCAGCGATCAGCGGCAGCGGAGGTAGCACCTACTACGCC
GACAGCGTGAAGGGTAGATTCACGATCAGCAGAGACAATAGCAAGAACACC
CTGTACCTGCAGATGAACAGCCTGAGAGCCGAGGACACTGCCGTATACTAC
TGCGCCAAGGTGGCCCCCTACTTCGCCCCCTTCGACTACTGGGGCCAAGGC
ACACTGGTCACCGTCAGCTCG
Light Chain Amino Acid Sequence
(SEQ ID NO: 72)
DVLMTQTPLSLPVSLGDQASISCRSSQSIVNSNGNTYLEWYLQKPGQSPKL
LIYKVSNRFSGVPDRFSGSGSGTDFTLKISRVEAEDLGVYYCFQGSHVPLT
FGAGTKLEIKRTVAAPSVFIFPPSDEQLKSGTASVVCLLNNFYPREAKVQW
KVDNALQSGNSQESVTEQDSKDSTYSLSSTLTLSKADYEKHKVYACEVTHQ
GLSSPVTKSFNRGEC
Light Chain Nucleic Acid Sequence
(SEQ ID NO: 74)
GACGTGCTGATGACACAGACCCCCCTGAGCCTGCCCGTGAGCCTGGGCGAC
CAAGCAAGCATCAGCTGCAGAAGCTCTCAGAGCATCGTGAACAGCAACGGC
AACACCTACCTGGAGTGGTACCTGCAGAAGCCTGGGCAGAGCCCCAAGCTG
CTGATCTACAAGGTGAGCAATCGGTTCAGTGGGGTGCCTGACAGATTCAGT
GGTTCCGGTAGCGGCACCGACTTCACCCTGAAGATCAGCAGAGTGGAGGCC
GAGGACCTGGGCGTGTACTACTGCTTCCAAGGCAGCCACGTGCCCCTGACC
TTCGGGGCCGGCACCAAGCTCGAGATCAAGAGAACTGTGGCCGCGCCGTCA
GTGTTTATCTTCCCTCCATCGGATGAACAGCTTAAGTCCGGCACGGCGTCT
GTGGTCTGCCTGCTCAATAACTTTTACCCTAGGGAAGCTAAAGTCCAATGG
AAAGTGGATAACGCCCTGCAGTCAGGAAACAGCCAGGAATCGGTTACCGAA
CAGGACAGCAAGGACAGCACTTACTCCTTGTCGTCGACTCTTACTCTGAGC
AAGGCCGATTACGAGAAGCACAAGGTCTACGCCTGCGAGGTCACCCATCAG
GGACTCTCGTCCCCGGTGACCAAATCCTTCAATAGAGGCGAATGC

### Example 9: Bispecific ApoL1-BCMA-bsAb antibody binds BCMA and

### Apolipoprotein L1

### Materials and Methods

### ELISA

### Immobilization and Blocking

Enzyme-Linked Immunosorbent Assay (ELISA) plates were prepared by incubating them overnight at 4° C. with their respective immobilization buffer (bicarbonate/carbonate coating buffer 100 mM) or blocking buffer (PBS, 0.1% Tween-20, 1% BSA).

### Bridging ELISA

For the bridging ELISA experiments, ApoL1 (Sino Cat.13910-H08B) or BCMA (Sino Cat. 10620-H15H) were resuspended at a 1:10,000 dilution in ELISA binding buffer (1×PBST+1% BSA) and then incubated overnight at 4° C. ApoL1 was immobilized across all conditions except where BCMA was immobilized as a primary protein (ApoL1-*). Following immobilization, 100 μl of bispecific antibody (bsAb, 1 g/ml in ELISA blocking buffer) was added and allowed to incubate for 1 hour at 37° C. Subsequently, each well underwent four rounds of 150 μl washes with 1×PBST. Next, 1 μg/mL rabbit Fc tagged BCMA in ELISA blocking buffer was allowed to incubate for 1 hr at 37° C. followed by four more rounds of 150 μl 1×PBST washes. A secondary binding component, anti-rabbit HRP, was added at 1:10,000 and the plate was incubated for an additional hour at 37° C. followed by another four rounds of 150 μl washes with 1×PBST. To visualize the reaction, TMB One-Step Substrate Reagent (Sigma) was applied in a 50 μl volume and allowed to incubate for no more than 15 minutes. The reaction was halted by adding 50 μl of Stop Solution, and the absorbance was promptly measured at 450 nm on a SpectraMax iD3 plate reader (Molecular Diagnostics).

### ELISA with Normal Human Serum

For ELISA involving normal human serum (NHS), conditioned media was combined with ELISA binding buffer in a 1:1 ratio. This mixture was incubated at 4° C. overnight, followed by an additional overnight incubation with blocking buffer at 4° C. (PBS, 1% BSA, 0.1% Tween-20). Subsequently, the prepared samples were exposed to an HRP-labeled bsAb, introduced at a 1:1,000 dilution (1p g/mL final), and allowed to incubate for 1 hour at 37° C. Afterward, each well underwent four 150 μl washes with 1×PBST. Lastly, TMB One-Step Substrate Reagent (Sigma) was added in a 50 μl volume, with a 15-minute incubation period. The reaction was terminated by adding 50 μl of Stop Solution, and absorbance was immediately read at 450 nm on a SpectraMax iD3 plate reader (Molecular Diagnostics).

### Bispecific Antibody Competition ELISA

A competition ELISA was conducted to investigate the competitive binding interactions involving B-Cell Maturation Antigen (BCMA) and the bsAb. BCMA protein was immobilized onto ELISA plate wells at a dilution of 1:10,000, with each well receiving 0.1 μg of the immobilized protein for 1 hour at 37° C. During the incubation, the competition was set by combining HRP-labeled-bsAb at a dilution of 1:10,000, at a concentration of 0.1p g per well, with unlabeled bsAb in varying concentrations, ranging from 0 to 100 times the original concentration (max at 10 g per well). The two forms of bsAb's were combined, added to the ELISA plate, and allowed to incubate for 1 hour at 37° C. Afterward, each well underwent four 150 μl washes with 1×PBST. Lastly, TMB One-Step Substrate Reagent (Sigma) was added in a 50 μl volume, with a 5-minute incubation period. The reaction was terminated by adding 50 μl of Stop Solution, and absorbance was immediately read at 450 nm on a SpectraMax iD3 plate reader (Molecular Diagnostics).

### Results

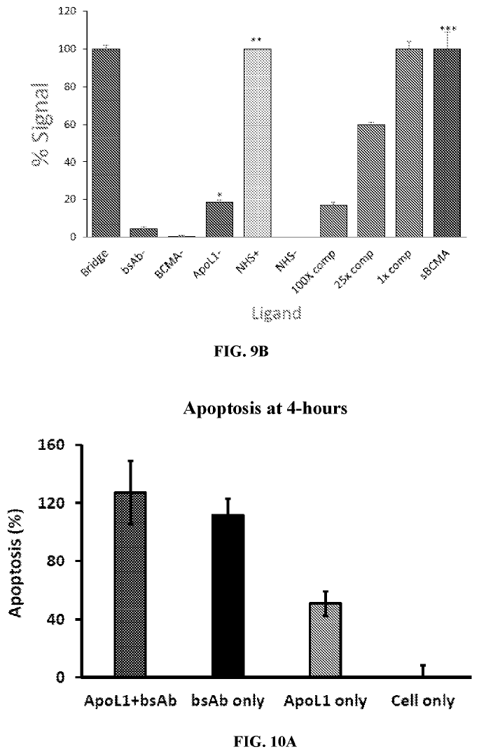

Experiments were designed to test the binding of the bispecific ApoL1-BCMA-bsAb antibody of Example 8. Results show the bispecific antibody effectively engages with its designated targets, ApoL1 and BCMA, encompassing both native and recombinant iterations in a dose dependent manner. The bsAb's specificity towards these targets is substantiated by its capacity to precisely out-compete HRP-labeled bsAb in a competition-bridging ELISA. As discussed in more detail below, this data displays the precision and selectivity of the bsAb in engaging with its designated targets (FIGS. 9A-9B).

A bridging ELISA was used to assess the binding capacity of the bispecific antibody (bsAb) between recombinant variations of ApoL1 and BCMA (FIG. 9A). The bsAb demonstrated successful binding to immobilized forms of both ligands (Bridge). Conversely, no detectable signal was recorded when bsAb, ApoL1, or BCMA were absent (bsAb−, BCMA−, ApoL1−).

Native ApoL1 present in normal human serum exhibited similar binding affinity with the bsAb (NHS+), as did soluble BCMA released from RPMI 8226 Multiple Myeloma cells (sBCMA). Variations in immobilized factors on the ELISA plates: *recombinant BCMA, **Normal human serum, ***Conditioned media from CCL-155 cells on day 4. Bar graphs display the mean of the percentage of signal as compared to the mean of the full bridge sample. Data represents the means after three replicates with error bars depicting standard deviation (FIG. 9B).

### Example 10: a Bispecific ApoL1-BCMA-bsAb Antibody Facilitates an Increase in ApoL1-Induced Cell Death Processes

### Materials and Methods

### Apoptosis and Necrosis Assays

Cells were cultivated in RPMI-1640 with 10% FBS, 10 nM LY411575, and 100 μg/mL human IgG1. Cells were inoculated into a 96 well, black clear bottom plate (Greiner Cat. 07-000-166) at 1×10∝cells/well (2×10≡cells/ml) and incubated with 3.1 μg/mL ApoL1, 15 μg/mL bsAb, and Promega RealTime-Glo Annexin V Apoptosis and Necrosis reagents (Promega Cat. JA1011). Plates were incubated at 37° C., 5% CO2 for 20 hours and measured every four hours. Measurements taken at each timepoint were gathered on a SpectraMax iD3 plate reader (Molecular Diagnostics). Apoptosis was measured via luminescence top-read detector and Necrosis was measured under fluorescence with an excitation of 485 nm and emission measured at 525 nm. Data was imported into GraphPad, and standard error and t-test performed to measure β-values indicated. Data was normalized by setting baseline to Cell only, then expressing data as an average mean percentage difference compared to the baseline.

### Results

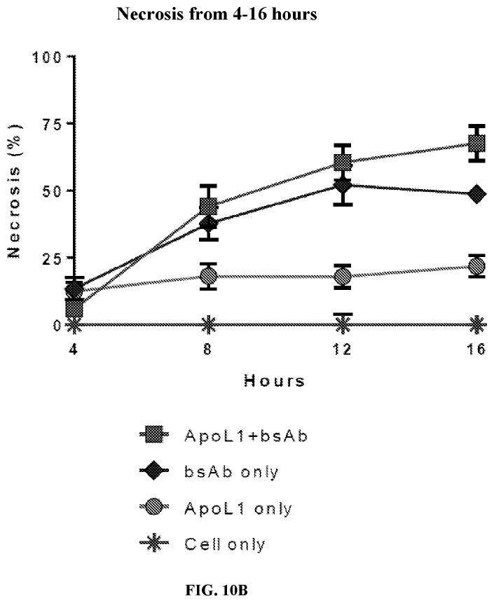

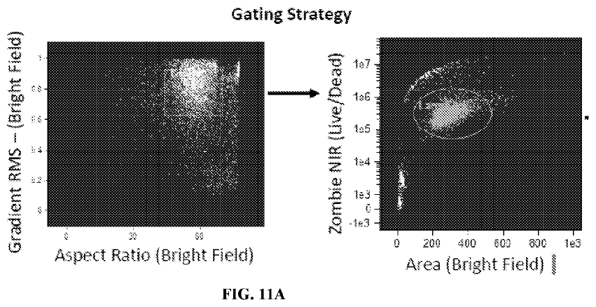

Experiments were designed to determine if bispecific antibody could increase cell death in target cells. Cells were treated with the bispecific antibody of Example 8 alone, ApoL1 alone, or a combination of antibody and ApoL1. Results indicates that introducing the bsAb to RPMI 8226 cells leads to a rise in ApoL1-induced apoptosis within four hours after application, and an increase in ApoL1-induced necrosis between 4-16 hours following incubation. The extension of apoptosis is recorded at 127% when both ApoL1 and bsAb are co-administered, 111% when bsAb is administered singularly thereby relying on endogenous ApoL1 in culture, and 50% when additional ApoL1 is individually introduced (FIG. 10A).

At 16 hours cells treated with both ApoL1 and bsAb exhibit a 67% rise in necrosis compared to cells treated with bsAb alone. Individually, ApoL1 and bsAb display increases in necrosis by 27% and 49%, respectively (FIG. 10A).

Collectively, these findings show that the introduction of bsAb alone is adequate to stimulate apoptotic and necrotic signals within Multiple Myeloma beyond the levels achievable with ApoL1 alone, and that endogenous ApoL1 is present at sufficient levels to be utilized by the bsAb for the induction of both apoptosis and necrosis.

### Example 11: ApoL1-BCMA-bsAb and ApoL1-488 can Bind to RPMI8226 (CCL-155) Multiple Myeloma Cells

### Materials and Methods

### Flow Cytometry

### Antibodies, Proteins, and Antibody Labeling

Proteins and antibodies used are Recombinant ApoL1 (Sino Biological Cat. 3910-H08B), Ultra-Leaf Human IgG isotype control (BioLegend Cat. 403502) and ApoL1-BCMA bsAb (custom). All antibodies and crosslinked proteins used in these experiments were Alexa labeled using Alexa Fluor 488 Conjugation Kit—Lightning-Link (ab236553) according to manufacturer directions.

### Cell Preparation

Cells were cultured according to standard protocols. All cell pelleting were performed at 300× g for 5 minutes at 4 C. One to two days prior to the experiment, cells were washed and split to achieve a cell density of approximately 2-4×10≡cells/mL in a total volume of 10-30 mL containing 10 nM LY411575 (LY). On the day of the experiment, Zombie NIR dye (BioLegend Cat. 423106) was diluted 1:100 in phosphate-buffered saline (PBS). Cells were washed and resuspended in PBS with Zombie NIR dye at a concentration of 1×10\hat{ }7 cells/mL and incubated for 10 minutes at room temperature. After the incubation, cells were washed with PBS and the cell pellet was resuspended in equal volume of 4% paraformaldehyde (PFA) and incubated for 10 minutes at RT. The fixed cells were washed 2× with Cell Stain Buffer (CSB) (dPBS, 2% FBS, 0.1% NaN3), with a final resuspension in Cell Blocking Buffer (CBB) containing dPBS, pH 7.4, 2% FBS, 0.1% NaN3, and 100 g/mL human IgG Isotype Control (Fisher Cat. 31154) and incubated overnight.

### Antibody Incubation

On the following day, cells were split to 50l aliquots for each reaction, and antibody was added at the indicated concentrations. The antibody incubation was carried out for 30 minutes to 1.5 hours, on ice in the dark. After antibody incubation, cells were washed twice in CSB and resuspended in 50 pL of ice-cold (CSB).

### ApoL1-488 Binding

Cells were prepared as above with the addition of 15 μg/mL unlabeled ApoL1 in the blocking buffer for overnight incubation. After blocking, cells were washed 2 times with CSB and aliquoted to 50 μl aliquots for each reaction. First, bsAb or isotype control was added at 30 μg/mL for 1.5 hours, followed by 2 washes and 1 μg/nL of Alexa labeled ApoL1-488. Cells were incubated for 1 hour and washed 3 times with the final wash resuspended in 50 μl of CSB.

### Imaging Flow Cytometry

Imaging flow cytometry acquisition was performed on an ImageStream X MK II (Luminex Corporation, Seattle, Washington) with fluidics set at low speed, sensitivity set to high, magnification set to 60×, and illumination set to: 405:120 mW, 488:200 mW, 561:200 mW, 642:150 mW, SSC:4.38 mW. All samples were acquired using the autosampler with INSPIRE software for an acquisition time of 4000 events or 8 minutes per sample, with events counting SSC channel from 5.2×10\hat{ }5 to 8.9×10\hat{ }7. Gates were set and quantified using IDEAS software version 6.2.

### Results

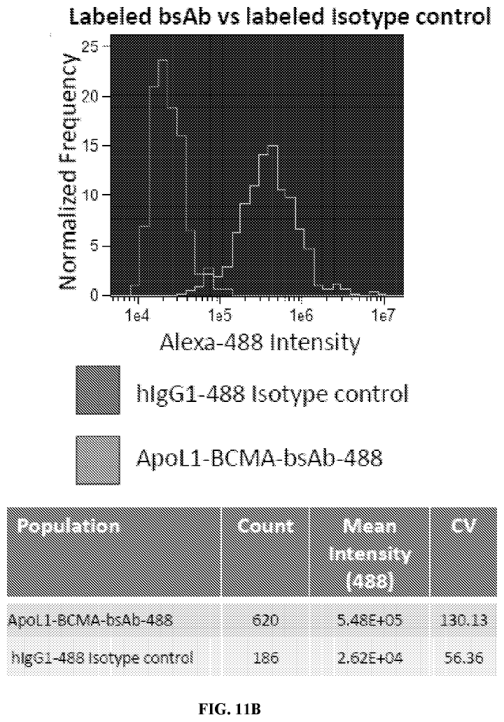

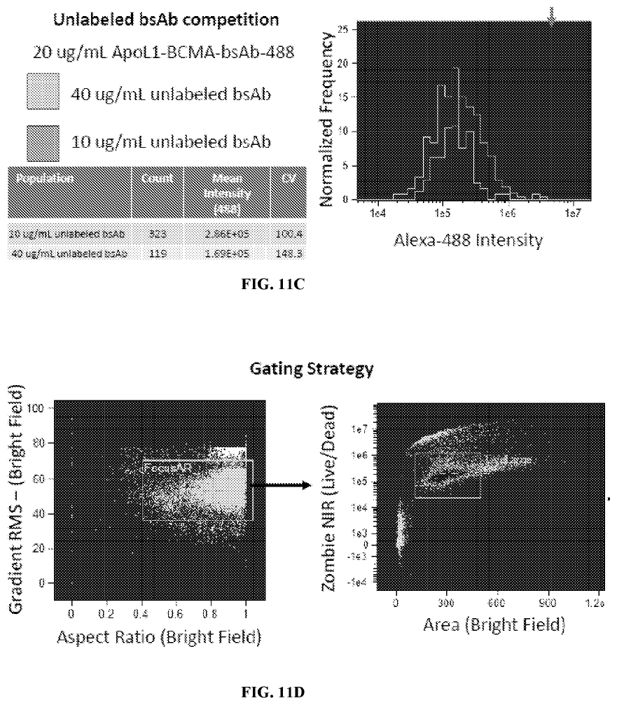

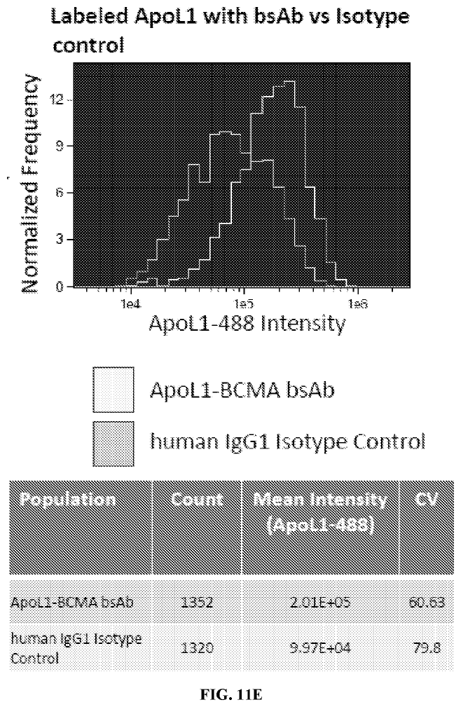

Imaging flow cytometry was used as an assay to determine if the ApoL1-BCMA-bsAb (bsAb) of Example 8 binds the two intended targets in vitro. First, the bsAb and a human IgG1 isotype control were Alexa-488 labeled and binding affinities were determined by measuring total cellular florescence after incubation. At 3 μg/mL, the bsAb shows a 20× increase of measured florescence per cell when compared to isotype control (FIG. 11A). Additionally, when unlabeled antibody was added as a competitive inhibitor, a 60% reduction in average total florescence per cell dependent on the molar ratio of unlabeled to labeled antibody (unlabeled: labeled of 1:2 vs 2:1) was observed (FIG. 11B).

Next, Alexa-488 labeled recombinant ApoL1 was used to determine if cell bound bsAb can bind ApoL1. Some background binding of labeled ApoL1 to cells without bsAb, likely due to existing binding affinities for ApoL1 to cell membranes, was foreseen. With bsAb added, however, a 2× increase in average cell florescence was observed when compared to addition of isotype control at the concentrations given (FIG. 11E).

Collectively, these results demonstrated that anti-ApoL1 portion of the bispecific antibody targets TLF and the anti-BCMA portion of the bispecific antibody targets MM cells, ultimately increasing internalization of TLF in the MM cells, and inducing cell death as illustrated in FIG. 12.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize or be able to ascertain using no more than routine experimentation, many equivalents to the specific embodiments of the invention described herein. Such equivalents are intended to be encompassed by the following claims.

We claim:

1. A method of increasing cell death of target cells in a mammalian subject in need thereof comprising administering the subject an effective amount of a composition that increases Apolipoprotein L1 (ApoL1) in the target cells.

2. The method of claim 1, wherein the composition increases accumulation of endogenous ApoL1 in the target cells.

3. The method of claim 2, wherein the endogenous ApoL1 is a component of an ApoL1-containing complex.

4. The method of claim 3, wherein the ApoL1-containing complex is a Trypanosome Lytic Factor (TLF), optionally TLF-1 and/or TLF-2.

5. The method of any one of claims 1-4, wherein the composition comprises a first antigen binding fragment that binds to ApoL1 or an ApoL1-containing complex and a targeting moiety that targets the composition to target cells, optionally wherein the composition is a bi- or multispecific antibody a first antigen binding fragment that binds to ApoL1 or an ApoL1-containing complex optionally a TLF and a second antigen binding fragment that binds to a cell specific antigen.

6. The method of claim 1, wherein the composition comprises a ApoL1 or a function fragment or variant thereof and a targeting moiety for a cell specific antigen.

7. The method of claim 6, wherein the composition comprises the ApoL1 or a function fragment or variant thereof conjugated or fused directly or indirectly the targeting moiety.

8. The method of claims 6 or 7, wherein the composition comprises a delivery vehicle, optionally liposomes or polymeric nanoparticles.

9. The method of claim 8, wherein the targeting moiety is conjugated or fused to the delivery vehicle.

10. The method of any one of claims 6-9, wherein the targeting moiety is an antibody or antigen bind fragment.

11. The method of any one of claims 1-10, wherein the cell specific antigen is specific for diseased cells.

12. The method of claim 11, wherein the diseased cells are cancer cells.

13. The method of claim 12, wherein the cancer cells are blood cancer cells.

14. The method of any one of claims 1-13, where the subject suffers from a disease caused by the target cells.

15. The method of claim 14, wherein the composition is administered in an effective amount to treat the disease.

16. The method of any one of claims 1-15, wherein the cell specific antigen is not a trypanosome specific surface antigen.

17. The method of any one of claims 1-16, wherein the subject does not have trypanosomiasis.

18. A composition comprising ApoL1 or a function fragment or variant thereof and a targeting moiety, wherein the targeting moiety does not target a trypanosome specific surface antigen.

19. The composition of claim 18, wherein the ApoL1 or a function fragment or variant thereof conjugated or fused directly or indirectly the targeting moiety.

20. The composition of claim 19, wherein the composition comprises a delivery vehicle, optionally liposomes or polymeric nanoparticles, optionally, wherein the targeting moiety is conjugated or fused to the delivery vehicle.

21. An antibody or antigen binding fragment comprising:
the three complementarity determining regions (CDRs) of the heavy chain variable domain of SEQ ID NO:24, or variants or humanized forms thereof with at least 70, 80, 90, or 95% sequence identity thereto, and the three complementarity determining regions (CDRs) of the light chain variable domain of SEQ ID NO:36 or SEQ ID NO:77, or variants or humanized forms thereof with at least 70, 80, 90, or 95% sequence identity thereto,
wherein the antibody or antigen binding fragment binds to Apolipoprotein L1 (ApoL1).

22. The antibody or antigen binding fragment of claim 21, wherein the heavy and light chain variable domain CDRs comprise:
(SEQ ID NO: 25)
TYAMS,
(SEQ ID NO: 26)
EISNGGLYTYYPDTVTG,
(SEQ ID NO: 27)
ENRNWYFDL,
(SEQ ID NO: 37)
RSSQSIVNSNGNTYLE,
and
(SEQ ID NO: 38)
KVSNRFS,
(SEQ ID NO: 39)
FQGSHVPLT,
or
variants or humanized forms thereof with at
least 70, 80, 90, or 95% sequence identity
thereto;
(SEQ ID NO: 28)
GFTFSTYA,
(SEQ ID NO: 29)
ISNGGLYT,
(SEQ ID NO: 30)
IRENRNWYFDL,
(SEQ ID NO: 40)
QSIVNSNGNTY,
KVS,
and
(SEQ ID NO: 39)
FQGSHVPLT,
or
variants or humanized forms thereof with at least
70, 80, 90, or 95% sequence identity thereto;
or
(SEQ ID NO: 31)
GFTFSTY,
(SEQ ID NO: 32)
SNGGLY,
(SEQ ID NO: 27)
ENRNWYFDL,
(SEQ ID NO: 37)
RSSQSIVNSNGNTYLE,
(SEQ ID NO: 38)
KVSNRFS,
and
(SEQ ID NO: 39)
FQGSHVPLT,
or
variants or humanized forms thereof with at least
70, 80, 90, or 95% sequence identity thereto.

23. The antibody or antigen binding fragment of claims 21 or 22, wherein the heavy and light chain variable domain CDRs comprise:
(SEQ ID NO: 25)
TYAMS,
(SEQ ID NO: 26)
EISNGGLYTYYPDTVTG,
(SEQ ID NO: 27)
ENRNWYFDL,
(SEQ ID NO: 37)
RSSQSIVNSNGNTYLE,
and
(SEQ ID NO: 38)
KVSNRFS,
(SEQ ID NO: 39)
FQGSHVPLT;
(SEQ ID NO: 28)
GFTFSTYA,
(SEQ ID NO: 29)
ISNGGLYT,
(SEQ ID NO: 30)
IRENRNWYFDL,
(SEQ ID NO: 40)
QSIVNSNGNTY,
KVS,
and
(SEQ ID NO: 39)
FQGSHVPLT;
or
(SEQ ID NO: 31)
GFTFSTY,
(SEQ ID NO: 32)
SNGGLY,
(SEQ ID NO: 27)
ENRNWYFDL,
(SEQ ID NO: 37)
RSSQSIVNSNGNTYLE,
(SEQ ID NO: 38)
KVSNRFS,
and
(SEQ ID NO: 39)
FQGSHVPLT.

24. The antibody or antigen binding fragment of any one of claims 21-23 comprising a heavy chain variable domain comprising the amino acid sequence of SEQ ID NO:24 or a variant or humanized form thereof with at least 70, 80, 90, or 95% sequence identity thereto and a light chain variable domain comprising the amino acid sequence of SEQ ID NO:36 or SEQ ID NO:77 or a variant or humanized form thereof with at least 70, 80, 90, or 95% sequence identity thereto.

25. The antibody or antigen binding fragment thereof of any one of claims 21-24 comprising a heavy chain variable domain comprising the amino acid sequence of SEQ ID NO:24 and a light chain variable domain comprising the amino acid sequence of SEQ ID NO:36 or SEQ ID NO:77.

26. An antibody or antigen binding fragment comprising:
the three complementarity determining regions (CDRs) of the heavy chain variable domain of SEQ ID NO:3, or variants or humanized forms thereof with at least 70, 80, 90, or 95% sequence identity thereto, and the three complementarity determining regions (CDRs) of the light chain variable domain of SEQ ID NO:14, or variants or humanized forms thereof with at least 70, 80, 90, or 95% sequence identity thereto,
wherein the antibody or antigen binding fragment binds to Haptoglobin related protein (Hpr).

27. The antibody or antigen binding fragment of claim 26, wherein the heavy and light chain variable domain CDRs comprise:
(SEQ ID NO: 4)
NYGMN,
(SEQ ID NO: 5)
WINSYTGEATYTDDLKG,
(SEQ ID NO: 6)
EGYGDYGYSFDY,
(SEQ ID NO: 16)
RATKNIYTYLA,
(SEQ ID NO: 17)
NAKTLAE,
and
(SEQ ID NO: 18)
QHHYGTPRT,
or
variants or humanized forms thereof with at least
70, 80, 90, or 95% sequence identity thereto;
(SEQ ID NO: 7)
GYIFTNYG,
(SEQ ID NO: 8)
INSYTGEA,
(SEQ ID NO: 9)
AREGYGDYGYSFDY,
(SEQ ID NO: 19)
KNIYTY,
NAK,
and
(SEQ ID NO: 18)
QHHYGTPRT,
or
variants or humanized forms thereof with at least
70, 80, 90, or 95% sequence identity thereto;
or
(SEQ ID NO: 10)
GYIFTNY,
(SEQ ID NO: 11)
NSYTGE,
(SEQ ID NO: 6)
EGYGDYGYSFDY,
(SEQ ID NO: 16)
RATKNIYTYLA,
(SEQ ID NO: 17)
NAKTLAE,
and
(SEQ ID NO: 18)
QHHYGTPRT,
or
variants or humanized forms thereof with at least
70, 80, 90, or 95% sequence identity thereto.

28. The antibody or antigen binding fragment of claims 26 or 27, wherein the heavy and light chain variable domain CDRs comprise:
(SEQ ID NO: 4)
NYGMN,
(SEQ ID NO: 5)
WINSYTGEATYTDDLKG,
(SEQ ID NO: 6)
EGYGDYGYSFDY,
(SEQ ID NO: 16)
RATKNIYTYLA,
(SEQ ID NO: 17)
NAKTLAE,
and
(SEQ ID NO: 18)
QHHYGTPRT;
(SEQ ID NO: 7)
GYIFTNYG,
(SEQ ID NO: 8)
INSYTGEA,
(SEQ ID NO: 9)
AREGYGDYGYSFDY,
(SEQ ID NO: 19)
KNIYTY,
NAK,
and
(SEQ ID NO: 18)
QHHYGTPRT;
or
(SEQ ID NO: 10)
GYIFTNY,
(SEQ ID NO: 11)
NSYTGE,
(SEQ ID NO: 6)
EGYGDYGYSFDY,
(SEQ ID NO: 16)
RATKNIYTYLA,
(SEQ ID NO: 17)
NAKTLAE,
and
(SEQ ID NO: 18)
QHHYGTPRT.

29. The antibody or antigen binding fragment of any one of claims 26-28 comprising a heavy chain variable domain comprising the amino acid sequence of SEQ ID NO:3 or a variant or humanized form thereof with at least 70, 80, 90, or 95% sequence identity thereto and a light chain variable domain comprising the amino acid sequence of SEQ ID NO:14 or a variant or humanized form thereof with at least 70, 80, 90, or 95% sequence identity thereto.

30. The antibody or antigen binding fragment thereof of any one of claims 26-29 comprising a heavy chain variable domain comprising the amino acid sequence of SEQ ID NO:3 and a light chain variable domain comprising the amino acid sequence of SEQ ID NO:14.

31. An antibody or antigen binding fragment comprising:
the three complementarity determining regions (CDRs) of the heavy chain variable domain of SEQ ID NO:56, or variants or humanized forms thereof with at least 70, 80, 90, or 95% sequence identity thereto, and the three complementarity determining regions (CDRs) of the light chain variable domain of SEQ ID NO:65, or variants or humanized forms thereof with at least 70, 80, 90, or 95% sequence identity thereto,
wherein the antibody or antigen binding fragment binds to Haptoglobin related protein (Hpr).

32. The antibody or antigen binding fragment of claim 31, wherein the heavy and light chain variable domain CDRs comprise:
(SEQ ID NO: 57)
DYSIH,
(SEQ ID NO: 58)
WKHTESGESTYADDEKG,
(SEQ ID NO: 59)
GANYGSLLDY,
(SEQ ID NO: 66)
RASKSVSTSGYSYMH,
(SEQ ID NO: 67)
LASNLES,
(SEQ ID NO: 68)
QHNRELPLT,
or
variants or humanized forms thereof with at least
70, 80, 90, or 95% sequence identity thereto;
(SEQ ID NO: 60)
GFTFTDYS,
(SEQ ID NO: 61)
KHTESGES,
(SEQ ID NO: 62)
ARGANYGSLLDY,
(SEQ ID NO: 69)
KSVSTSGYSY,
LAS,
(SEQ ID NO: 68)
QHNRELPLT,
or
variants or humanized forms thereof with at least
70, 80, 90, or 95% sequence identity thereto;
or
(SEQ ID NO: 63)
GFTFTDY,
(SEQ ID NO: 64)
HTESGE,
(SEQ ID NO: 59)
GANYGSLLDY,
(SEQ ID NO: 66)
RASKSVSTSGYSYMH,
(SEQ ID NO: 67)
LASNLES,
(SEQ ID NO: 68)
QHNRELPLT,
or
variants or humanized forms thereof with at least
70, 80, 90, or 95% sequence identity thereto.

33. The antibody or antigen binding fragment of claims 31 or 32, wherein the heavy and light chain variable domain CDRs comprise:
(SEQ ID NO: 57)
DYSIH,
(SEQ ID NO: 58)
WKHTESGESTYADDEKG,
(SEQ ID NO: 59)
GANYGSLLDY,
(SEQ ID NO: 66)
RASKSVSTSGYSYMH,
(SEQ ID NO: 67)
LASNLES,
(SEQ ID NO: 68)
QHNRELPLT;
(SEQ ID NO: 60)
GFTFTDYS,
(SEQ ID NO: 61)
KHTESGES,
(SEQ ID NO: 62)
ARGANYGSLLDY,
(SEQ ID NO: 69)
KSVSTSGYSY,
LAS,
(SEQ ID NO: 68)
QHNRELPLT;
or
(SEQ ID NO: 63)
GFTFTDY,
(SEQ ID NO: 64)
HTESGE,
(SEQ ID NO: 59)
GANYGSLLDY,
(SEQ ID NO: 66)
RASKSVSTSGYSYMH,
(SEQ ID NO: 67)
LASNLES,
(SEQ ID NO: 68)
QHNRELPLT.

34. The antibody or antigen binding fragment of any one of claims 31-33 comprising a heavy chain variable domain comprising the amino acid sequence of SEQ ID NO:56 or a variant or humanized form thereof with at least 70, 80, 90, or 95% sequence identity thereto and a light chain variable domain comprising the amino acid sequence of SEQ ID NO:65 or a variant or humanized form thereof with at least 70, 80, 90, or 95% sequence identity thereto.

35. The antibody or antigen binding fragment thereof of any one of claims 31-34 comprising a heavy chain variable domain comprising the amino acid sequence of SEQ ID NO:56 and a light chain variable domain comprising the amino acid sequence of SEQ ID NO:65.

36. The antibody or antigen binding fragment of any one of claims 21-35, wherein the antibody or antigen binding fragment binds to an ApoL1-containing complex, optionally, wherein the ApoL1-containing complex is Trypanosome Lytic Factor (TLF).

37. The antibody or antigen binding fragment of claim 36, wherein the TLF is endogenous human TLF.

38. The antibody or antigen binding fragment of claims 36 or 37, wherein the antibody or antigen binding fragment can bind to the ApoL1-containing complex under physiological conditions.

39. The antibody or antigen binding fragment of any one of claims 36-38, wherein the antibody or antigen binding fragment can bind to an ApoL1-containing complex in a subject, optionally wherein the subject is a human.

40. The antibody or antigen binding fragment of any one of claims 21-39, wherein the antibody is not a mouse IgG1 or IgG2a.

41. The antibody or antigen binding fragment of any one of claims 21-40 comprising one or more constant domains from an immunoglobulin constant region (Fc).

42. The antibody or antigen binding fragment of claim 41 wherein the constant domains are human constant domains.

43. The antibody or antigen binding fragment of claim 42 wherein the human constant domains are IgA, IgD, IgE, IgG or IgM domains.

44. The antibody or antigen binding fragment of claim 43 wherein human IgG constant domains are IgG1, IgG2, IgG3, or IgG4 domains.

45. The antibody or antigen binding fragment of any one of claims 21-44 wherein the antibody or antigen binding fragment is detectably labeled or comprises a conjugated toxin, drug, receptor, enzyme, receptor ligand.

46. The antibody or antigen binding fragment of any one of claim 21-45, wherein the antibody is a monoclonal antibody, a human antibody, a chimeric antibody, or a humanized antibody.

47. The antibody or antigen binding fragment of any one of claims 21-46, wherein the antibody is a bispecific, trispecific or multispecific antibody.

48. The antibody or antigen binding fragment of claim 47, wherein the bispecific, trispecific or multispecific antibody comprises a second antigen binding fragment that binds to a cell specific antigen.

49. A bispecific, trispecific or multispecific antibody comprising a first antigen binding fragment that binds to ApoL1 or an ApoL1-containing complex optionally a TLF and a second antigen binding fragment that binds to a cell specific antigen.

50. The antibody or antigen binding fragment of claims 48 or 49, wherein the cell specific antigen is a cancer or tumor antigen.

51. The antibody or antigen binding fragment of claim 50, wherein the cancer or tumor antigen is a blood cancer antigen or a solid tumor antigen optionally selected from Claudin 18.2, MUC1, Mesothelin (MSLN), Myoferlin (MYOF), and PMEL17.

52. The antibody or antigen binding fragment of claim 51, wherein the blood cancer antigen is selected from the group consisting of BCMA, CD38, CD319/SLAMF-7, TNFRSF17/BCMA, SYND1/CD138, CD229, CD47, CD123/IL3-RA, CD19, CD20, CD22, FcRH5, GPRC5D, CLL-1, PD-L1, and CTLA4.

53. The antibody or antigen binding fragment of any one of claims 48-52, wherein the cell specific antigen is BCMA.

54. The antibody or antigen binding fragment of claim 28, wherein the second antigen binding fragment comprises the three CDRs of the heavy chain variable domain of SEQ ID NO:41 or variant or humanized form thereof with at least 70, 80, 90, or 95% sequence identity thereto and the three CDRs of the light chain variable domain of SEQ ID NO:42 or variant or humanized form thereof with at least 70, 80, 90, or 95% sequence identity thereto.

55. The antibody or antigen binding fragment of claim 54, wherein the second antigen binding fragment comprises the six CDRs comprising the amino acid sequences:
CDR1H: SYAMS (SEQ ID NO:43) or variant or humanized form thereof with at least 70, 80, 90, or 95% sequence identity thereto,
CDR2H: AISGSGGSTYYADSVKG (SEQ ID NO:44) or variant or humanized form thereof with at least 70, 80, 90, or 95% sequence identity thereto,
CDR3H: VAPYFAPFDY (SEQ ID NO:45) or variant or humanized form thereof with at least 70, 80, 90, or 95% sequence identity thereto,
CDR1L: RASQSVSSSYLA (SEQ ID NO:46) or variant or humanized form thereof with at least 70, 80, 90, or 95% sequence identity thereto,
CDR2L: GASSRAT (SEQ ID NO:47) or variant or humanized form thereof with at least 70, 80, 90, or 95% sequence identity thereto, and
CDR3L: QQYGNPPLYT (SEQ ID NO:48) or variant or humanized form thereof with at least 70, 80, 90, or 95% sequence identity thereto.

56. The antibody or antigen binding fragment of any one of claims 53-55, wherein the second antigen binding fragment comprises a heavy chain variable domain comprising the amino acid sequence of SEQ ID NO:41 or variant or humanized form thereof with at least 70, 80, 90, or 95% sequence identity thereto and a light chain variable domain comprising the amino acid sequence of SEQ ID NO:42 or variant or humanized form thereof with at least 70, 80, 90, or 95% sequence identity thereto.

57. The antibody or antigen binding fragment of claim 56, wherein the second antigen binding fragment comprises a heavy chain variable domain comprising the amino acid sequence of SEQ ID NO:41 and a light chain variable domain comprising the amino acid sequence of SEQ ID NO:42,
optionally wherein the second antigen binding fragment comprises the amino acid sequence of SEQ ID NO:51.

58. The antibody or antigen binding fragment of any one of claims 49-56 comprising the amino acid sequence of SEQ ID NO:71 or variant or humanized form thereof with at least 70, 80, 90, or 95% sequence identity thereto and/or the amino acid sequence of SEQ ID NO:72 or variant or humanized form thereof with at least 70, 80, 90, or 95% sequence identity thereto.

59. The antibody or antigen binding fragment of any one of claims 49-56 comprising the amino acid sequence of SEQ ID NO:71 and SEQ ID NO:72.

60. The antibody or antigen binding fragment of any one of claims 49-56 comprising two copies each of the amino acid sequences of SEQ ID NO:70 and SEQ ID NO:72

61. An anti-ApoL1, anti-cell specific antigen IgG1-scFv bispecific chimeric antibody.

62. An anti-Hpr, anti-cell specific antigen IgG1-scFv bispecific chimeric antibody.

63. A nucleic acid encoding the antibody or antigen binding fragment of any one of claims 21-63.

64. The nucleic acid of claim 63 operably linked to an expression control sequence.

65. An expression vector comprising the nucleic acid of claims 63 or 64.

66. A cell comprising the nucleic acid of claims 63 or 64, or the expression vector of claim 65, optionally wherein the cell is a mammalian cell.

67. An immunocomplex comprising the antibody or antigen binding fragment of any one of claims 21-62 bound to ApoL1 or an ApoL1-containing complex, optionally wherein the complex is a TLF.

68. An immunocomplex comprising the antibody or antigen binding fragment of any one of claims 47-62.

69. A method of inducing cell death comprising contacting target cells with the immunocomplex of claims 67 or 68.

70. The method of claim 69, wherein the contacting occurs in vitro.

71. The method of claim 69, wherein the contracting occurs in vivo in a subject.

72. The method of claim 71, wherein the subject has cancer.

73. A pharmaceutical composition comprising the antibody or antigen binding fragment of any one of claims 21-62.

74. A method of treating cancer comprising administering the subject an effective amount of the antibody or antigen binding fragment of any one of claims 21-62.

75. The method of claim 74, comprising administering the subject an effective amount of the antibody or antigen binding fragment of any one of claims 22-62.

76. The method of claims 74 or 75, wherein the cancer is blood cancer.

77. The method of claim 76, wherein the cancer is multiple myeloma, leukemia (e.g., chronic lymphocytic leukemia, acute myeloid leukemia, acute lymphoblastic leukemia), non-Hodgkin lymphoma, Hodgkin lymphoma, myelodysplastic syndromes (MDS), myeloproliferative neoplasms (MPNs) (or a subcategory thereof, e.g., essential thrombocythemia (ET), myelofibrosis (MF) and polycythemia vera (PV), amyloidosis, Waldenstrom macroglobulinemia, or aplastic anemia.

78. The method of claims 74 or 75, wherein the cancer is a solid cancer.

79. A method of treating a subject in need thereof comprising administering the subject the composition of any one of claims 1-62.

80. The method of claim 79, wherein the subject has cancer.

81. The method of any one of claims 1-5 or 11-17 wherein the composition comprises the antibody of any one of claims 21-62.

82. The composition or method of any one of claims 1-81, wherein the target cells are mammalian cells.

83. The composition or method of claim 82, wherein the mammalian cells are infected cells.

84. The composition or method of claim 83, wherein the infected cells are infected with a virus, bacteria, or eukaryotic intracellular organism, optionally selected from HIV, Plasmodium falciparum, Toxoplasma gondii, Leishmania sp., Trypanosoma cruzi, Listeria monocytogenes, Chlamydia trachomatis, Coxiella burnetti, Mycobacterium tuberculosis, and Trichomonas vaginalis.

85. The composition or method of any one of claims 1-81, wherein the target cells are non-mammalian cells.

86. The composition or method of claim 85, wherein the non-mammalian cells are bacteria, fungi, or non-mammalian eukaryotic cells.

87. The composition or method of claim 86, wherein the non-mammalian cells are not Trypanosoma.

88. The composition or method of any one of the foregoing claims wherein the subject is a mammal, optionally a human.
